(19) United States
(12) Patent Application Publication
(10) Pub. No.: US 2021/0145468 A1
(43) Pub. Date: May 20, 2021

(54) SUSPENSION GLOSSOPEXY, GLOSSOMANDIBULOPEXY, GLOSSOHYOIDOPEXY, AND HYOIDOMANDIBULOPEXY RELATED METHODS, DEVICES, AND APPARATUSES

(71) Applicant: Erik S. KASS, Bethesda, MD (US)

(72) Inventor: Erik S. KASS, Bethesda, MD (US)

(21) Appl. No.: 17/156,589

(22) Filed: Jan. 24, 2021

Related U.S. Application Data
(63) Continuation-in-part of application No. 15/857,832, filed on Dec. 29, 2017.
Application No. 15/857,832 is a continuation-in-part of application No. 15/723,317, filed on Oct. 3, 2017.
(60) Provisional application No. 62/975,989, filed on Feb. 13, 2020.
(60) Provisional application No. 62/965,178, filed on Jan. 24, 2020.
(60) Provisional application No. 62/403,848, filed on Oct. 4, 2016.

(51) Int. Cl. A61B 17/24; A61F 5/56

(52) U.S. Cl. CPC A61B 17/24; A61B 2017/00814; A61F 5/566

(57) ABSTRACT

The disclosed embodiments include apparatuses, devices, and methods for treating a breathing disorder. The method comprises creating an anchor hole at a location within or subjacent to a mandible bone, and positioning an elastic elongate member through the anchor hole, the elongate member having first and second ends at an entrance of the anchor hole and a loop in a region of a pharynx. The method further comprises connecting a retractor member at or near an end of the loop of the elastic elongate member in a region of the tongue, and connecting an anchor member at or near the ends of the elastic elongate member at the entrance of the anchor hole. In the method, at least one of the elastic elongate member, the retractor member, and the anchor member interact to distribute a force on the tongue and the force prevents obstruction of an airway.

### CROSS-REFERENCE TO RELATED APPLICATION

This application is a continuation-in-part of U.S. patent application Ser. No. 15/857,832, filed Dec. 29, 2017, in the United States Patent and Trademark Office, which is a continuation-in-part of U.S. patent application Ser. No. 15/723,317, filed Oct. 3, 2017, in the United States Patent and Trademark Office, which claims the benefit of priority under 35 U.S.C. § 119 to U.S. Provisional Application No. 62/403,848, filed Oct. 4, 2016, in the United States Patent and Trademark Office, the entire contents of all of which are hereby incorporated by reference. In addition, this application claims the benefit of priority under 35 U.S.C. § 119 to U.S. Provisional Application No. 62/965,178, filed Jan. 24, 2020, and U.S. Provisional Application No. 62/975,989, filed Feb. 13, 2020, in the United States Patent and Trademark Office, the entire contents of both of which are hereby incorporated by reference.

### FIELD

This present disclosure relates to suspension glossomandibulopexy, glossopexy, glossohyoidoplexy, and hyoidomandibulopexy. Particularly, the present disclosure relates to suspension glossomandibulopexy, suspension glossopexy, suspension glossohyoidoplexy, and suspension hyoidomandibulopexy to combat snoring and/or mitigate obstructive sleep apnea.

### BACKGROUND

Generally, obstructive sleep apnea is a breathing disorder characterized by snoring and apnea caused by upper airway collapse and obstruction during sleep. During normal sleep, the muscles of the upper part of the throat keep the airway open to permit air flow into the lungs. With obstructive sleep apnea, the muscles of the soft palate, the base of tongue and the uvula, can relax during sleep. In some cases, the relaxed tissues may vibrate as air flows past the tissues during breathing, resulting in snoring. In more serious cases, the airway can become blocked, making breathing labored and noisy, or even causing it to stop altogether. These breathing pauses are almost always accompanied by snoring between apnea episodes.

Obstructive sleep apnea can result in diminished health, in part, because the lack of air intake into the lungs results in lower levels of oxygen and increased levels of carbon dioxide in the blood. The altered levels of oxygen and carbon dioxide alert the brain to resume breathing and cause arousal. The frequent interruptions of deep, restorative sleep often lead to early morning headaches, excessive daytime sleepiness, depression, irritability, and difficulty with learning and memory. For those with moderate or severe obstructive sleep apnea, there is an increased incidence of diabetes, heart attacks, hypertension and strokes.

The disclosed embodiments provide for simple, cost-effective, minimally invasive devices and methods to reduce or prevent snoring and obstructive sleep apnea with a focus on the soft palate, tonsil, and tongue base.

### SUMMARY

In some example embodiments, the present disclosure is directed to a method for treating a condition of an airway of a patient, comprising: positioning a first elastic elongate member in an oral cavity of the patient, the first elastic elongate member having a first end and a second end in the oral cavity and a loop in a region of a pharynx of the patient; connecting a first retractor member at or near the loop of the first elastic elongate member; and connecting a first anchor member at or near the first and second ends of the first elastic elongate member in the oral cavity, wherein at least one of the first elastic elongate member, the first retractor member, and the first anchor member interact to distribute a force on a tongue base and the force prevents obstruction of an airway of the patient.

In further exemplary embodiments, the disclosure is directed to a method for treating a condition of an airway of a patient, comprising: positioning a first elastic elongate member in an oral cavity of the patient, the first elastic elongate member having a first end in the oral cavity and a second end in a region of a pharynx of the patient; connecting a first anchor member at or near the first end of the first elastic elongate member; and connecting a first retractor member at or near the second end of the elastic elongate member in a region of a tongue of the patient, wherein at least one of the first elastic elongate member, the first retractor member, and the first anchor member interact to distribute a force on a tongue base and the force prevents obstruction of an airway of the patient.

In further exemplary embodiments, the disclosure is directed to a method for treating a condition of an airway of a patient, comprising: positioning an elastic elongate member through soft tissue of a tongue of the patient, the elastic elongate member having a first end and a second end in an oral cavity of the patient a loop in a region of a pharynx of the patient; and connecting a first anchor member at or near the first end of the elastic elongate member, wherein at least one of the elastic elongate member and the first anchor member interact to distribute a force on a tongue base and the force prevents obstruction of an airway of the patient.

In further exemplary embodiments, the disclosure is directed to a method for treating a condition of an airway of a patient, comprising: creating a first anchor hole at a first predetermined location within or subjacent to a mandible bone of the patient; positioning a first elastic elongate member through the first anchor hole, the first elongate member having first and second ends at an entrance of the first anchor hole and a loop in a region of a pharynx of the patient; connecting a first retractor member at or near an end of the loop of the first elastic elongate member in a region of a tongue of the patient; and connecting a first anchor member at or near the first and second ends of the first elastic elongate member at the entrance of the first anchor hole, wherein at least one of the first elastic elongate member, the first retractor member, and the first anchor member interact to distribute a force on the tongue and the force prevents obstruction of an airway of the patient.

In further exemplary embodiments, the disclosure is directed to a method for treating a condition of an airway of a patient, comprising: creating a first anchor hole at a first predetermined location within or subjacent to a mandible bone of the patient; positioning a first elastic elongate member through the first anchor hole, the first elongate member having a first end at an entrance of the first anchor hole and a second end in a region of a pharynx of the patient; connecting a first anchor member at or near the first end of the first elastic elongate member at the entrance of the first anchor hole; and connecting a first retractor member at or near the second end of the first elastic elongate member in a region of a tongue of the patient, wherein at least one of the first elastic elongate member, the first retractor member, and the first anchor member interact to distribute a force on the tongue and the force prevents obstruction of the an airway of the patient.

In further exemplary embodiments, the disclosure is directed to a method for treating a condition of an airway of a patient, comprising: creating a first anchor hole at a first predetermined location within or subjacent to a mandible bone of the patient; positioning a first elastic elongate member through the first anchor hole, the first elongate member having a first end at an entrance of the first anchor hole and a second end in a region of a pharynx of the patient; connecting a first anchor member at or near the first end of the first elastic elongate member at the entrance of the first anchor hole; and connecting a first retractor member at or near the second end of the first elastic elongate member in a region of a tongue of the patient, wherein at least one of the first elastic elongate member, the first retractor member, and the first anchor member interact to distribute a force on the tongue and the force prevents obstruction of an airway of the patient.

In further exemplary embodiments, the disclosure is directed to a method for treating a condition of an airway of a patient, comprising: creating a first anchor hole at a first predetermined location within or subjacent to a mandible bone of the patient; creating a second anchor hole at a second predetermined location within or subjacent to the mandible bone of the patient; positioning an elastic elongate member through the first and second anchor holes, the first elongate member having a first end at an entrance of the first anchor hole, a second end at an entrance of the second anchor hole, and a loop in a region of a pharynx of the patient; and connecting a first anchor member at or near the first end the elastic elongate member, wherein at least one of the elastic elongate member and the first anchor member interact to distribute a force on a tongue of the patient and the force prevents obstruction of an airway of the patient.

In further exemplary embodiments, the disclosure is directed to a method for treating a condition of an airway of a patient, comprising: positioning a first elastic elongate member in a soft tissue of a tongue of the patient, the first elastic elongate member having first and second ends in a midline region of a hyoid bone of the patient and a loop in a region of a pharynx of the patient; and connecting an anchor member at or near the first and second ends of the first elastic elongate member in the midline region of the hyoid bone, wherein at least one of the first elastic elongate member and the anchor member interact to distribute a force on the tongue of the patient and the force prevents obstruction of the airway of the patient.

In further exemplary embodiments, the disclosure is directed to a method for treating a condition of an airway of a patient, comprising: positioning a first elastic elongate member in a soft tissue of a tongue of the patient, the first elastic elongate member having a first end in a midline region of a hyoid bone of the patient and a second end in a region of a pharynx of the patient; connecting an anchor member at or near the first end of the first elastic elongate member in the midline region of the hyoid bone; and connecting a first retractor member at or near the second end of the first elastic elongate member in a region of a tongue of the patient, wherein at least one of the first elastic elongate member and the anchor member interact to distribute a force on the tongue of the patient and the force prevents obstruction of the airway of the patient.

In further exemplary embodiments, the disclosure is directed to a method for treating a condition of an airway of a patient, comprising: creating a first anchor hole at a first predetermined location of a mandible bone of the patient; positioning a first elastic elongate member through the first anchor hole, the first elongate member having first and second ends at an entrance of the first anchor hole and a loop in a midline region of a hyoid bone of the patient; and connecting a first anchor member at or near the first and second ends of the first elastic elongate member at the entrance of the first anchor hole, wherein at least one of the first elastic elongate member and the first anchor member interact to distribute a force on the hyoid bone and the force prevents obstruction of an airway of the patient.

### BRIEF DESCRIPTION OF THE DRAWINGS

The above and other objects and features will become apparent from the following description with reference to the following figures, wherein like reference numerals refer to like parts throughout the various figures unless otherwise specified. The drawings are not necessarily to scale, emphasis instead being placed upon illustrating the principles of the disclosed embodiments. In the drawings:

FIG. 1A is a cross-sectional side (saggital) view of a human head during nasal breathing;

FIG. 1B is a cross-sectional side view of a human head depicting obstruction of the airway;

FIG. 1C is a front view of certain features of the upper respiratory system;

FIGS. 2A-2I are front views of a human mouth illustrating a multi-component device for use in suspension uvulopalatopexy, according to example embodiments;

FIGS. 3A-3D are front views of a human mouth illustrating a multi-component device for use in suspension glossopexy, according to example embodiments;

FIGS. 3E-H are front views of a human mouth illustrating a multi-component device for use in suspension uvulopalatopexy, according to example embodiments;

FIGS. 4A-4C are schematics of an oral stud for use in suspension uvulopalatopexy, according to example embodiments;

FIGS. 5A-5B are schematics of an oral stud with an insertion blade, according to example embodiments;

FIGS. 6A-6N are schematics of alternative stud heads of an oral stud, according to example embodiments;

FIG. 7A-7D are diagrams illustrating connectors for connecting oral studs, according to example embodiments;

FIG. 8 is a block diagram of an oral stud placement gun, according to example embodiments;

FIG. 9A is a schematic illustrating the barrel of an oral stud placement gun loaded with an oral stud, according to example embodiments;

FIG. 9B is a schematic illustrating a blown up view of cross-section A-A of FIG. 9A and a stud drive shaft and a blade drive shaft, according to example embodiments;

FIG. 9C is a schematic illustrating a blown up view of cross-section B-B of FIG. 9A and a stud drive shaft and a blade drive shaft, according to example embodiments;

FIG. 9D is a schematic illustrating a blown up view of cross-section C-C of FIG. 9A and a stud drive shaft and a blade drive shaft, according to example embodiments;

FIG. 9E is a schematic illustrating a blown up view of cross-section D-D of FIG. 9A and a stud drive shaft and a blade drive shaft, according to example embodiments;

FIG. 9F is a schematic illustrating a blown up view of cross-section E-E of FIG. 9A and a stud drive shaft and a blade drive shaft, according to example embodiments;

FIG. 9G is a schematic illustrating a blown up view of cross-section F-F of FIG. 9A and a stud drive shaft and a blade drive shaft, according to example embodiments;

FIG. 9H is a schematic illustrating a blown up view of cross-section G-G of FIG. 9A and a stud drive shaft and a blade drive shaft, according to example embodiments;

FIG. 9I is a schematic illustrating a blown up view of cross-section H-H of FIG. 9A and a stud drive shaft and a blade drive shaft, according to example embodiments;

FIG. 10 is a flowchart of a method of suspension uvulopalatopexy using a multi-component device, according to example embodiments;

FIG. 11 illustrates an oral stud loaded in an oral stud placement gun when it is placed in contact with an anchor or support site, according to example embodiments;

FIG. 12 illustrates an oral stud loaded in an oral stud placement gun when it is engaged with an anchor or support site, according to example embodiments;

FIG. 13 illustrates an oral stud loaded in an oral stud placement gun when it advances through an anchor or support site, according to example embodiments;

FIG. 14 illustrates an oral stud loaded in an oral stud placement gun when it is deployed into an anchor or support site, according to example embodiments;

FIGS. 15A-15C are side views illustrating suspension glossopexy using a multi-component device, according to example embodiments;

FIG. 16A-16F are front views of a human mouth illustrating placement of certain components of a multi-component device, according to example embodiments;

FIG. 17A-17C are front views of a human mouth illustrating placement of certain components of a multi-component device, according to example embodiments;

FIGS. 18A-18B, 19A-19B, 20A-20B, 21A-21B, 22A-22B, 23A-23B, 24A-24B, 25A-25B, 26A-26B, and 27A-27B are front and side views illustrating suspension glossopexy using a multi-component device, according to example embodiments;

FIGS. 28A-28B are side views of a human mouth illustrating a method of placement of a multi-component device for suspension glossopexy, according to example embodiments;

FIGS. 29A-29B are views of the insertion tool used in connection with the placement of the components of the multi-component device, consistent with example embodiments;

FIG. 30 illustrates an endoscopic helmet camera system for use with the placement of the components of the multi-component device, consistent with example embodiments;

FIGS. 31A-31B, 32A-32B, 33A-33B, 34A-34B, 35A-35B, 36A-36B, 37A-37B, and 38A-38B are front and side views illustrating suspension glossomandibulopexy using a multi-component device, according to example embodiments;

FIGS. 39A-39C are side views of a human mouth illustrating a method of placement of a multi-component device for suspension glossomandibulopexy, according to example embodiments;

FIGS. 40A-40B are front and side views illustrating suspension glossohyoidopexy using a multi-component device, according to example embodiments;

FIG. 41 includes front and side views illustrating suspension glossohyoidopexy using a multi-component device, according to example embodiments

FIGS. 42A-42B are front and side views illustrating suspension glossohyoidopexy using a multi-component device, according to example embodiments;

FIGS. 43A-43C are side views of a human mouth illustrating a method of placement of a multi-component device suspension glossohyoidopexy, according to example embodiments; and

FIGS. 44-45 are front and side views illustrating suspension hyoidomandibulopexy using a multi-component device, according to example embodiments.

### DETAILED DESCRIPTION

Various exemplary embodiments will be described in detail with reference to the accompanying drawings. The inventive concept, however, may be embodied in various different forms, and should not be construed as being limited only to the illustrated embodiments. Accordingly, known processes, elements, and techniques are not described with respect to some of the embodiments of the disclosure. Unless otherwise noted, like reference numerals denote like elements throughout the attached drawings and written description, and thus descriptions will not be repeated. In the drawings, the sizes and relative sizes of layers and regions may be exaggerated for clarity.

The terminology used herein is for the purpose of describing particular embodiments only and is not intended to be limiting of the disclosure. As used herein, the singular forms “a,” “an,” and “the” are intended to include the plural forms as well, unless the context clearly indicates otherwise. It will be further understood that the terms “comprises” and/or “comprising” or “includes” and/or “including,” when used in this specification, specify the presence of stated features, regions, integers, steps, operations, elements, components, and/or groups, but do not preclude the presence or addition of one or more other features, regions, integers, steps, operations, elements, components, and/or groups thereof In addition, unless the context indicates otherwise, steps described in a particular order need not occur in that order. As used herein, the term “and/or” includes any and all combinations of one or more of the associated listed items.

It will be understood that, although the terms “first,” “second,” “third,” etc., may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the disclosure.

As will be understood, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood, all language such as “up to,” “at least,” “greater than,” “less than,” and the like include the number recited and refer to ranges which can be subsequently broken down into subranges as discussed above. Finally, as will be understood, a range includes each individual member. Thus, for example, a group having 1-3 members refers to groups having 1, 2, or 3 members. Similarly, a group having 1-5 members refers to groups having 1, 2, 3, 4, or 5 members, and so forth.

Unless otherwise defined, all terms (including technical and scientific terms) used herein have the same meaning as commonly understood by one of ordinary skill in the art to which this disclosure belongs. It will be further understood that terms, such as those defined in commonly used dictionaries, should be interpreted as having a meaning that is consistent with their meaning in the context of the relevant art and/or the present specification and will not be interpreted in an idealized or overly formal sense unless expressly so defined herein.

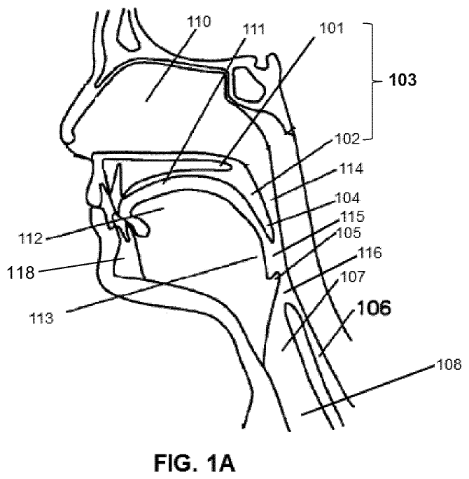

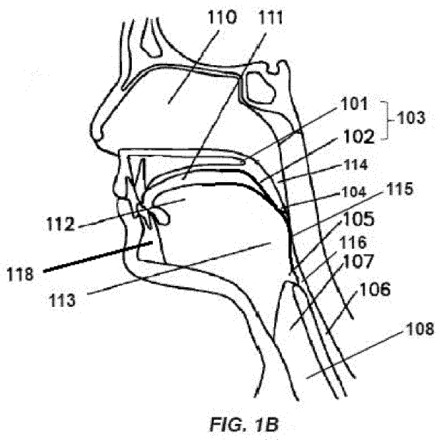

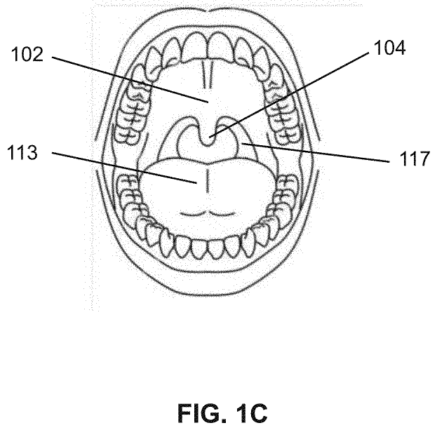

FIGS. 1A and 1B are cross-sectional side views of a human head, illustrating the upper respiratory system. FIG. 1C is a front view of certain features of the upper respiratory system. In particular, FIGS. 1A-1C illustrate the structures that perform the functions of breathing and swallowing, including the hard palate 101 and the soft palate 102 (collectively referred to as the palate 103), the uvula 104 (which is contiguous with the lower portion of the soft palate 102), the epiglottis 105, the esophagus 106, the larynx 107, the trachea 108, the nasal cavity 110, the oral cavity (mouth) 111, the tongue 112, the tongue base 113, the pharynx, which is comprised of the nasopharynx 114, the oropharynx 115, the hypopharynx 116, and the lateral pharyngeal walls 117, and lastly, mandible 118.

As shown in FIGS. 1A and 1B, the palate 103 is located in the upper portion of the oral cavity 111, and it separates the oral cavity 111 from the nasal cavity 110. The anterior two-thirds of the palate 103 is the bony hard palate 101, and the posterior one-third of the palate 103 is known as the soft palate 102. The soft palate 102, which is comprised of muscle and aponeurosis, is suspended from the posterior border of the hard palate 101 and extends posteroinferiorly. The uvula 104 hangs from the posterior region of the soft palate 102.

The nasopharynx 114, which is located posterosuperior to the soft palate 102, lies posterior to the nasal cavity 110, extending from the base of the skull to the soft palate 102. The oropharynx 115 extends from the hard palate 101 to the hyoid bone (not illustrated). The oropharynx 115 communicates with the nasopharynx 114 superiorly, the oral cavity 111 anteriorly, and the hypopharynx 116 inferiorly.

The tongue 112 is located in the lower portion of the oral cavity 111. The posterior portion of the tongue 112 forms the tongue base 113. The epiglottis 105 is a thin structure immediately posterior to the tongue base 113. Although not illustrated, when an individual swallows, the epiglottis 105 covers the entrance of the larynx 107, thereby preventing food or liquids from entering the airway.

As shown in FIG. 1C, the lateral pharyngeal walls 117 (including the palatoglossal and palatopharyngeal arches) form the lateral walls of the oropharynx 115. The palatoglossal arch is a fold of mucosa that runs from the soft palate 102 to the tongue 112. The palatopharyngeal arch is a fold of mucosa posterior to the palatoglossal arch that attaches from the soft palate 102 to the pharyngeal wall. The hypopharynx 116 lies posterior to the larynx 107, extending from the upper border of the epiglottis 105 to the lower border of the cricoid cartilage (not illustrated), and serves as the entrance to the esophagus 106.

FIG. 1A illustrates normal breathing during which the upper airway remains open, allowing air to flow unobstructed. During normal breathing the soft palate 102 naturally falls, the epiglottis 105 opens, and air may enter the trachea 108 via the nasal cavity 110 (or oral cavity 111, during mouth breathing).

FIG. 1B illustrates occurrence of obstructive sleep apnea (OSA) in a patient. When OSA occurs, the soft tissue of the upper airway collapses, and the upper airway is obstructed, resulting in insufficient airflow and even apnea. As illustrated in FIG. 1B, the soft palate 102, the uvula 104, and/or the lateral pharyngeal walls may collapse backwards, causing the passage between soft palate 102 and oropharynx 115 to become narrow or blocked. At the same time, soft tissues of the tongue base 113 may collapse, and the passage between tongue base 113 and soft palate 102 in the oral cavity 111 may become narrow or blocked, resulting in insufficient airflow during breathing and even OSA. In some cases, the collapse of the tongue base 113 not only directly causes the passage between the tongue base 113 and the soft palate 102 to become narrow or blocked, but also causes the passage between the soft palate 102 and the oropharynx 115 to narrow or become blocked.

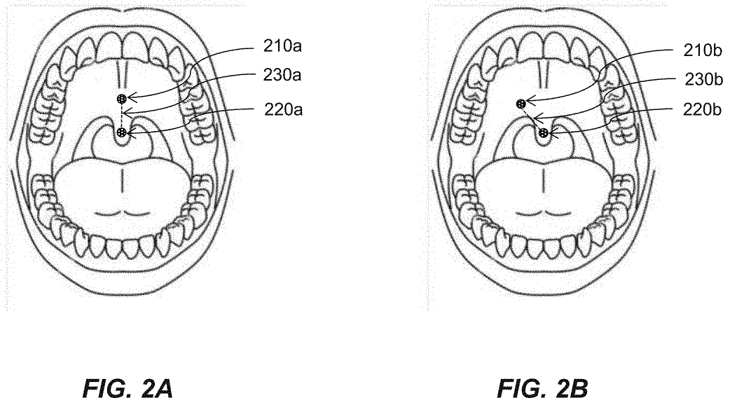

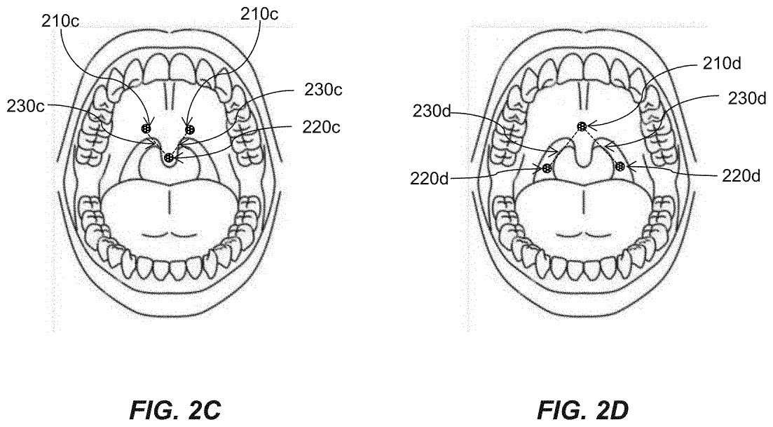

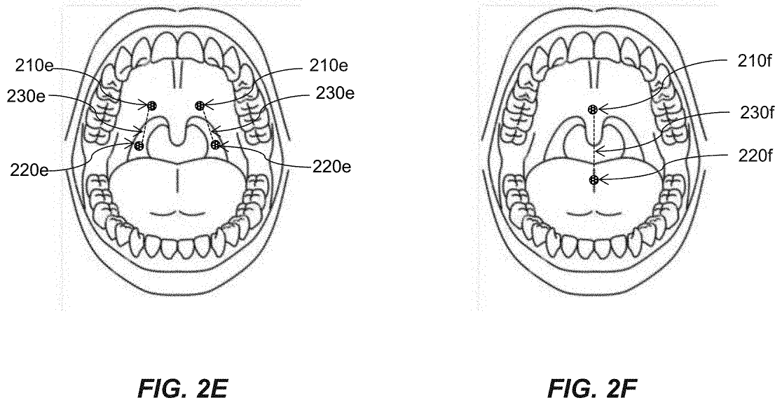

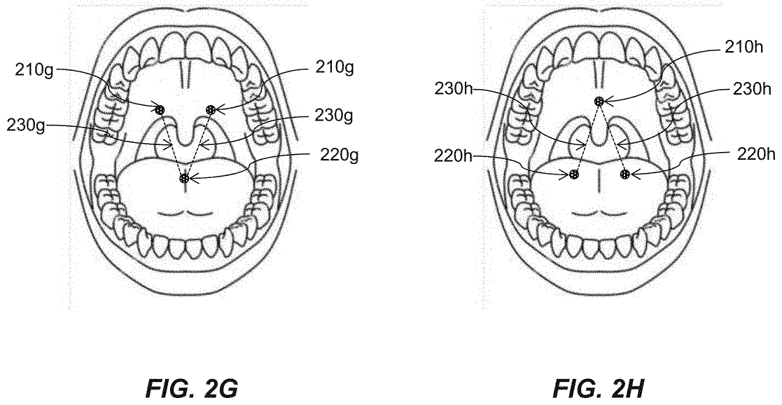

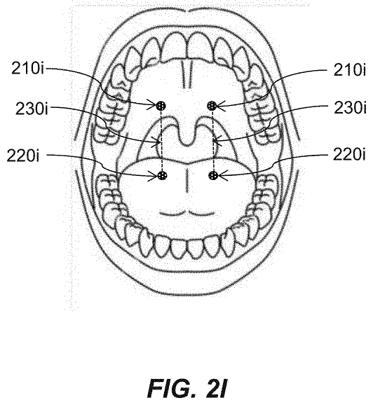

FIGS. 2A-2I are diagrams illustrating placement of components of a multi-component device used in suspension uvulopalatopexy, consistent with certain exemplary embodiments. Specifically, each of FIGS. 2A through 2I illustrate a multi-component device used to dynamically support and/or retract the soft palate 102, the tongue 112, and/or the lateral pharyngeal walls 117. Generally, the multi-component device includes one or more oral studs inserted into a structure that provides anchoring (e.g., anchor studs 210), one or more oral studs inserted into a structure that are to be supported (e.g., support studs 220), and one or more external elastic connectors 230 that mechanically couple one or more anchor studs 210 to one or more support studs 220. For ease of description, structures that provide anchoring may be referred to herein as anchor or target structures, and structures that are supported by the anchor structures may be referred to herein as support structures. In the disclosed embodiments, support structures may include the uvula 104, soft palate 102, the tongue 112, and/or the lateral pharyngeal walls 117, and anchor structures may include the soft palate 102, mandible (lower teeth) 118, and/or hard palate (upper teeth) 101.

As illustrated in FIGS. 2A-2I, the oral studs (e.g., anchor studs 210 and support studs 220) and elastic connectors 230 may work together to affect a position of the soft palate 102, the tongue 112, and/or the lateral pharyngeal walls 117. For example, the anchor studs 210, support studs 220, and elastic connectors 230 may maintain a position of, or bring forward, the soft palate 102, the tongue 112, and/or the lateral pharyngeal walls 117, thereby maintaining an open passage through the oropharynx 115. At rest, the arrangement of the anchor studs 210 and support studs 220 and the pulling forces applied by each connector 230 may support/displace one or more of the soft palate 102, the tongue 112, and/or the lateral pharyngeal walls 117 in an aerodynamically favorable manner to enhance breathing during sleep while at the same time accommodating the natural movements of these muscular structures during speech, breathing, and swallowing by stretching/contracting passively according to the degree of tension exerted by the contractions of the local musculature.

FIG. 2A illustrates an embodiment including two oral studs, i.e., one anchor stud 210a and one support stud 220a. As shown in FIG. 2A, the anchor stud 210a may be inserted through the soft palate 102 at a midline of the soft palate 102, and the support stud 220a may be inserted through another region of the soft palate 102 or the uvula 104. In the embodiment of FIG. 2A, the anchor stud 210a may be inserted at a midline of the soft palate 102, posteriorly to and near the hard palate 101. The anchor stud 210a and the support stud 220a may be connected to one another with a connector 230a external to the palate 103. The connector 230a may extend across the external surface of the palate 103. The connector 230a may be used to alter the position of the uvula 104 and, in particular, move the uvula 104 anteriorly away from the pharynx. In the embodiment illustrated by FIG. 2A, due to the positioning of the anchor stud 210a and the support stud 220a, the uvula 104 may be shifted slightly forward, while maintaining a centered position in the oral cavity 111.

FIG. 2B illustrates an embodiment including two oral studs, i.e., one anchor stud 210b and one support stud 220b. As shown in FIG. 2B, the anchor stud 210b may be inserted through the soft palate 102, and the support stud 220b may be inserted through another region of the soft palate 102 or the uvula 104. In the embodiment of FIG. 2B, the anchor stud 210b may be inserted at a position offset from a midline of the soft palate 102, posteriorly to and near the hard palate 101. The anchor stud 210b and the support stud 220b may be connected to one another with a connector 230b external to the palate 103. The connector 230b may be used to alter the position of the uvula 104 and, in particular, move the uvula 104 anteriorly away from the pharynx. In the embodiment illustrated by FIG. 2B, due to the positioning of the anchor stud 210b and the support stud 220b, the uvula 104 may be shifted forward and slightly off-center in the oral cavity 111.

FIG. 2C illustrates an embodiment including three oral studs, i.e., two anchor studs 210c and one support stud 220c. As shown in FIG. 2C, the support stud 220c may be inserted through the uvula 104 at a midline of the uvula 104, and the two anchor studs 210c may be inserted through other regions of the soft palate 102. In the embodiment of FIG. 2C, the anchor studs 210c may be inserted at positions offset from a midline of the soft palate 102, posteriorly to and near the hard palate 101. In some embodiments, the anchor studs 210c may be inserted equidistant from and on opposite sides of the midline of the soft palate 102. The anchor studs 210c and the support stud 220c may be connected with connectors 230c external to the palate 103. For example, a first connector 230c may connect the support stud 220c to a first one of the anchor studs 210c, and a second connector 230c may connect the support stud 220c to a second one of the anchor studs 210c. The connector 230c may be used to alter the position of the uvula 104 and, in particular, move the uvula 104 and/or soft palate 102 anteriorly away from the pharynx. In the embodiment illustrated by FIG. 2C, the uvula 104 may be shifted slightly forward, while maintaining a centered position in the oral cavity 111.

FIG. 2D illustrates an embodiment including three oral studs, i.e., two support studs 220d and one anchor stud 210d. As shown in FIG. 2D, the support studs 220d may be inserted through the lateral pharyngeal walls 117, with one on either side of the soft palate 102, and the one anchor stud 210d may be inserted through an upper region of the soft palate 102. In the embodiment of FIG. 2D, the anchor stud 210d may be inserted at a midline of the soft palate 102, posteriorly to and near the hard palate 101. The anchor stud 210d and the support studs 220d may be connected to one another with connectors 230d external to the palate 103. For example, a first connector 230d may connect the anchor stud 210d to a first one of the support studs 220d, and a second connector 230d may connect the anchor stud 210d to a second one of the support studs 220d. The connectors 230d may be used to alter the position of the lateral pharyngeal walls 117 and, in particular, move the lateral pharyngeal walls 117 anteriorly away from the pharynx. In the embodiment illustrated by FIG. 2D, the lateral pharyngeal walls 117 may be shifted slightly forward in the oral cavity 111.

FIG. 2E illustrates an embodiment including four oral studs, i.e., two anchor studs 210e and two support studs 220e. As shown in FIG. 2E, the support studs 220e may be inserted through the lateral pharyngeal walls 117, with one on either side of the soft palate 102, and the two anchor studs 210e may be inserted through an upper region of the soft palate 102. In the embodiment of FIG. 2E, the anchor studs 210e may be inserted at positions offset from a midline of the soft palate 102, posteriorly to and near the hard palate 101. In some embodiments, the anchor studs 210e may be inserted equidistant from and on opposite sides of the midline of the soft palate 102. The anchor studs 210e and the support studs 220e may be connected to one another with connectors 230e external to the palate 103. For example, a first connector 230e may connect a first one of the support studs 220e to a first one of the anchor studs 210e, and a second connector 230e may connect a second one of the support studs 220e to a second one of the anchor studs 210e. The connectors 230e may be used to alter the position of the lateral pharyngeal walls 117 and, in particular, move the lateral pharyngeal walls 117 anteriorly away from the pharynx. In the embodiment illustrated by FIG. 2E, the lateral pharyngeal walls 117 may be shifted slightly forward in the oral cavity 111.

FIG. 2F illustrates an embodiment including two oral studs, i.e., one anchor stud 210f and one support stud 220f. As shown in FIG. 2F, the support stud 220f may be inserted through the tongue 112 or tongue base 113, and the anchor stud 210f may be inserted through an upper region of the soft palate 102. The support stud 220f may be inserted at a midline position of the tongue 112 or tongue base 113. In the embodiment of FIG. 2F, the anchor stud 210f may be inserted at a midline position of the soft palate 102, posteriorly to and near the hard palate 101. The anchor stud 210f and the support stud 220f may be connected to one another with connector 230f external to the palate 103. The connector 230f may be used to alter the position of the tongue 112 and, in particular, move the tongue 112 anteriorly away from the pharynx. In the embodiment illustrated by FIG. 2F, the tongue 112 may be shifted slightly forward in the oral cavity 111.

FIG. 2G illustrates an embodiment including three oral studs, i.e., one support stud 220g and two anchor studs 210g. As shown in FIG. 2G, the support stud 220g may be inserted through the tongue 112 or tongue base 113, and the two anchor studs 210g may be inserted through an upper region of the soft palate 102. The support stud 220g may be inserted at a midline position of the tongue 112 or tongue base 113. In the embodiment of FIG. 2G, the anchor studs 210g may be inserted at positions offset from a midline of the soft palate 102, posteriorly to and near the hard palate 101. In some embodiments, the anchor studs 210g may be inserted equidistant from and on opposite sides of the midline of the soft palate 102. The support studs 220g and the anchor studs 210g may be connected to one another with connectors 230g external to the palate 103. For example, a first connector 230g may connect the support stud 220g to a first one of the anchor studs 210g, and a second connector 230g may connect the support stud 220g to a second one of the anchor studs 210g. The connectors 230g may be used to alter the position of the tongue 112 and, in particular, move the tongue 112 anteriorly away from the pharynx. In the embodiment illustrated by FIG. 2G, the tongue 112 may be shifted slightly forward in the oral cavity 111.

FIG. 2H illustrates an embodiment including three oral studs, i.e., two support studs 220h and one anchor stud 210h. As shown in FIG. 2H, the support studs 220h may be inserted through the tongue 112 or tongue base 113, with one on either side of a midline of the tongue 112, and the anchor stud 210h may be inserted through an upper region of the soft palate 102. In the embodiment of FIG. 2H, the anchor stud 210h may be inserted at a midline of the soft palate 102, posteriorly to and near the hard palate 101. The support studs 220h and the anchor stud 210h may be connected to one another with connectors 230h external to the palate 103. For example, a first connector 230h may connect a first one of the support studs 220h to the anchor stud 210h, and a second connector 230h may connect a second one of the support studs 220h to the anchor stud 210h. The connectors 230h may be used to alter the position of the tongue 112 and, in particular, move the tongue 112 anteriorly away from the pharynx. In the embodiment illustrated by FIG. 2H, the tongue 112 may be shifted slightly forward in the oral cavity 111.

FIG. 2I illustrates an embodiment including four oral studs, i.e., two anchor studs 210i and two support studs 220i. As shown in FIG. 2I, the support studs 220i may be inserted through the tongue 112 or tongue base 113, with one on either side of a midline of the tongue 112, and the two anchor studs 210i may be inserted through an upper region of the soft palate 102. In the embodiment of FIG. 2I, the anchor studs 210i may be inserted at positions offset from a midline of the soft palate 102, posteriorly to and near the hard palate 101. In some embodiments, the anchor studs 210i may be inserted equidistant from and on opposite sides of the midline of the soft palate 102. The anchor studs 210i and the support studs 220i may be connected to one another with connectors 230i external to the palate 103. For example, a first connector 230i may connect a first one of the support studs 220i to a first one of the anchor studs 210i, and a second connector 230i may connect a second one of the support studs 220i to a second one of the anchor studs 210i. The connectors 230i may be used to alter the position of the tongue 112 and, in particular, move the tongue 112 anteriorly away from the pharynx. In the embodiment illustrated by FIG. 2I, the tongue 112 may be shifted slightly forward in the oral cavity 111.

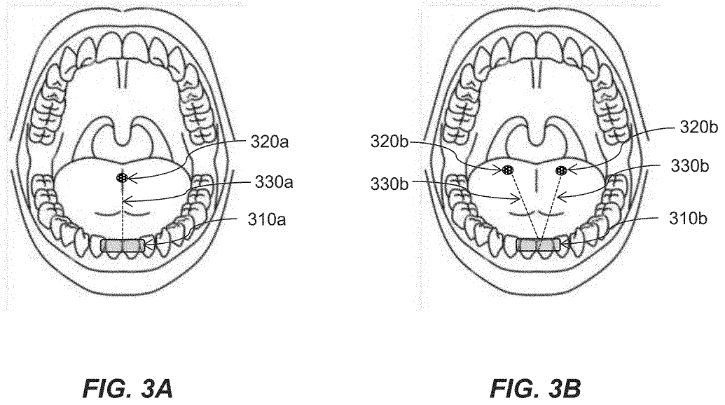

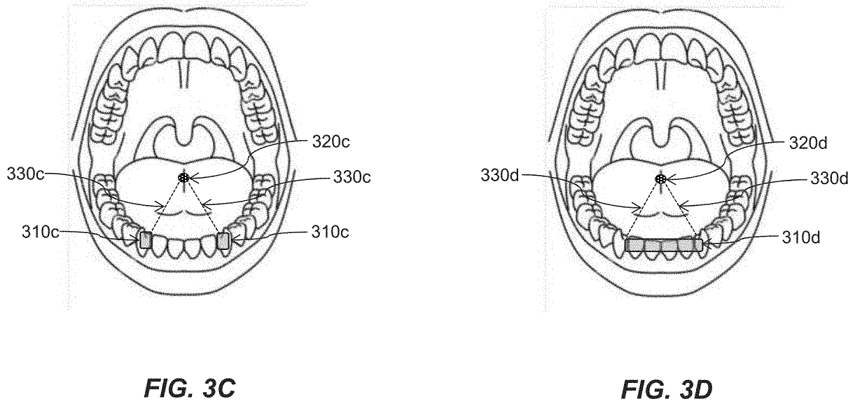

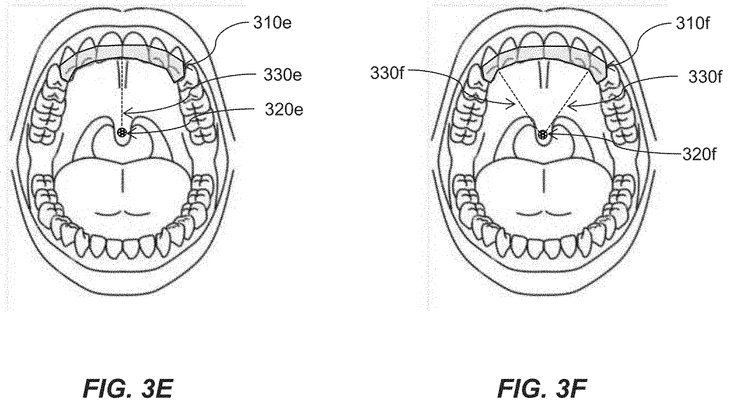

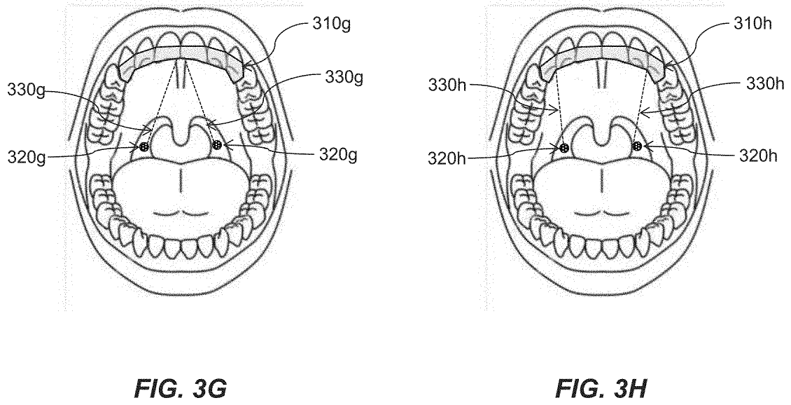

FIGS. 3A-3D are front views of a human head to illustrate placement of components of a multi-component device used in suspension glossopexy, consistent with certain exemplary embodiments. In the embodiments illustrated by FIGS. 3A-3D, the multi-component device includes at least one oral stud 320 inserted into a tongue 112 or tongue base 113, a securement to the mandible 118 by attachment or insertion to a mandible structure that provides support (e.g., teeth or bones), and one or more external elastic connectors 330 that mechanically couple at least one oral stud 320 to the mandible securement (e.g., dental securement 310). In the embodiments of FIGS. 3A-3D, the dental securement 310 may be removably attached (e.g., placed over the patient's teeth) or permanently attached (e.g., bonded or glued to the teeth, embedded in bone, etc.), such that the patient's teeth or jaw holds the dental securement 310 firmly in place. Herein, the dental securement may also be referred to as a dental anchor.

The oral studs 320 may correspond to the support studs 220 and the connectors 330 may correspond to connectors 230, discussed above in connection with FIGS. 2A-2I. As illustrated in FIGS. 3A-3D, the oral stud 320, dental securement 310, and elastic connector 330 may dynamically support and/or retract the tongue 112 thereby maintaining an open passage through the oropharynx 115.

FIG. 3A illustrates an embodiment including one oral stud 320a, a dental securement 310a, and a single connector 330a. As shown in FIG. 3A, the oral stud 320a may be inserted through the tongue 112 or tongue base 113 at a midline position of the tongue 112 or tongue base 113. For example, the oral stud 320a may be inserted at an anterior, midline location of the tongue 112, avoiding nerves, arteries, and taste buds of the tongue 112. The oral stud 320a and the dental securement 310a may be connected to one another with a connector 330a external to the soft tissue of the tongue 112 or tongue base 113. The connector 330a may be used to alter the position of the tongue 112 and, in particular, move the tongue 112 anteriorly away from the oropharynx 115. In the embodiment illustrated by FIG. 3A, the tongue 112 may be shifted slightly forward in the oral cavity 111.

FIG. 3B illustrates an embodiment including two oral studs 320b, a dental securement 310b, and multiple connectors 330b. As shown in FIG. 3B, the oral studs 320b may be inserted through the tongue 112 or tongue base 113, with one on either side of a midline of the tongue 112 or tongue base 113. The oral studs 320b and the dental securement 310b may be connected to one another with connectors 330b external to the soft tissue of the tongue 112 or tongue base 113. The connectors 330b may be used to alter the position of the tongue 112 and, in particular, move the tongue 112 anteriorly away from the oropharynx 115. In the embodiment illustrated by FIG. 3B, the tongue 112 may be shifted slightly forward in the oral cavity 111.

FIG. 3C illustrates an embodiment including one oral stud 320c, two dental securements 310c, and multiple connectors 330c. As shown in FIG. 3C, the oral stud 320c may be inserted through the tongue 112 or tongue base 113 at a midline position of the tongue 112 or tongue base 113. For example, the oral stud 320c may be inserted at an anterior, midline location of the tongue 112, avoiding nerves, arteries, and taste buds of the tongue 112. The oral stud 320c and the dental securements 310c may be connected to one another with connectors 330c external to the soft tissue of the tongue 112 or tongue base 113. In the embodiment of FIG. 3C, a first connector 330c may connect the oral stud 320c with a first one of the dental securements 310c and a second connector 330c may connect the oral stud 320c with a second one of the dental securements 310c. The connectors 330c may be used to alter the position of the tongue 112 and, in particular, move the tongue 112 anteriorly away from the oropharynx 115. In the embodiment illustrated by FIG. 3C, the tongue 112 may be shifted slightly forward in the oral cavity 111.

FIG. 3D illustrates an embodiment including one oral stud 320d, one dental securement 310d, and multiple connectors 330d. As shown in FIG. 3D, the oral stud 320d may be inserted through the tongue 112 or tongue base 113 at a midline position of the tongue 112 or tongue base 113. For example, the oral stud 320d may be inserted at an anterior, midline location of the tongue 112, avoiding nerves, arteries, and taste buds of the tongue 112 The oral stud 320d and the dental securement 310d may be connected to one another with connectors 330d external to the soft tissue of the tongue 112 or tongue base 113. In the embodiment of FIG. 3D, a first connector 330d may connect the oral stud 320d with one end of the dental securement 310d and a second connector 330d may connect the oral stud 320d with the other, distal end of the dental securement 310d. The connector 230 may be used to alter the position of the tongue 112 and, in particular, move the tongue 112 anteriorly away from the oropharynx 115. In the embodiment illustrated by FIG. 3D, the tongue 112 may be shifted slightly forward in the oral cavity 111.

FIGS. 3E-3H are front views of a human head to illustrate placement of components of a multi-component device used in suspension uvulopalatopexy, consistent with certain exemplary embodiments. In the embodiments illustrated by FIGS. 3E-3H, the multi-component device includes at least one oral stud 320 inserted into a uvula 104 or soft palate 102, a securement to the maxilla 119 by attachment or insertion to a maxillary structure that provides support (e.g., teeth or bones), and one or more external elastic connectors 330 that mechanically couple at least one oral stud 320 to the maxillary securement (e.g., dental securement 310). In the embodiments of FIGS. 3E-3H, the dental securement 310 may be removably attached (e.g., placed over the patient's teeth) or permanently attached (e.g., bonded or glued to the teeth, embedded in bone, etc.), such that the patient's teeth or jaw holds the dental securement 310 firmly in place.

In the embodiments illustrated by FIGS. 3E-3H, the multi-component device includes at least one oral stud 320 inserted into a uvula 104 and/or lateral pharyngeal walls 117, a securement to the mandible 118 by attachment or insertion to a mandible structure that provides support (e.g., teeth or bones), and one or more external elastic connectors 330 that mechanically couple at least one oral stud 320 to the mandible securement (e.g., dental securement 310). The dental securement 310 may be removably attached (e.g., placed over the patient's teeth) or permanently attached (e.g., bonded or glued to the teeth, embedded in bone, etc.), such that the patient's teeth or jaw holds the dental securement 310 firmly in place. The oral studs 320 may correspond to the support studs 220 and the connectors 330 may correspond to connectors 230, discussed above in connection with FIGS. 2A-2I and FIGS. 3A-3D. As illustrated in FIGS. 3E-3H, the oral stud 320, dental securement 310, and elastic connector 330 may dynamically support and/or retract the soft palate 102 and/or the lateral pharyngeal walls 117, thereby maintaining an open passage through the oropharynx 115.

FIG. 3E illustrates an embodiment including one oral stud 320e, a dental securement 310e, and a connector 330e. As shown in FIG. 3E, the oral stud 320e may be inserted through the soft palate 102 or the uvula 104 at a midline of the soft palate 102 or uvula 104. The oral stud 320e and the dental securement 310e may be connected to one another with a connector 330e external to the soft tissue of the palate 103. The connector 330e may be used to alter the position of the uvula 104 and, in particular, move the uvula 104 anteriorly away from the pharynx. In the embodiment illustrated by FIG. 3E, due to the positioning of the dental securement 310e and the oral stud 320e, the uvula 104 may be shifted slightly forward, while maintaining a centered position in the oral cavity 111.

FIG. 3F illustrates an embodiment including one oral stud 320f, a dental securement 310f, and multiple connectors 330f. As shown in FIG. 3F, the oral stud 320f may be inserted through the soft palate 102 or the uvula 104 at a midline of the soft palate 102 or uvula 104. The oral stud 320f and the dental securement 310f may be connected to one another with connectors 330f external to the soft tissue of the palate 103. In the embodiment of FIG. 3F, a first connector 330f may connect the oral stud 320f with one end of the dental securement 310f and a second connector 330f may connect the oral stud 320f with the other, distal end of the dental securement 310f. The connectors 330f may be used to alter the position of the uvula 104 and, in particular, move the uvula 104 anteriorly away from the pharynx. In the embodiment illustrated by FIG. 3F, due to the positioning of the dental securement 310f and the oral stud 320f, the uvula 104 may be shifted slightly forward, while maintaining a centered position in the oral cavity 111.

FIG. 3G illustrates an embodiment including two oral studs 320g, a dental securement 310g, and multiple connectors 330g. As shown in FIG. 3G, the oral studs 320g may be inserted through the lateral pharyngeal walls 117, with one on either side of the soft palate 102. In the embodiment of FIG. 3G, a first connector 330g may connect a first oral stud 320g with one end of the dental securement 310h and a second connector 330g may connect the second oral stud 320g with the other, distal end of the dental securement 310g. The connectors 330g may be used to alter the position of the lateral pharyngeal walls 117 and, in particular, move the lateral pharyngeal walls 117 anteriorly away from the pharynx.

FIG. 3H illustrates an embodiment including two oral studs 320h, a dental securement 310h, and multiple connectors 330h. As shown in FIG. 3H, the oral studs 320h may be inserted through the lateral pharyngeal walls 117, with one on either side of the soft palate 102. In the embodiment of FIG. 3H, a first connector 330h may connect a first oral stud 320h with the dental securement 310h and a second connector 330h may connect the second oral stud 320h with the dental securement 310h. The connectors 330h may be used to alter the position of the lateral pharyngeal walls 117 and, in particular, move the lateral pharyngeal walls 117 anteriorly away from the pharynx.

The embodiments of FIGS. 2A-2I and 3A-3H may support/displace the soft palate 102, the tongue 112, and/or the lateral pharyngeal walls 117, thereby improving breathing while causing minimal interference with speech, breathing, and swallowing. In addition, one or more of the size, location, and number of oral studs, as well as the number, type, and tension-grade of the elastic connectors may be altered to introduce flexibility in the customization to the individual patient, thereby maximizing the likelihood for compliance and efficacy in patients suffering from OSA, upper airway resistance syndrome (UARS), and snoring. A single stud or multiple studs may also be used as cosmetic piercings (and need not be used for snoring/sleep apnea).

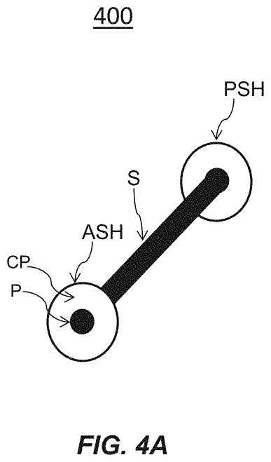

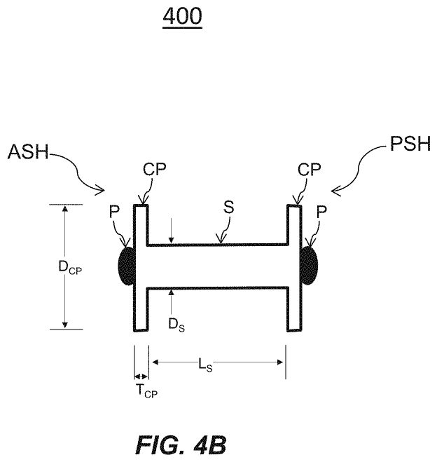

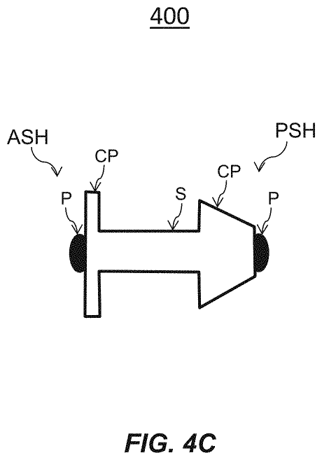

FIGS. 4A and 4B are schematics illustrating an oral stud 400, according to certain exemplary embodiments. As discussed above, the oral stud 400 may be an anchor stud 210 or a support stud 220. FIG. 4A illustrates a perspective view of an oral stud 400, and FIG. 4B illustrates a side view of an oral stud 400, and FIG. 4C illustrates a side view of an oral stud 400.

The oral stud 400 may include a shaft S, a posterior stud head PSH, and an anterior stud head ASH. In some examples, the oral stud 400 may be formed as a single contiguous integrated piece of the same material, such as a flexible plastic material. The shaft S may consist of a rigid material or semi-rigid material (e.g., a suture). The posterior stud head PSH and the anterior stud head ASH may be located at opposite ends of the shaft S. The posterior stud head PSH may be the end of the oral stud 400 that is inserted through the support site (e.g., the uvula 104, the tongue 112, and/or the lateral pharyngeal walls 117) or the anchor site (e.g., the soft palate 102). The anterior stud head ASH may be the end of the oral stud 400 that is located inside the oral cavity 111. In some embodiments, the posterior stud head PSH and the anterior stud head ASH may include circular plates CP that uniformly extend perpendicularly away from the shaft S. When the shaft S of the oral stud 400 consists of a suture, the posterior stud head PSH and the anterior stud head ASH may be held to the suture by one or more stitches. In embodiments including a suture, the suture may be customized for the needs of individual patients at the time of installation. For example, a length of the suture may be determined at the time of insertion. The suture may be placed using a reverse Seldinger technique, which may allow for the suture to be inserted in an office setting and provide for the suture to be removed.

The circular plate CP of each of the posterior stud head PSH and the anterior stud head ASH may have a first side that has a flat or planar shape. The first side may be the side of the circular plate CP nearest the shaft S. For example, the first side may be on the side adjacent to the tissue through which the oral stud 400 is to be inserted. The circular plate CP may have a diameter D_{CP} and a thickness T_{CP}. The diameter D_{CP} of the circular plate CP may be in the range of, for example, several millimeters (e.g., 2-7 millimeters, or more particularly, 3-5 millimeters), and the thickness T_{CP} of the circular plate CP may be in the range of, for example, one or more millimeters (e.g., 1-7 millimeters, or more particularly, 2-3 millimeters). The diameter D_{CP} of the circular plate CP may be larger than a diameter D_{S} of the shaft S. Although the posterior stud head PSH and the anterior stud head ASH are described as plates having a circular shape, it is envisioned that the plates may be formed to have other shapes (e.g., square, rectangular, triangular, pentagonal, etc.). In some cases, the shape of the plates may be determined based on the placement location. For example, a rectangular plate may be used for locations that are narrower or have an elongated shape (e.g., the lateral pharyngeal walls 117).

The shaft S (or suture) may be the portion of the oral stud 400 that is located within tissue of the target (anchor) site and/or the support site. For example, when the oral stud 400 is inserted through the uvula 102, the posterior stud head PSH may be located external to the uvula 104 in the nasopharynx 114, the anterior stud head ASH may be located external to the uvula 104 in the oral cavity 111, and the shaft S (or suture) may extend through the tissue of the uvula 102 between the oral cavity 111 and the nasopharynx 114. As another example, when the oral stud 400 is inserted through the soft palate 102, the posterior stud head PSH may be located external to the soft palate 102 in the nasopharynx 114, the anterior stud head ASH may be located external to the soft palate 102 in the oral cavity 111, and the shaft S (or suture) may extend through the tissue of the soft palate 102 between the oral cavity 111 and the nasopharynx 114. As a further example, when the oral stud 400 is inserted through the lateral pharyngeal walls 117, the posterior stud head PSH may be located external to the lateral pharyngeal walls 117 in the oropharynx 115, the anterior stud head ASH may be located external to the lateral pharyngeal walls 117 in the oral cavity 111, and the shaft S (or suture) may extend through the interior of the lateral pharyngeal walls 117 between the oral cavity 111 and the oropharynx 115.

The shaft S (or suture) may have a length L_{S} corresponding to the length of the hole created in the target (anchor) or support site. For example, the length L_{S} of the shaft S may be such that the shaft S is almost entirely contained within tissue of the target (anchor) or support site. A length L_{S} of the shaft S may correspond to a thickness of the region into which the shaft S is inserted, and may be in the range of, for example, several millimeters to several centimeters. In some embodiments, the length of the shaft S may be 1-2 millimeters longer than the thickness of the region into which the shaft S is inserted. The shaft S may be a cylinder shape and have a diameter D_{S} in the range of, for example, one or more millimeters (e.g., 1-7 millimeters, or more particularly, 2-3 millimeters). The diameter D_{S} of the shaft S may be proportional to its length L_{S}. For example, a shaft S having a greater length L_{S} may also have a larger diameter D_{S}, whereas a shaft S having a shorter length L_{S} may have a smaller diameter D_{S}. In addition, a diameter D_{S} of the shaft S may be determined such that the shaft S, while maintaining flexibility, does not distend or stretch to a greater length.

As shown in FIGS. 4B and 4C, in some embodiments, the posterior stud head PSH may include a plate that is collapsible in one direction (e.g., collapsing toward the central axis of the shaft S and toward the body of the stud, such as away from the insertion direction) to facilitate insertion through the target (anchor) or support site, but resists collapsing in the other direction (e.g., does not collapse toward the axis of the stud away from the body of the stud, such as towards the insertion direction) so that the plate spreads in an uncollapsed position against the surface of the target (anchor) or support site to prevent the oral stud 400 from being extracted through the hole in the target (anchor) or support site, thus keeping the oral stud 400 in place. In FIG. 4B, the circular plate CP may have a rectangular shape, when viewed from the side. In FIG. 4C, the circular plate CP may have a trapezoidal shape (e.g., an isosceles trapezoidal shape), when viewed from the side.

The oral stud 400 may be made of a biocompatible material suitable for long-term implantation within the human body, such as, for example, a metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof. The oral stud 400 may be formed of shape memory materials (SMMs), which are featured by their ability to recover their original shape from a significant and seemingly plastic deformation when a particular stimulus is applied (e.g., superelasticity, visco-elasticity). The ability to return to their original shape is known as the shape memory effect.

In certain embodiments, the oral stud 400 may be comprised of a silicone or plastic material. When made of silicone or plastic, the oral stud is lightweight to help avoid irritation. The light weight also may help in allowing the oral stud 400 to be expelled (by coughing, e.g.) in the event it is dislodged and falls into the airway. The oral stud 400 may also be easily removed (e.g., by clipping the shaft S) in the event the oral stud 400 becomes uncomfortable or the patient's tissue becomes irritated or infected. The thickness and/or material strength of the shaft S may be such that the shaft S may be cut by hand, using a hand-held, mechanical device (e.g., clippers). The material of the shaft S may consist of a rigid material or a semi-rigid material (e.g., a suture).

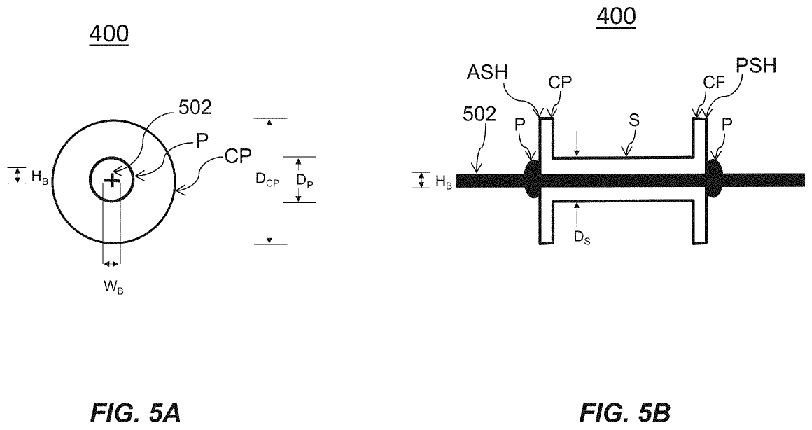

FIGS. 5A and 5B are schematics illustrating oral studs 400 with blades 502 extending through a central axis, according to exemplary embodiments. In some embodiments, the oral stud 400 may include a passageway along a central axis of the shaft S, extending from the posterior stud head PSH to the anterior stud head ASH. The passageway may allow for extension and retraction of a blade 502 along the hollow center. In some embodiments, the passageway may correspond to the size and shape of the blade 502. The blade 502 may be used to pierce the target (anchor) and/or support site, thereby allowing insertion and placement of an oral stud 400.

FIG. 5A is a front view of an oral stud 400 with the blade 502 fully extended through the oral stud 400, and FIG. 5B is a side view of the oral stud 400 with the blade 502 fully extended along the central passageway of the oral stud 400. As shown in the embodiments of FIGS. 5A and 5B, both the height H_{B} and the width W_{B} of the blade 502 may be smaller than the diameter D_{P} of the projection P and the diameter D_{S} of the shaft S. The height H_{B} and the width W_{B} of the blade 502 may be the same as or different from one another. Although not illustrated, in some embodiments, the H_{B} and the width W_{B} of the blade 502 may be the same and may correspond to a diameter of the blade 502.

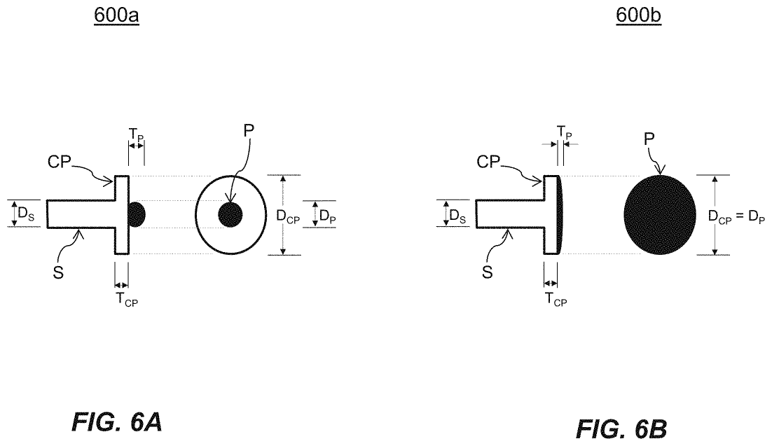

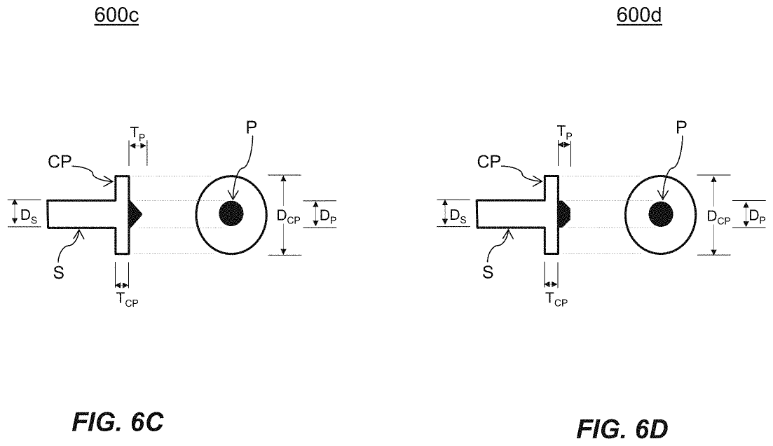

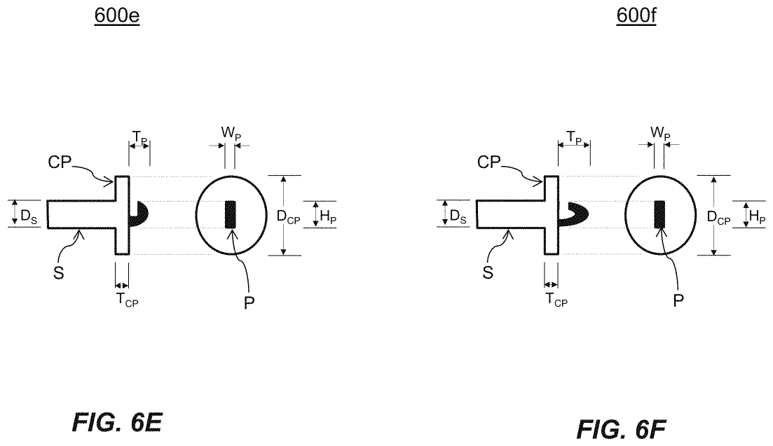

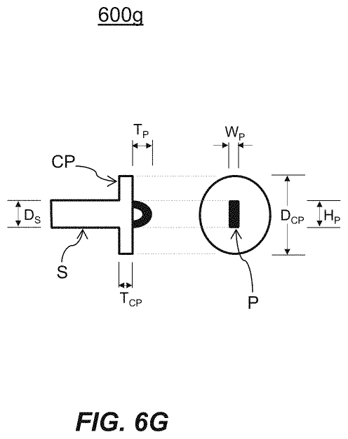

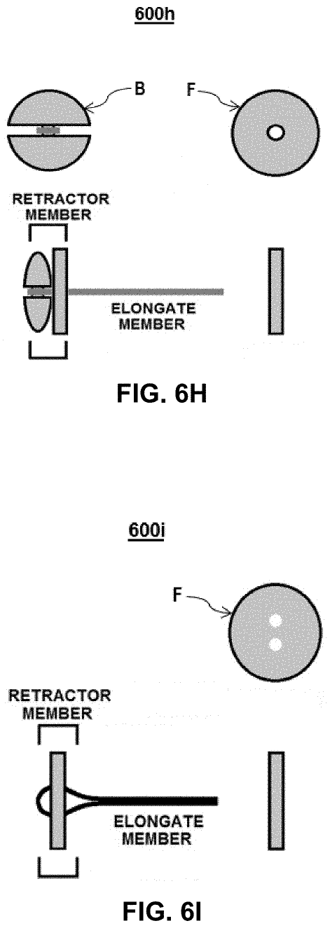

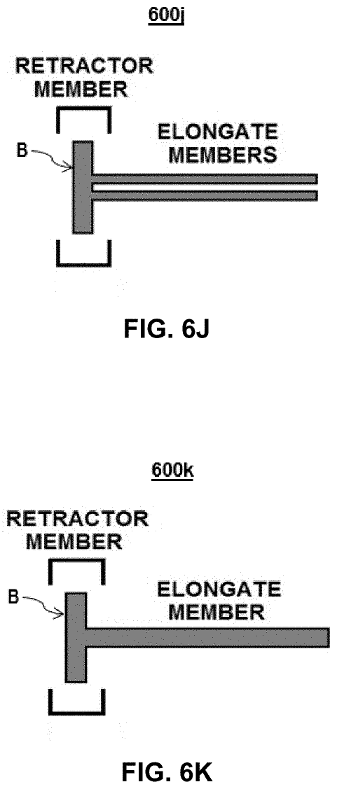

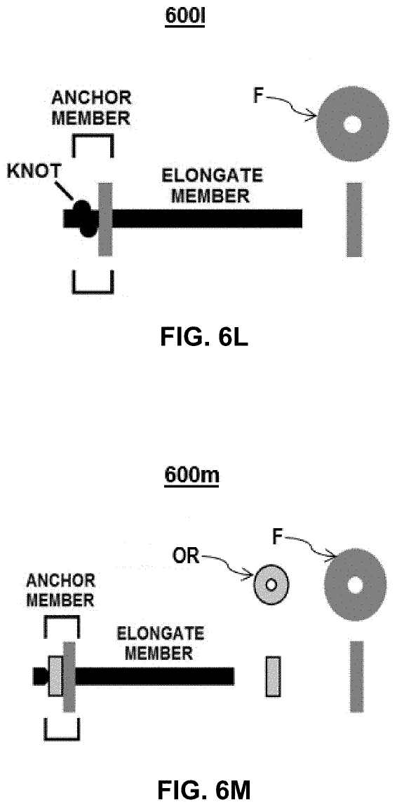

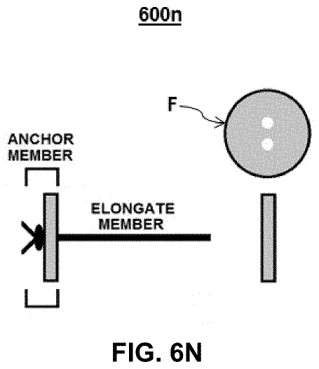

FIGS. 6A-6N are schematics illustrating stud heads 600a through 600n, respectively, according to certain exemplary embodiments. The stud heads 600a through 600n may correspond to the posterior stud head PSH and/or the anterior stud head ASH of FIGS. 5A and 5B above. FIGS. 6A-6G illustrate example embodiments in which the shaft S is a hollow shaft. FIGS. 6H-6N illustrate example embodiments in which the shaft S is a suture or elastic connector. As used herein, the posterior stud head PSH may also be referred to as a “retractor member,” the anterior stud head ASH may also be referred to as an “anchor member,” and the shaft S, suture, or elastic connector may also be referred to as an “elongate member.”

In the embodiments of FIGS. 6A-6G, each of the stud heads 600a through 600g may include a circular plate CP. In some embodiments, the plate CP may have a circle shape, but the embodiments are not limited thereto. For example, the plate CP may have an oval shape, an elongated shape, or a polygonal shape (e.g., a triangle shape, a square shape, a rectangle shape, a rhomboid shape, etc.). The plate CP may have a first side facing the shaft S and a second side, opposite to the first side and facing away from the shaft S. The first side may have a flat or planar shape, and the second side may include a projection P. The projection P may be formed on a top surface of the second side of the circular plate CP to project in a direction away from the shaft S. As illustrated in FIGS. 6A-6G, the projection P may have a variety of shapes, such as, for example, a knob shape (FIG. 6A), a bump shape (FIG. 6B), a sharp or pointed pyramidal shape (FIG. 6C), a rounded pyramidal shape (FIG. 6D), a rounded notched shape (FIG. 6E), a hook shape (FIG. 6F), or a loop shape (FIG. 6G).

When the shape of the projection P, when viewed face-on, is rounded (e.g., FIGS. 6A-6G), the projection P may have a diameter D_{P} and a thickness T_{P}. The diameter D_{P} of the projection P may be in the range of, for example, one or more millimeters (e.g., 1-7 millimeters, or more particularly, 2-3 millimeters), and the thickness T_{P} of the projection P may be in the range of, for example, one or more millimeters (e.g., 1-7 millimeters, or more particularly, 2-3 millimeters). In some embodiments, the diameter D_{P} of the projection P may be the same as or different from a diameter D_{S} of the shaft S. For example, in the embodiments of FIGS. 6A, 6C, 6D, and 6E, the diameters D_{P} of the projections P are the same as the diameters Dₛ of the respective shafts S. In the embodiment of FIG. 6B, the diameter D_{P} of the projection P may be larger than the diameter D_{S} of the shaft S, and may be the same as the diameter D_{CP} of the circular plate CP.

When the shape of the projection, when viewed face-on, is irregular (e.g., FIG. 6F or FIG. 6G), the projection P may have a height H_{P}, a width W_{P}, and a thickness T_{P}. The height H_{P} of the projection P may be in the range of, for example, one or more millimeters (e.g., 1-7 millimeters, or more particularly, 2-3 millimeters), the width W_{P} of the projection P may be in the range of, for example, one or more millimeters (e.g., 1-7 millimeters, or more particularly, 2-3 millimeters), and the thickness T_{P} of the projection P may be in the range of, for example, one or more millimeters (e.g., 1-7 millimeters, or more particularly, 2-3 millimeters). In some embodiments, the height H_{P} and/or width W_{P} of the projection P may be the same as or different from a diameter D_{S} of the shaft S. For example, in the embodiments of FIGS. 6F and 6G, the height H_{P} of the projection P is the same as the diameter Dₛ of the shaft S and the width W_{P} of the projection P is smaller than the diameter Dₛ of the shaft S. Further, although not illustrated, when the shape of the projection is irregular, when the blade 502 is extended through the oral stud 400, the projection P may be shifted or tilted to a side, as discussed in more detail below.

In some embodiments, the posterior stud head PSH and the anterior stud head ASH connected to a same shaft S may include projections P having a same or different material, shape, thickness T_{P}, height H_{P}, width W_{P}, and/or diameter D_{P}. In some embodiments, for example, the posterior stud head PSH may have a smaller thickness T_{P} and larger diameter D_{P} than those of the anterior stud head ASH located at the opposite end of the shaft S. The material, shape, thickness TP and/or diameter DP of the projection P may be determined based on the insertion location or the needs of the patient, and whether the projection P is located on the anterior stud head ASH or the posterior stud head PSH. For example, referring to the embodiment of FIG. 6E, the projection P may have a rounded shape with a notch to retain one end of the connector 230, in the embodiment of FIG. 6F, the projection P may have a hook shape to retain one end of the connector 230, and in the embodiment of FIG. 6G, the projection P may have a loop shape including an opening to retain one end of connector 230. In some exemplary embodiments, the projections P illustrated in FIGS. 6E-6G may be located on the anterior stud heads ASH of the oral stud 400.

In some embodiments, one or more of the projections P of the posterior stud head PSH and/or the anterior stud head ASH may be augmented with additional materials or may be comprised of different materials. For example, in some embodiments, the projection P of the posterior stud head PSH, such as those of FIGS. 6E and 6F, may have a biocompatible metal contained within, and surrounded by, the elastic material forming the oral stud 400. By including a metal in this manner, the projection P may have added rigidity, thereby increasing the ability of the projection P to retain a connector 230.

In other embodiments, the projection P may be formed of a metal, such that an end of the connector 230 is retained against the projection P through a magnetic force. For example, in such an embodiment, the projection P may be a rounded shape (e.g., projection P in FIG. 5A), and the connector may be a cup-shaped magnet or magnetized material that retains and at least partially surrounds the projection P.

The diameter D_{S} and length L_{S} of the shaft S, as well as the thicknesses and diameters of features of the posterior stud head PSH and the anterior stud head ASH, may be determined based on a combination of one or more of the following: (1) the physical size and shape of the target (anchor) and/or support sites and the patient's anatomy, (2) a number of the oral studs, (3) an insertion location of the oral studs, and (4) a desired treatment plan or protocol. For example, when only a smaller displacement force is desired, a fewer number of oral studs may be used and/or the oral studs may be smaller in size, and when a larger displacement force is desired, a larger number of oral studs may be used and/or the oral studs may be larger in size. As another example, the D_{S} and/or length L_{S} of the shaft S may be based on parameters of the patient's anatomy and/or treatment protocol. For example, a desired diameter D_{S} and/or length of the shaft S may be determined based on one or more of a desired amount of tension, an amount of collapse of tissue, a thickness and/or volume of the physical structure to be supported or the physical structure providing the support, patient comfort and/or tolerance, etc.

When fully deployed, a size or contact area of the posterior stud head PSH and/or the anterior stud head ASH may be determined so as to distribute force along a greater area of the patient's tissue. For example, with a greater contact area (e.g., the area of the circular plate CP), the pulling forces at a target (anchor) site and/or the support site (e.g., uvula 104, tongue 112, and/or lateral pharyngeal walls 117) may be dispersed across a greater surface area, thereby reducing irritation and/or discomfort to the patient. The posterior stud head PSH and the anterior stud head ASH may have the same or different shapes and sizes. In some embodiments, the posterior stud head PSH and the anterior stud head ASH may have circular plate CP with the same diameter DCP and thickness TCP, but a different shaped projection P. For example, the posterior stud head PSH may have a projection P with a rounded knob shape as in FIG. 6A, and the anterior stud head ASH may have a projection with a rounded notched shape as in FIG. 6E. As another example, the posterior stud head PSH may have a projection P with a bump shape as in FIG. 6B, and the anterior stud head ASH may have a projection with a looped shape as in FIG. 6G.

FIGS. 6H-6N illustrate example embodiments in which the shaft S is a suture or elastic connector. In the embodiment of FIG. 6H, the stud head 600h may include a button B and a flange F having a single hole. In some embodiments, each of the button B and the flange F may have a circular or a rounded shape. The button B and the flange F may be formed of a silicone, semi-rigid or rigid material. The looping portion of the suture or elastic connector may be inserted through a single hole in the flange F and looped around the button B, such that the looping portion falls into grooves on either side of the button B. When the looping portion of the elastic connector is looped around the button B, the button B may be pulled against the flange F, retaining the button B and flange F firmly against the external surface of a targeted tissue area. As illustrated in FIG. 6H, the button B and flange F may form a retractor member.

In the embodiment of FIG. 6I, the stud head 600i may include a button B having two holes. The button B may be formed of a silicone, semi-rigid or rigid material. In some examples, two end portions of a suture or elastic connector may extend through respective holes of the button B. When the looping portion of the elastic connector is looped through the button B, the button may be pulled firmly against the external surface of a targeted tissue area. As illustrated in FIG. 6I, the button B may form a retractor member.

In the embodiment of FIG. 6J, the stud head 600j may include a button formed of silicone with a first and second silicone elongate member extending from the central body of the button. In some examples, the button may be pulled firmly against the external surface of a targeted tissue area. The button B and the first and second elongate members may be in material continuity with one another. As used herein, the term “material continuity” may refer to structures or devices that are formed at the same time and of the same material, without a break in the continuity of the material of which they are formed. As one example, structures or devices that are in “material continuity” may be homogeneous monolithic structures. As illustrated in FIG. 6J, the button B may form a retractor member.

In the embodiment of FIG. 6K, the stud head 600k may include a button B formed of a silicone with a single silicone elongate member extending from the central body of the button. In some examples, the button may be pulled firmly against the external surface of a targeted tissue area. The button B and the elongate member may be in material continuity with one another. As illustrated in FIG. 6K, the button B may form a retractor member.

In the embodiment of FIG. 6L, the stud head 600l may include a flange F having a hole. The flange F may be formed of a silicone. In some examples, the end portion of an elastic connector may be threaded through the hole of the flange F and tied or knotted off, thereby forming the stud head 600l. In some examples, the stud head 600l may be pulled firmly against the external surface of a targeted tissue area. As illustrated in FIG. 6L, the flange F may form an anchor member.

In the embodiment of FIG. 6M, the stud head 600m may include a flange F and an O-ring OR each having a hole. The O-ring OR and flange F may be formed of a silicone material. In some examples, the end portion of an elastic connector may slip through the flange F hole and tight fit through the O-ring OR, thereby forming the stud head 600m. In some embodiments, the end portion of the elastic connector may be retained by a friction fit with the O-ring OR. In some examples, the stud head 600m may be pulled firmly against the external surface of a targeted tissue area. As illustrated in FIG. 6M, the flange F and O-ring OR may form an anchor member.

In the embodiment of FIG. 6N, the stud head 600n may include a flange F having two holes. The flange F may be formed of a silicone. In some examples, two end portions of a suture or elastic connector may extend through respective holes of the flange F. The two end portions of a suture or elastic connector are tied or knotted off, thereby forming the stud head 600n. In some examples, the stud head 600n may be pulled firmly against the external surface of a targeted tissue area. As illustrated in FIG. 6N, the flange F may form an anchor member.

The thicknesses and diameter of button B and/or flange F, may be determined based on a combination of one or more of the following: (1) the physical size and shape of the target (anchor) site and/or support sites and the patient's anatomy, (2) a number of the stud heads, (3) an insertion location of the stud heads, and (4) a desired treatment plan or protocol. For example, when only a smaller displacement force is desired, a fewer number of stud heads may be used and/or the stud heads may be smaller in size, and when a larger displacement force is desired, a larger number of stud heads may be used and/or the stud heads may be larger in size. When fully deployed, a size or contact area of the button B and/or flange F may be determined so as to distribute force along a greater area of the patient's tissue. For example, with a greater contact area (e.g., the area of the button B and/or flange F), the pulling forces at a target (anchor) site and/or the support site (e.g., uvula 104, tongue 112, and/or lateral pharyngeal walls 117) may be dispersed across a greater surface area, thereby reducing irritation and/or discomfort to the patient. The stud heads 600h and 600n may be used in combination with one another or in combination with any of the stud heads 600a-600g.

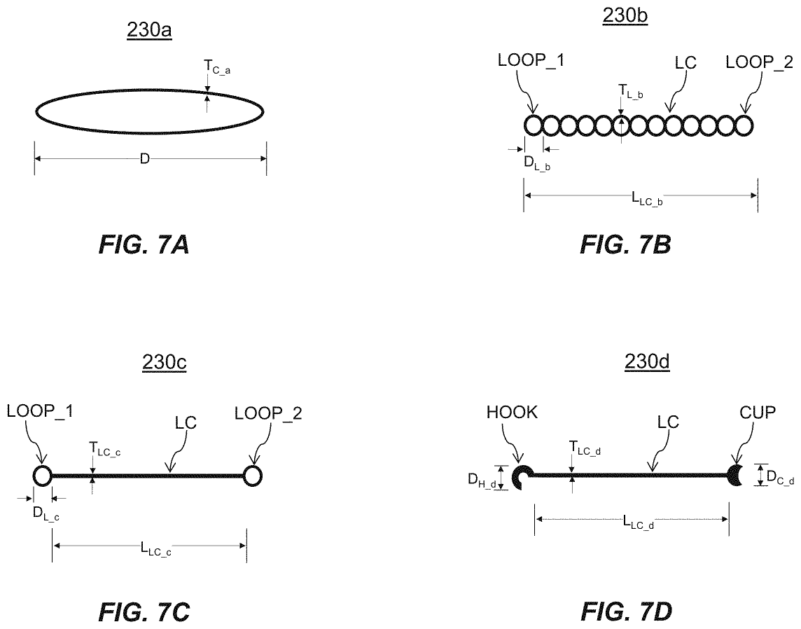

FIGS. 7A-7D are schematics illustrating exemplary connectors, according to certain embodiments. Connectors 230 may be made of a biocompatible material, such as, for example, metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof. The connectors 230 may include a shape memory material (SMM), such that the connector 230 is able to maintain and/or recover its original shape from a significant and seemingly plastic deformation when a particular stimulus is applied (e.g., superelasticity, visco-elasticity). For example, in each of the embodiments of FIGS. 7A-7D, connector 230 may be formed of a material having superelasticity, such that the force of the connector 230 returning to its original shape causes a gentle, continuous pressure to be applied to the anchor studs 210, support studs 220, and dental securements 310 to which it is connected.

In the embodiment of FIG. 7A, connector 230a may be a single continuous loop formed from an elastic band. The continuous loop that forms the connector 230a may attach to an anchor stud 210 (or dental securement 310) and/or a support stud 220 via anterior stud heads ASH having a shape that retains the connector 230a (see, e.g., embodiments of FIGS. 6E and 6F). Connector 230a may be formed of an elastic material that applies a gentle pressure to the anchor stud 210 (or dental securement 310) and support stud 220 to which it is connected. The elastic band that forms the connector 230a may have a thickness T_{C_a} of, for example, approximately one or more millimeters (e.g., 1-7 millimeters, or more particularly, 2-3 millimeters), and a circumferential length CL_{C_a} of, for example, many millimeters (e.g., 10-200 millimeters, or more particularly, 100-150 millimeters). The thickness T_{C_a} and/or the circumferential length CL_{C_a} of the connector 230a may be determined based on a distance between the anchor stud 210 (or dental securement 310) and the support stud 220 to which it is connected, as well as an amount of pressure that is to be applied to the anchor stud 210 (or dental securement 310) and the support stud 220, and an elasticity of the material forming the connector 230a.

In the embodiment of FIG. 7B, connector 230b may be a series of small interconnected loops, and may be comprised of an elastic or rubber material. A first loop LOOP_1 of the series of loops that form the connector 230b may attach to an anchor stud 210 or dental securement 310 having a shape that retains the connector 230b (see, e.g., embodiments of FIGS. 6E and 6F), and a second loop LOOP_2 may attach to a support stud 220 having a shape that retains the connector 230b (see, e.g., embodiments of FIGS. 6E and 6F). There may be one or more third loops located between the first loop LOOP_1 and the second loop LOOP_2. The number of third loops may correspond to a length component LC of the connector 230b, where the length L_{LC_b} of the connector 230b is the end-to-end length of the connector 230b when it is not extended. The material that forms the connector 230 may have a thickness T_{L_b} of, for example, approximately one or more millimeters (e.g., 1-7 millimeters, or more particularly, 2-3 millimeters), and each loop may have a diameter D_{L_b} of, for example, several millimeters (e.g., 3-7 millimeters, or more particularly, 4-5 millimeters). The thickness T_{L_b}, the loop diameter D_{L_b}, and/or the number of loops of the connector 230b may be determined based on a distance between the anchor stud 210 (or dental securement 310) and the support stud 220 to which it is connected, as well as an amount of pressure that is to be applied to the anchor stud 210 (or dental securement 310) and the support stud 220, and an elasticity of the material forming the connector 230b.

In the embodiment of FIG. 7C, connector 230c may consist of two loops LOOP_1 and LOOP_2, connected with one another by a linear component LC. The two loops LOOP_1 and LOOP_2 and the linear component LC may be comprised of an elastic or rubber material. One loop LOOP_1 of the connector 230c may attach to an anchor stud 210 (or dental securement 310) having a shape that retains the connector 230c (see, e.g., embodiments of FIGS. 6E and 6F), and a second loop LOOP_2 of the connector 230c may attach to a support stud 220 having a shape that retains the connector 230c (see, e.g., embodiments of FIGS. 6E and 6F). The linear component LC may attach the first loop LOOP_1 to the second LOOP_2, and may have a length L_{LC_c} measured from the first loop LOOP_1 to the second LOOP_2. The length L_{LC_c} of the linear component LC may be in the range of, for example, several millimeters (e.g., 25-300 millimeters, or more particularly, 50-125 millimeters). The material that forms the connector 230 may have a thickness T_{LC_c} of, for example, one or more millimeters (e.g., 1-7 millimeters, or more particularly, 2-3 millimeters), and each loop may have a diameter D_{L_c} of, for example, several millimeters (e.g., 3-7 millimeters, or more particularly, 4-5 millimeters). The thickness T_{LC_c}, the loop diameter D_{L_c}, and/or the length L_{LC_c} of the linear component LC of the connector 230c may be determined based on a distance between the anchor stud 210 (or dental securement 310) and the support stud 220 to which it is connected, as well as an amount of pressure that is to be applied to the anchor stud 210 (or dental securement 310) and the support stud 220, and an elasticity of the material forming the connector 230c.

In the embodiment of FIG. 7D, connector 230d may consist of a cup CUP and a hook HOOK, connected with one another by a linear component LC. The cup CUP and a hook HOOK may be comprised of a first rigid material (e.g., a metal), and the linear component LC may be comprised of an elastic material. The cup CUP of the connector 230d may attach to an anchor stud 210 (or dental securement 310) having a shape that fits within the cup CUP (see, e.g., embodiment of FIG. 6A), and the hook HOOK of the connector 230d may attach to a support stud 220 having a shape that retains the connector 230d (see, e.g., embodiments of FIG. 6G). The linear component LC may attach the cup CUP to the hook HOOK, and may have a length L_{Lc_d} measured from the cup CUP to the hook HOOK. The length L_{Lc_d} of the linear component LC may be in the range of, for example, several millimeters (e.g., 3-7 millimeters, or more particularly, 4-5 millimeters). The material that forms the connector 230d may have a thickness T_{Lc_d} of, for example, one or more millimeters (e.g., 1-7 millimeters, or more particularly, 2-3 millimeters), the hook HOOK may have a diameter D_{H_d} of, for example, one or more millimeters (e.g., 1-7 millimeters, or more particularly, 2-3 millimeters), and the cup CUP may have a diameter D_{C_d} of one or more millimeters (e.g., 1-7 millimeters, or more particularly, 2-3 millimeters). The thickness T_{LC_d}, and/or the length of the linear component LC of the connector 230d may be determined based on a distance between the anchor stud 210 (or dental securement 310) and the support stud 220 to which it is connected, as well as an amount of pressure that is to be applied to the anchor stud 210 (or dental securement 310) and the support stud 220, and an elasticity of the material forming the connector 230b.

The disclosed embodiments are not limited to those illustrated in FIGS. 7A-7D. Connector 230 may include ends with any combination of a loop LOOP, a cup CUP, or a HOOK. Similarly, the linear component LC may be a single long loop (e.g., linear component LC of FIG. 7A), multiple connected loops (e.g., linear component LC of FIG. 7B), a single linear piece (e.g., linear components LC of FIGS. 7C and 7D), or any combination thereof.

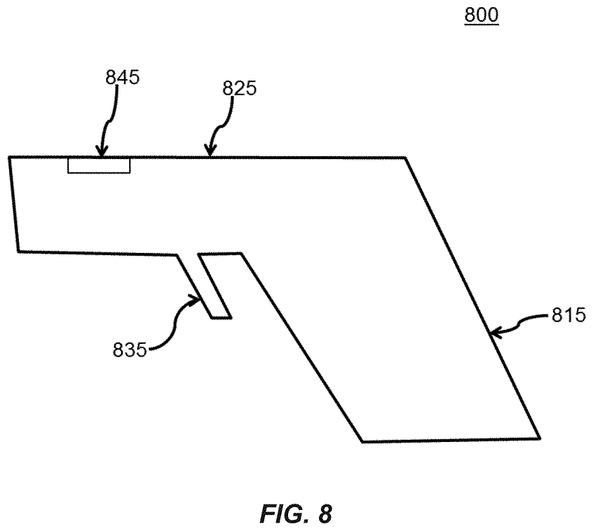

FIG. 8 illustrates an oral stud placement gun 800, according to certain exemplary embodiments. As shown in FIG. 8, an oral stud placement gun 800 may include a handle 815, a barrel 825, a trigger 835, and a load receptacle 835. Although not illustrated in FIG. 8, the oral stud placement gun 800 may also include a blade, blade movement mechanisms, and a suction mechanism. In some embodiments, an oral stud 400 may be loaded in the barrel 825 of the oral stud placement gun 800 through a load receptacle 845. The load receptacle 845 may be an opening in the top of the barrel 825 of a sufficient shape and size sufficient to place an oral stud 400 into the barrel 825. In other embodiments, an oral stud 400 may be loaded in the gun 800 via the front end of the barrel 825. In such an embodiment, the oral stud 400 may be placed in the front end of the barrel 825 of the gun 800 and pressed in the direction of the handle 815.

As discussed further below, the oral stud placement gun 800 may provide for suction using the suction mechanism, to draw a patient's tissue against the end of the barrel 825, and hold the patient's tissue firmly against the end of the barrel 825. When the handle 815 is held in the palm of the user's hand and the user applies pressure to the trigger 835, the blade movement mechanism may begin execution, causing the blade to move through the barrel 825 in a direction from the handle 815 toward the end of the barrel 825. As the blade advances through the barrel 825, it may pass through the center of the oral stud 400 loaded in the barrel 825, and push the oral stud 400 forward out of the end of the barrel 825 into the anchor site or support site.

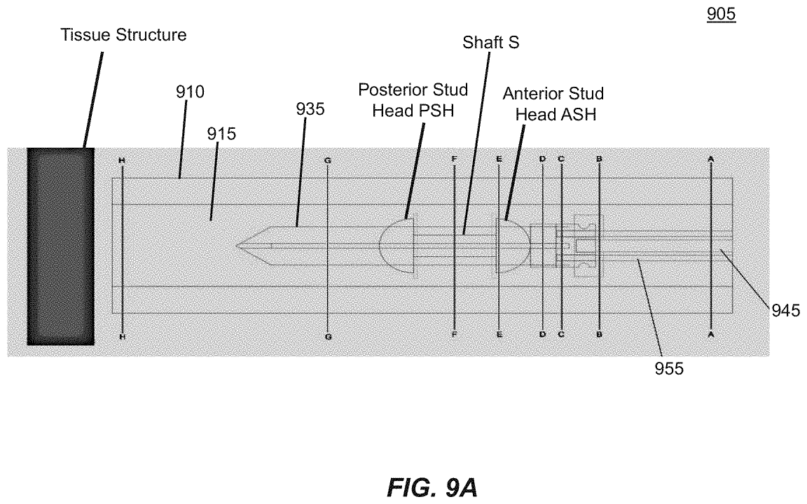

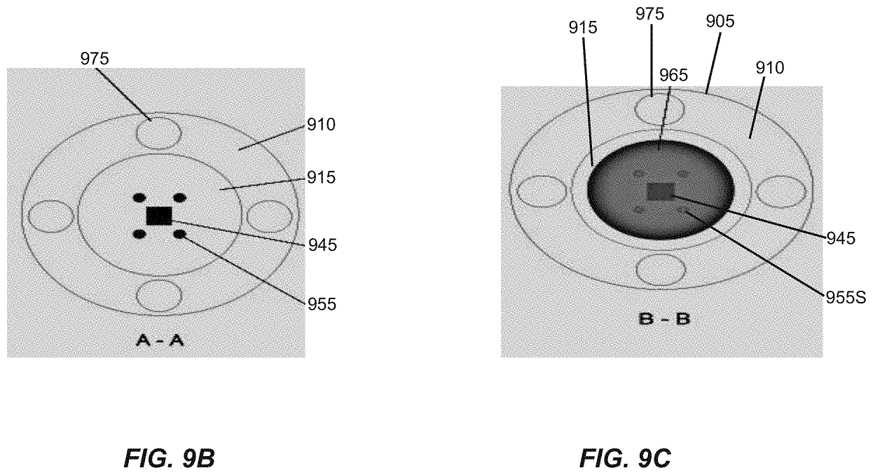

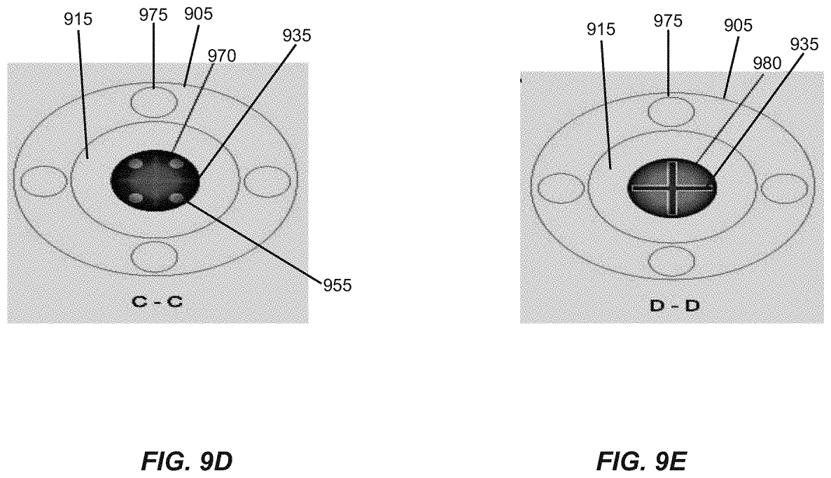

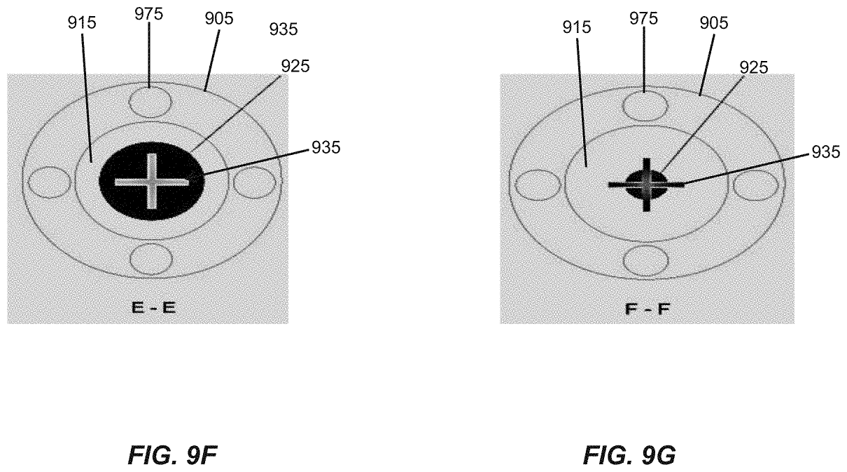

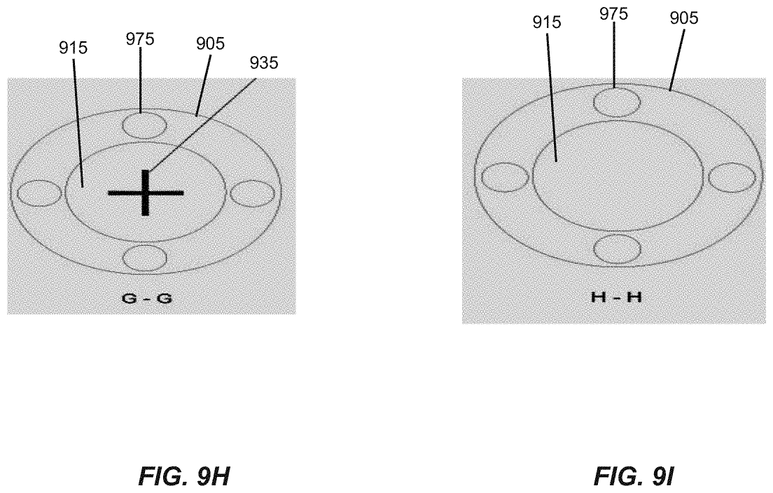

FIG. 9A is a schematic illustrating the arrangement of a blade and oral stud loaded in a barrel of an oral stud placement gun, such as the oral stud placement gun 800 of FIG. 8, according to certain exemplary embodiments. FIGS. 9B-91 are schematics illustrating a blown up views of cross-sections A-A, B-B, C-C, D-D, E-E, F-F, G-G, and H-H, respectively, of FIG. 9A, according to some exemplary embodiments.

Referring to FIG. 9A, an oral stud placement gun may include a barrel 905 having a hollow cylinder 915 surrounded by a housing 910. In the embodiment of FIG. 9A, the barrel 905 may be round, and the hollow cylinder 915 and housing 910 may be concentrically placed along a central axis of the barrel 905. At a rear portion, the barrel 905 may further include a blade drive shaft 945 and a plurality of stud drive shafts 955. When the barrel 905 is loaded with an oral stud 925, the blade drive shaft 945 and plurality of stud drive shafts 955 may be adjacent to the anterior stud head ASH, which may be the accessible portion of the stud for the connector 230 that is not projected through the patient's tissue. The oral stud 925 may be an anchor stud 210 or a support stud 220.

As shown in FIG. 9B, which is a cross-section along line A-A of FIG. 9A, the housing 910 may include a plurality of suction holes 975 (e.g., four). The suction holes 975 may be used to provide a suction force when the barrel 905 is pressed against a target (anchor) site or a support site. In the embodiment of FIG. 9B, the blade drive shaft 945 may be located along a central axis of the barrel 905, and may be surrounded be the plurality of stud drive shafts 955. The stud drive shafts 955 may be placed at equal distances from the blade drive shaft 945 and each other.

As shown in FIGS. 9C and 9D, which are cross-section along lines B-B and C-C, respectively, of FIG. 9A, a blade hub holding member 965 may be provided to hold a blade hub 970. The blade hub holding member 965 and the blade drive shaft 945 may be mechanically mated to one another such that the blade hub holding member 965 and the blade drive shaft 945 move as one unit. The blade hub holding member 965 may be formed to substantially fill the hollow cylinder 915 of the barrel 905. For example, the blade hub holding member 965 may have a diameter than is slightly smaller than the interior diameter of the hollow cylinder 915, such that the edges of blade hub holding member 965 nearly touch the hollow cylinder 915 along the circumference of the blade hub holding member 965, thereby allowing the blade hub holding member 965 to move unimpeded through the hollow cylinder 915. As shown in FIG. 9D, the blade hub 970 may include several cavities 955S that allow each of the corresponding stud drive shafts 955 to move separately from the blade drive shaft 945. The cavities 955S may be empty spaces (e.g., hollow tubes) through which the stud drive shafts 955 advance forward and backward. The blade hub 970 may hold the blade 935, and may control the extension and retraction of the blade 935. The blade hub holding member 965 and blade hub 970 may be formed of plastic.

As shown in FIG. 9E, which is a cross-section along line D-D of FIG. 9A, a sliding stud displacement member 980. The sliding stud displacement member 980 may be configured to move forward and backward along the central axis of the barrel 905. For example, the sliding stud displacement member 980 may provide pressure against an oral stud 925 loaded in the barrel 905, pushing the oral stud 925 toward and through the target (anchor) or support site. The sliding stud displacement member 980 may have an opening that allows for the blade 935 to move through the sliding stud displacement member 980 and to the oral stud 925.

FIGS. 9F and 9G, which are cross-sections along lines E-E and F-F, respectively, of FIG. 9A, illustrate placement of the blade 935 along a central axis of the oral stud 925. Specifically, FIG. 9F is a cross-section of the blade 935 passing through the anterior stud head ASH of the oral stud 925, and FIG. 9G is a cross-section of the blade 935 passing through the shaft S of the oral stud 925. In the embodiments illustrated by FIGS. 9A-9I, the height H_{B} and width W_{B} of the blade 935 may be smaller than a diameter D_{CP} of a circular plate CP of the anterior stud head ASH, and larger than, the same as, or smaller than a diameter D_{S} of the shaft S of the oral stud 925.

FIG. 9H, which is a cross-section along line G-G of FIG. 9A, illustrates the advancement of the blade 935 through the barrel 905. As shown in FIG. 9H, the blade 935 advances through the barrel 905 ahead of the oral stud 925 to allow for the blade to pierce the target (anchor) or support site, making a hole in the target (anchor) or support site, before the oral stud 925 is advanced through the target (anchor) or support site.

FIG. 9I, which is a cross-section of line H-H of FIG. 9A, illustrates a face-on view of the barrel 905. As shown in FIG. 9I, the suction holes 975 extend through the length of the barrel 905 and are concentrically open to the target (anchor) or support site. For example, when the barrel 905 is centered over and contacts the target (anchor) or support site, a suction force is applied concentrically to the area surrounding target (anchor) or support site, drawing the area around the target (anchor) or support site firmly against the barrel 905. The suction force exerted by the suction holes 975 may create an air-tight seal of the suction holes 975 with the tissue surrounding the target (anchor) or support site, thereby preventing relative movement of the target (anchor) or support site with respect to the barrel 905.

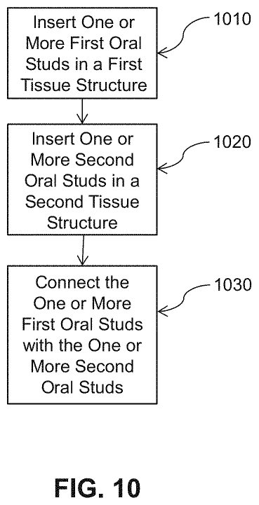

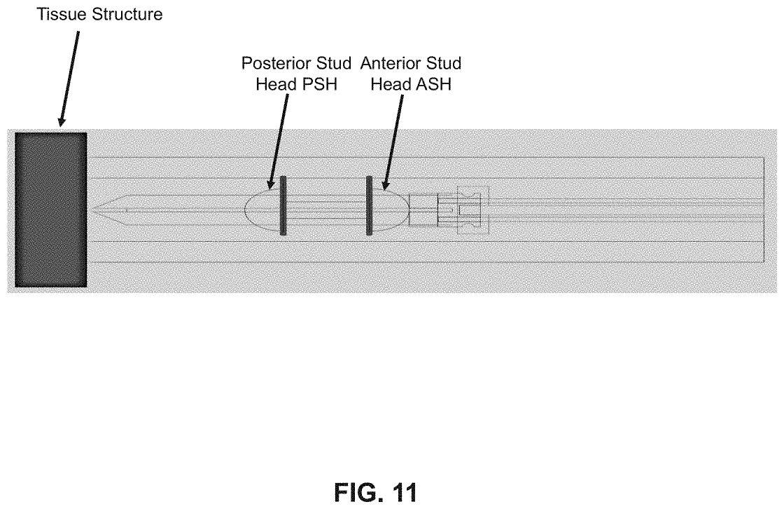

FIG. 10 is a flowchart of a method of suspension uvulopalatopexy using a multi-component device, according to certain exemplary embodiments. FIGS. 11-14 are schematics illustrating the steps of FIG. 10. The systems and methods for suspension uvulopalatopexy, as disclosed and described herein, may include two or more oral studs 925 and one or more elastic connectors 230 (e.g., as illustrated in FIGS. 2A-2G). The oral studs 925 and elastic connectors 230 may work together to affect a position of the soft palate 102, the tongue 112, and/or the lateral pharyngeal walls 117. For example, in some cases, the oral studs 925 and elastic connectors 230 may bring the uvula 104, the tongue 112, and/or lateral pharyngeal walls 117 forward, thereby preventing the air passageway between soft palate 102 and oropharynx 115 from becoming narrow or blocked.

Referring to FIG. 10, one or more first oral studs 925 (e.g., support studs 220) may be inserted into a first tissue structure (e.g., the uvula 103, the tongue 112, and/or the lateral pharyngeal walls 117) (step 1010). The one or more support studs 220 may be inserted using a mechanized device, such as the exemplary oral stud placement gun 800 discussed above.

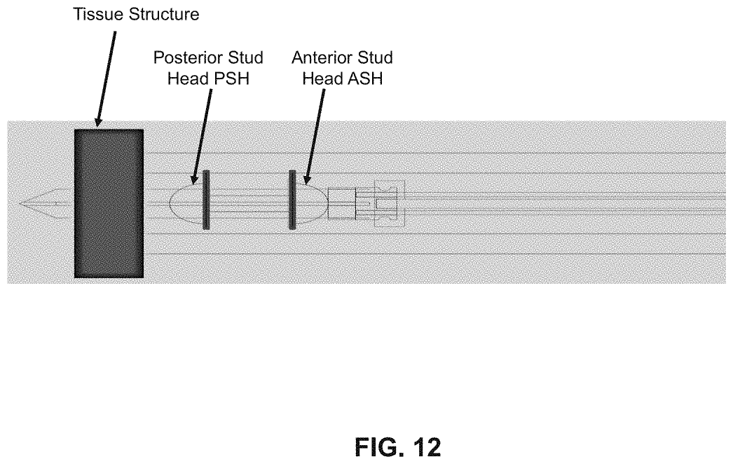

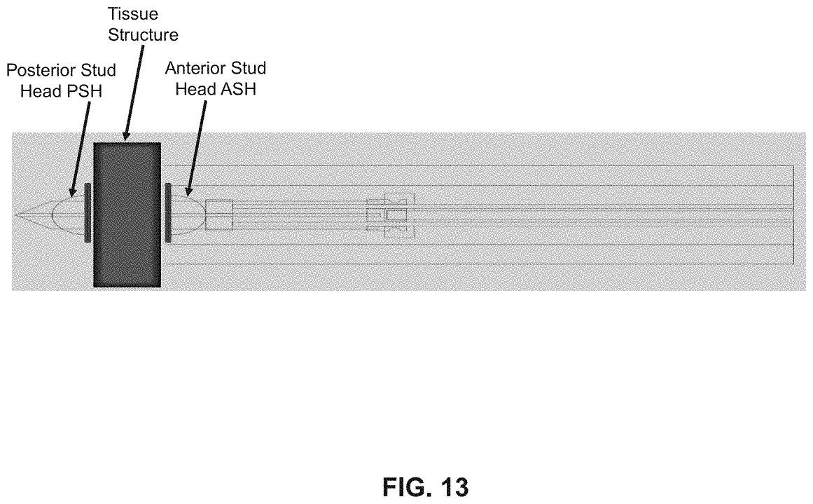

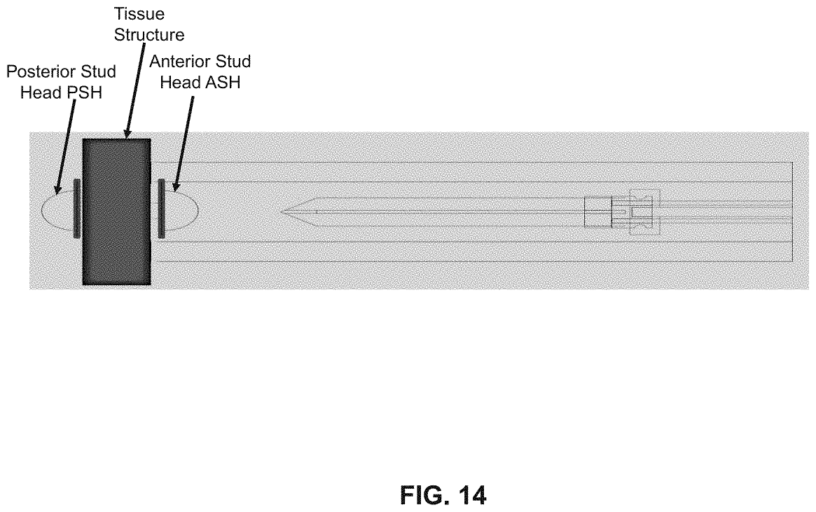

As shown in FIG. 11, when the barrel 905 of the oral stud placement gun 800 is placed in contact with the target (anchor) or support site, the suction holes 975 located in the housing 910 may engage with tissue of the tissue structure, holding the tissue structure firmly against the barrel 905. Then, as shown in FIG. 12, the blade drive shaft 945 may engage, causing the blade 935 to extend through the front portion of the barrel 905, and incise the tissue structure, thereby forming an opening in the tissue structure. Next, as shown in FIG. 13, the stud drive shafts 955 and sliding stud displacement member 980 may engage, causing the oral stud 925 to move through the barrel 905, and advance through the opening in the tissue structure formed by the blade 935. Finally, as shown in FIG. 14, when the oral stud 935 is deployed in the tissue structure, the blade 935 may retract within the barrel 905, allowing for another oral stud 925 to be loaded into the oral stud placement gun 800.

Returning to FIG. 10, one or more second oral studs 925 (e.g., anchor studs 210) may be inserted into a second tissue structure (e.g., the soft palate 102) (step 1020). Similar to step 1010, the one or more oral studs 925 may be inserted using a mechanized device, such as the oral stud placement gun 800. In some embodiments, as reflected in FIGS. 11-14, the mechanized device may be configured to hold the tissue structure, incise the tissue structure, and advance an oral stud 925 into a predetermined location of the tissue structure.

Finally, the one or more first oral studs 925 may be connected to one or more second oral studs 925 via one or more connectors 230 (step 1030). The one or more first and second oral studs 925 are connected with one or more elastic connectors 230 external to the tissue of the tissue structure. In some embodiments, the one or more connectors 230 may be attached to and/or detached from the one or more oral studs 925 by hand (e.g., using one or more fingers to hold and attach/detach the connectors 230) or using a mechanical tool (e.g., an insertion/extraction hook or device). The one or more of the connectors 230 may be replaced in a similar manner. The attachment of the one or more connectors 230 to the one or more oral studs 925 may pull the soft palate 102, the tongue 112, and/or the lateral pharyngeal walls 117 away from the airway to help with snoring and/or sleep apnea. Examples of the connections formed between the one or more first and second oral studs 925 are discussed above in connection with FIGS. 2A-2I and FIGS. 3A-3B. Example connectors 230 are discussed further above in connection with FIGS. 7A-7D.

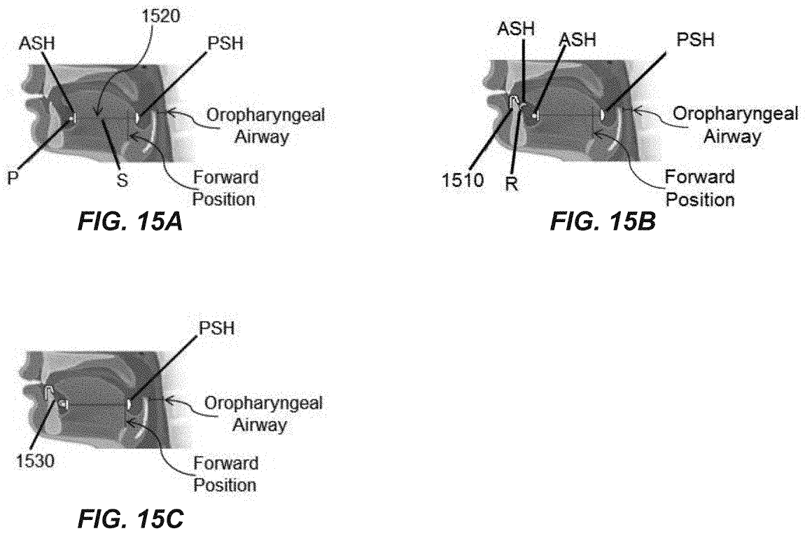

FIGS. 15A-15C are cross-sectional views of a human head to illustrate placement of components of a multi-component device used in suspension glossopexy, consistent with certain exemplary embodiments. Specifically, FIGS. 15A-15C illustrate an embodiment in which an oral stud 1520 is inserted into the tongue 112, bringing the tongue 112 forward in the oral cavity 111 and increasing the space in the oropharynx 115. In the embodiment illustrated by FIGS. 15A-15C, the multi-component device includes one oral stud 1520 inserted into a tongue 112, a dental securement 1510 attached to or inserted into a structure that provides support, and one or more external elastic connectors 1530 that mechanically couple the oral stud 1520 to the dental securement 1510. As illustrated in FIGS. 15A-15C, the oral stud 1520, dental securement 1510, and elastic connector 1530 may maintain a position of, or bring forward, the tongue 112 in the oral cavity 111, thereby maintaining an open passage through the oropharynx 115.

The oral stud 1520 may correspond to the support studs 220 of FIGS. 2A-2I, oral studs 320 of FIGS. 3A-3D, the oral studs 600 of FIGS. 6A-6K, and the connectors 1530 may correspond to connectors 230 of FIGS. 2A-2I and connectors 330 of FIGS. 3A-3D, and the dental securement 1510 may correspond to the dental securements 310 of FIGS. 3A-3D. In the embodiments of FIGS. 15A-15C, the oral stud 1520 includes a shaft S (or elastic elongate member), a posterior stud head PSH, an anterior stud head ASH, and an optional projection P attached to the anterior stud head ASH. As shown in FIG. 15A, the oral stud 1520 may inserted into the tongue 112 at a midline of the tongue 112. The oral stud 1520 may be inserted such that the posterior stud head PSH is projected through the posterior aspect of the tongue 112, passing through the tissue of the tongue 112, to protrude from the posterior aspect of the tongue 112 near the epiglottis 105. When the oral stud 1520 is fully inserted into the tongue, the posterior stud head PSH and the anterior stud head ASH may be external to the tissue of the tongue 112, and the shaft S (or elastic elongate member) may be internal to the tissue of the tongue 112. Referring to FIG. 15B, a dental securement 1510 may be placed in the oral cavity 111. In the embodiment of FIGS. 15A-15C, the dental securement 1510 may be removably attached (e.g., placed over the patient's teeth) or permanently affixed (e.g., glued or bonded to the teeth), such that the patient's teeth hold the dental securement 1510 firmly in place. The dental securement 1510 and the oral stud 1520 may be connected to one another with a connector 1530 external to the tongue 112. In one example embodiment, the projection P, attached to the anterior stud head ASH, and connector R, attached elastically (e.g., via connector 1530) to dental securement 1510, are both formed of ferromagnetic material, and, as such, may magnetically couple to one another in a reversible manner.

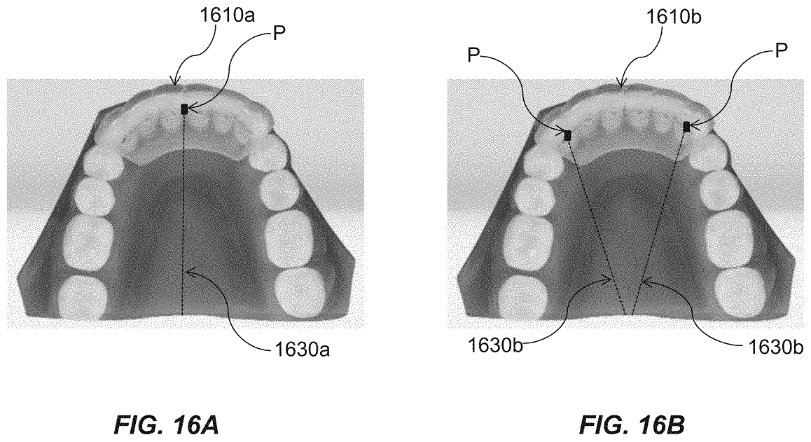

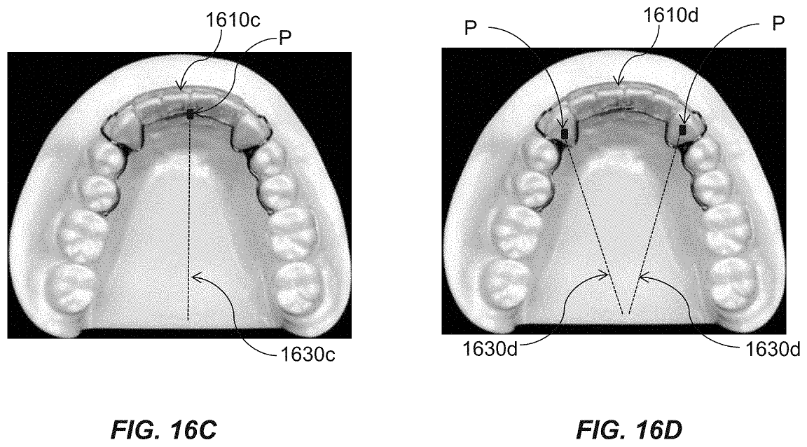

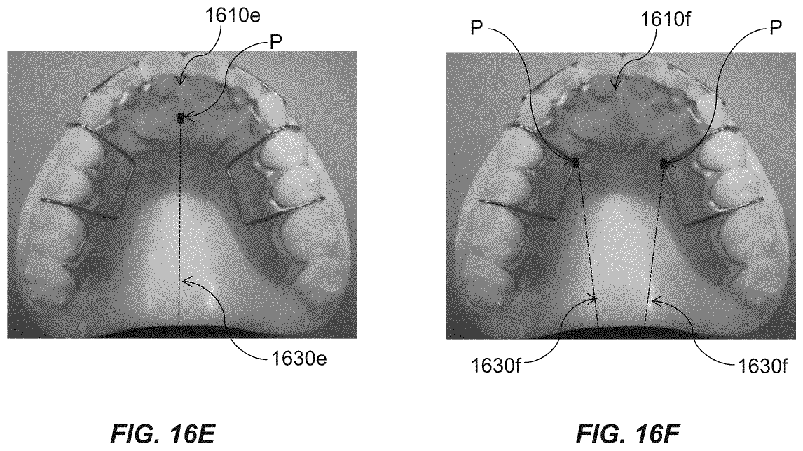

FIGS. 16A-16F are diagrams illustrating placement of certain components of a multi-component device, consistent with certain exemplary embodiments. Specifically, FIGS. 16A-16F illustrate dental securements 1610 that may be removably attached to a patient's teeth. When removably attached, the dental securement 1610 may be inserted and/or removed from the patient's oral cavity 111, as desired. Together with one or more elastic connectors 1630 and one or more oral studs (not shown in FIGS. 16A-16F), dental securements 1610 may dynamically support and/or retract the soft palate 102, the tongue 112, and/or the lateral pharyngeal walls 117. In FIGS. 16A-16F, dental securements 1610 may correspond to dental securements 310 of FIGS. 3A-3H, and connectors 1630 may correspond to connectors 230 of FIGS. 2A-2I and connectors 230 of FIGS. 3A-3H.

In FIGS. 16A-16B, dental securements 1610a and 1610b may be formed of a rigid or semi-rigid material. Dental securements 1610a and 1610b may extend across the patient's teeth (e.g., over the entire arch of teeth, over a portion extending from incisor to incisor, etc.), and may be conformally shaped to the patient's teeth. FIG. 16A illustrates an embodiment having one dental securement 1610a and one connector 1630a, and FIG. 16B illustrates an embodiment having one dental securement 1610b and two connectors 1630b. Although not illustrated, connector 1630a of FIG. 16A may be connected at a distant end of connector 1630a to an oral stud (e.g., oral stud 320a of FIG. 3A or oral stud 320b of FIG. 3B), and connectors 1630b of FIG. 16B may be connected at distant ends of connectors 1630a to an oral stud (e.g., oral stud 320d of FIG. 3D).

In FIGS. 16C-16D, dental securements 1610c-1610d may be formed of a metal wire looped around one or more teeth and anchored in a rigid or semi-rigid material. For example, in FIGS. 16C-16D, dental securements 1610c and 1610d may have a rigid or semi-rigid material extending over the lower front teeth, with metal wires looped around the lower incisors. FIG. 16C illustrates an embodiment having one dental securement 1610c and one connector 1630c, and FIG. 16D illustrates an embodiment having one dental securement 1610d and two connectors 1630d. Although not illustrated, connector 1630c of FIG. 16C may be connected at a distant end of connector 1630c to an oral stud (e.g., oral stud 320a of FIG. 3A or oral stud 320b of FIG. 3B), and connectors 1630d of FIG. 16D may be connected at distant ends of connectors 1630d to an oral stud (e.g., oral stud 320d of FIG. 3D).

In FIGS. 16E-16F, dental securements 1610e-1610f may be formed of a metal wire, which is anchored in an acrylic baseplate that sits in the roof of the mouth near the palate 103. The metal wire may surround one or more teeth, which retains the dental securement 1610. FIG. 16E illustrates an embodiment having one dental securement 1610e and one connector 1630e, and FIG. 16D illustrates an embodiment having one dental securement 1610d and two connectors 1630f. Although not illustrated, connector 1630e of FIG. 16E may be connected at a distant end of connector 1630e to one or more oral studs (e.g., oral stud 320e of FIG. 3E or oral studs 320g of FIG. 3G), and connectors 1630f of FIG. 16F may be connected at distant ends of connectors 1630f to one or more oral studs (e.g., oral stud 320f of FIG. 3F or oral studs 320h of FIG. 3H).

In each of FIGS. 16A-16F, the dental securements 1610 may include a projection P attached to, or formed from, the rigid or semi-rigid material, and the projection P may be configured to retain the connector 1630. For example, the projection P may have a rounded shape with a notch to retain one end of the connector 1630 (e.g., projection P in FIG. 6E), the projection P may have a hook shape to retain one end of the connector 1630 (e.g., projection P in FIG. 6F), or the projection P may have a loop shape including an opening to retain one end of connector 1630 (e.g., projection P in FIG. 6G). In other embodiments, the projection P may be formed of a metal, such that an end of the connector 1630 is retained against the projection P through a magnetic force. For example, in such an embodiment, the projection P may be a rounded shape (e.g., projection P in FIG. 5A), and the connector may be a cup-shaped magnet or magnetized material that retains and at least partially surrounds the projection P.

The dental securements 1610 may be made of a biocompatible material suitable for long-term implantation or use within the human body, such as, for example, a metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof.

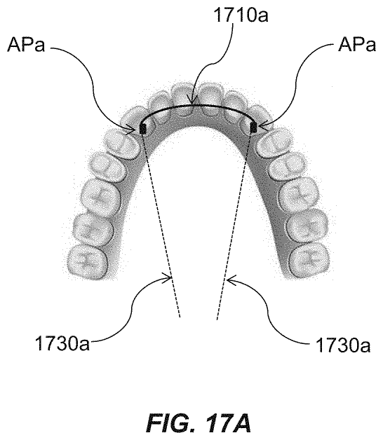

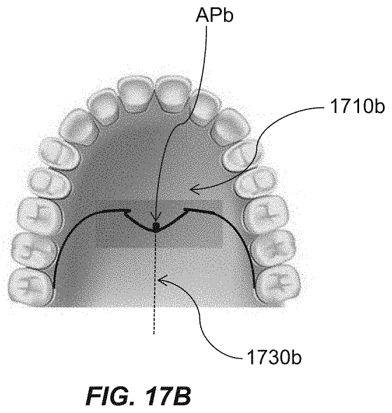

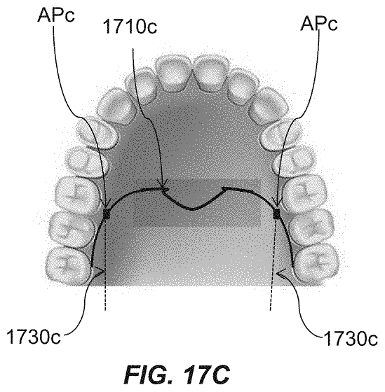

FIGS. 17A-17C are diagrams illustrating placement of certain components of a multi-component device, consistent with certain exemplary embodiments. Specifically, FIGS. 17A-17C illustrate dental securements 1710 that are permanently or semi-permanently attached to a patient's teeth. For example, the dental securement 1710 may be glued or bonded to one or more teeth. Together with one or more elastic connectors 1730 and one or more oral studs (not shown in FIGS. 17A-17C), dental securements 1710 may dynamically support and/or retract the soft palate 102, the tongue 112, and/or the lateral pharyngeal walls 117. In FIGS. 17A-17C, dental securements 1710 may correspond to dental securements 310 of FIGS. 3E-3H, and connectors 1630 may correspond to connectors 230 of FIGS. 2A-2I and connectors 230 of FIGS. 3E-3H.

In FIG. 17A, dental securement 1710a may be formed of a metal wire looped around one or more teeth and anchored to the anterior portion of one or more teeth. For example, in FIG. 17A, dental securement 1710a may include two metal portions that are respectively bonded or glued to the anterior portions of the lower canines, with a metal wire extending behind the teeth between the two canines and connecting the two bonded portions. FIG. 17A illustrates an embodiment having one dental securement 1710a with two points of attachment APa and two connectors 1730a. Although not illustrated, connectors 1730a of FIG. 17A may be connected at distant ends of connectors 1730a to one or more oral studs (e.g., oral stud 320e of FIG. 3E or oral stud 320f of FIG. 3F).

In FIGS. 17B and 17C, dental securements 1710b and 1710c may be formed of a metal wire looped around one or more teeth and anchored around one or more teeth. For example, in FIGS. 17B and 17C, dental securements 1710b and 1710c may include two metal bands that are respectively bonded or glued to rear teeth (e.g., molars), with a metal wire extending between the two metal bands. The shapes of the metal wire may vary and the number of attachment points AP may be determined based on the structures to be supported. FIG. 17B illustrates an embodiment having one dental securement 1710b with one point of attachment APb and one or more connectors 1730b, and FIG. 17C illustrates an embodiment having one dental securement 1710c with two point of attachment APc and one or more connectors 1730c. Although not illustrated, the one or more connectors 1730b of FIG. 17B may be connected at distant ends of connectors 1730b to one or more oral studs (e.g., oral stud 320e of FIG. 3E or oral stud 320g of FIG. 3G), and the one or more connectors 1730c of FIG. 17C may be connected at distant ends of connectors 1730b to one or more oral studs (e.g., oral stud 320f of FIG. 3F or oral stud 320h of FIG. 3H).

In each of FIGS. 17A-17C, the dental securements 1710 may include an attachment point AP. The attachment points AP may be attached to, or formed from, the rigid or semi-rigid material, and the projection P may be configured to retain connectors 1730. The attachment points AP may be the same as, or similar to the projections P. For example, the attachment point AP may have a rounded shape with a notch to retain one end of the connector 1730 (e.g., projection P in FIG. 6E), the attachment point AP may have a hook shape to retain one end of the connector 1730 (e.g., projection P in FIG. 6F), or the attachment point AP may have a loop shape including an opening to retain one end of connector 1730 (e.g., projection P in FIG. 6G). In other embodiments, the attachment point AP may be formed of a metal, such that an end of the connector 1730 is retained against the attachment point AP through a magnetic force. For example, in such an embodiment, the attachment point AP may be a rounded shape (e.g., projection P in FIG. 5A), and the connector may be a cup-shaped magnet or magnetized material that retains and at least partially surrounds the attachment point AP.

The dental securements 1710 may be made of a biocompatible material suitable for long-term implantation or use within the human body, such as, for example, a metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof.

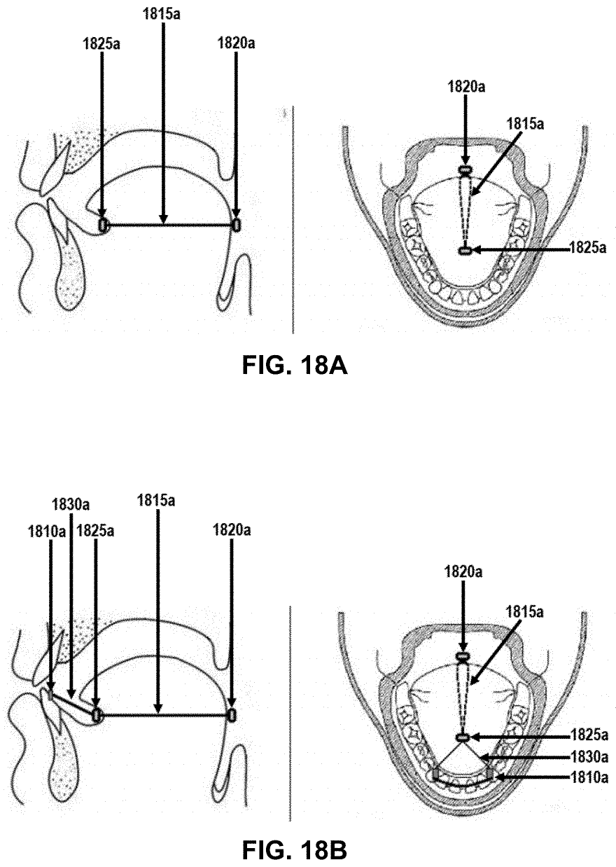

FIGS. 18A-18B, 19A-19B, 20A-20B, 21A-21B, 22A-22B, 23A-23B, 24A-24B, 25A-25B, 26A-26B, and 27A-27B are front and side views of a human head to illustrate placement of components of a multi-component device used in suspension glossopexy, consistent with certain exemplary embodiments.

In the embodiments illustrated by FIGS. 18A-18B, 19A-19B, 20A-20B, 21A-21B, 22A-22B, 23A-23B, 24A-24B, 25A-25B, 26A-26B, and 27A-27B, the multi-component device may include at least one retractor member 1820, at least one anchor member 1825, and at least one elongate member 1815 extending between the at least one retractor member 1820 and the at least one anchor member 1825. FIGS. 18B, 19B, 20B, 21B, 22B, 23B, 24B, 25B, 26B, and 27B further illustrate the multi-component device including a dental securement 1810 attached to or inserted into a structure that provides support (e.g., teeth), and one or more external elastic connectors 1830 that mechanically couple the at least one anchor member 1825 to the dental securement 1810. In the embodiments of FIGS. 18B, 19B, 20B, 21B, 22B, 23B, 24B, 25B, 26B, and 27B, the dental securement 1810 may be removably attached (e.g., placed over the patient's teeth) or permanently attached (e.g., bonded or glued to the teeth, etc.), such that the patient's teeth or jaw holds the dental securement 1810 firmly in place.

In the embodiments illustrated by FIGS. 18A-18B, 19A-19B, 20A-20B, 21A-21B, 22A-22B, 23A-23B, 24A-24B, 25A-25B, 26A-26B, and 27A-27B, the multi-component device may be used to alter the position of the tongue 112 and, in particular, move the tongue 112 anteriorly away from the oropharynx 115. For example, the tongue 112 may be shifted slightly forward in the oral cavity 111.

Each of the at least one retractor members 1820 and the at least one anchor members 1825 may be, for example, the stud heads 600 of FIGS. 6H-6N. The dental securements 1810 may correspond to any of the dental securements 310 of FIGS. 3A-3D. The connectors 1830 may correspond to any of the connectors 230 of FIGS. 2A-21 and/or the connectors 230 of FIGS. 3A-3D.

The at least one elongate member 1815 may be inserted into and extend through a patient's oral tissue, such as, for example, a uvula 104, a tongue 112 or tongue base 113, or lateral pharyngeal walls 117. The at least one elongate member 1815 may be made of a biocompatible material, such as, for example, metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof. The at least one elongate member 1815 may include a shape memory material (SMM), such that the at least one elongate member 1815 is able to maintain and/or recover its original shape from a significant and seemingly plastic deformation when a particular stimulus is applied (e.g., superelasticity, visco-elasticity). For example, in each of the embodiments of FIGS. 18A-18B, 19A-19B, 20A-20B, 21A-21B, 22A-22B, 23A-23B, 24A-24B, 25A-25B, 26A-26B, and 27A-27B, the at least one elongate member 1815 may be formed of a material having superelasticity, such that the force of the at least one elongate member 1815 returning to its original shape causes a gentle, continuous pressure to be applied to the at least one retractor member 1820 and the at least one anchor member 1825 to which the at least one elongate member 1815 is connected. In example embodiments, one or more of the at least one retractor member 1820 and the at least one anchor members 1825 may be provided in the region of the pharynx, a region of the tongue, a region of the mandible bone, or a region of the hyoid bone, as discussed below.

FIGS. 18A and 18B illustrate embodiments in which the multi-component device includes one retractor member 1820a, one anchor member 1825a, and one elongate member 1815a extending between the retractor member 1820a and the anchor member 1825a. In FIG. 18A, the elongate member 1815a may extend from the anchor member 1825a to the retractor member 1820a through the tissue of tongue 112, exit the tissue of the tongue 112 at the base of the tongue 112 in the region of the pharynx, connect to the retractor member 1820a, and then extend from the retractor member 1820a to the anchor member 1825a through the tissue of tongue 112. The elongate member 1815a may be connected to the retractor member 1820a by a loop. For example, when the retractor member 1820a is comprised of stud head 600h, the elongate member 1815a may extend through the hole of a flange F and loop around a button B of stud head 600h of the retractor member 1820a. As another example, when the retractor member 1820a is comprised of stud head 600i, the elongate member 1815a may be looped through the holes of the flange F of stud head 600i of the retractor member 1820a. The first and second ends of the elongate member 1815a may be pulled through the anchor member 1825a to adjust the tension before securement of the first and second ends of the elongate member 1815a to the anchor member 1825a. For example, when the anchor member 1825a is comprised of stud head 600m, the first and second ends of the elongate member 1815a may be slipped through the hole of a flange F of stud head 600m of the anchor member 1825a, adjusted in length, and then forcibly pulled through the hole of an O-ring OR of stud head 600m of the anchor member 1825a that locks the flange F against the tongue 112. As another example, when the anchor member 1825a is comprised of stud head 600n, the first and second ends of the elongate member 1825a may be slipped through the first and second holes of a flange F of stud head 600n of the anchor member 1825a, respectively, and knotted off to lock the flange F against the tongue 112. As shown in FIGS. 18A and 18B, the elongate member 1815a may be inserted through the tongue 112 or tongue base 113 at a midline position of the tongue 112 or tongue base 113. For example, the elongate member 1815a may be inserted at an anterior, midline location of the tongue 112, avoiding nerves, arteries, and taste buds of the tongue 112. In FIG. 18B, the multi-component device may further include a dental securement 1810a and connector 1830a connecting the dental securement 1810a to the anchor member 1825a. The dental securement 1810a may include one or more attachment points, allowing the connector 1830a to attach to the dental securement 1810a. In such embodiments, the anchor member 1825a may also include an attachment member, allowing the connector 1830a to attach to the anchor member 1825a.

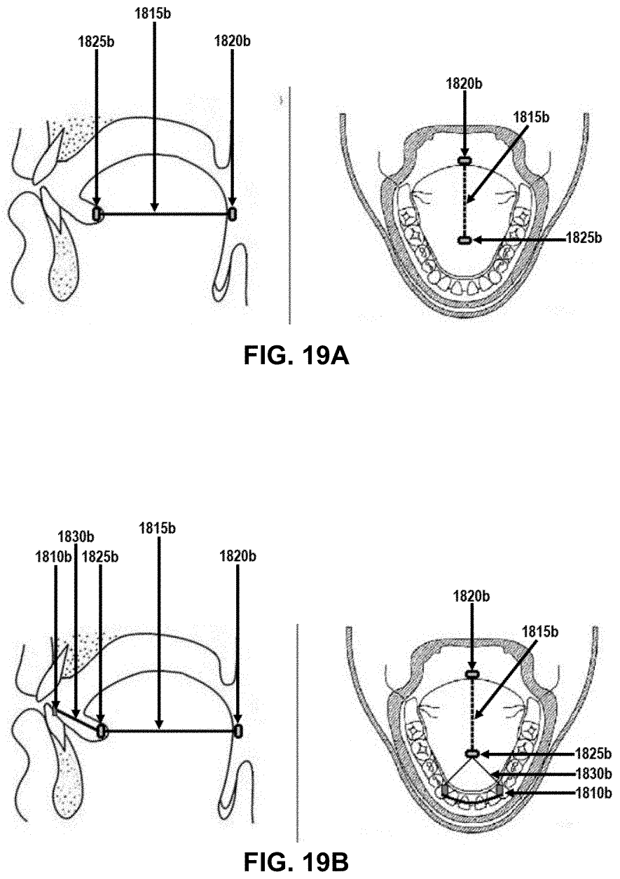

FIGS. 19A and 19B illustrate embodiments in which the multi-component device includes one retractor member 1820b, one anchor member 1825b, and either one elongate member or two elongate members 1815b extending between the retractor member 1820b and the anchor member 1825b. In FIG. 19A, the elongate member(s) 1815b may extend from the retractor member 1820b through the tissue of tongue 112 and connect to the anchor member 1825b. When the retractor member 1820b is comprised of stud head 600k, an elongate member 1815b extends from the central body of the stud head 600k of the retractor member 1820b. The end of the elongate member 1815b may be pulled through the anchor member 1825b to adjust the tension before securement of the end of the elongate member 1815b to the anchor member 1825b. For example, when the anchor member 1825b is comprised of stud head 600l, the elongate member 1815b may be slipped through the hole of a flange F of stud head 600l of the anchor member 1825b, adjusted in length, and then knotted off to lock the flange F of the anchor member 1825b against the tongue 112. As another example, when the anchor member 1825b is comprised of stud head 600m, the end of the elongate member 1815b may be slipped through the hole of a flange F of stud head 600m of the anchor member 1825b, adjusted in length, and then forcibly pulled through the hole of an O-ring OR of stud head 600m that locks the flange F of the anchor member 1825b against the tongue 112. When the retractor member 1820b is comprised of stud head 600j, first and second elongate members 1815b extends from the central body of the stud head 600j of the retractor member 1820b. The ends of the first and second the elongate members 1815b may be pulled through the anchor member 1825b to adjust the tension before securement of the ends of the first and second elongate members 1815b to the anchor member 1825b. As another example, when the anchor member 1825b is comprised of stud head 600n, the ends of the first and second elongate members 1815b may be slipped through the first and second holes of a flange F of stud head 600n of the anchor member 1825b, respectively, adjusted in length, and then knotted off to lock the flange F of the anchor member 1825b against the tongue 112. As shown in FIGS. 19A and 19B, the elongate member 1815b may be inserted through the tongue 112 or tongue base 113 at a midline position of the tongue 112 or tongue base 113. For example, the elongate member 1815b may be inserted at an anterior, midline location of the tongue 112, avoiding nerves, arteries, and taste buds of the tongue 112. In FIG. 19B, the multi-component device may further include a dental securement 1810b and an anchor member connecting the dental securement 1810b to the anchor member 1825b. The dental securement 1810b may include one or more attachment points, allowing the connector 1830b to attach to the dental securement 1810b. In such embodiments, the anchor member 1825b may also include an attachment member, allowing the connector 1830b to attach to the anchor member 1825b.

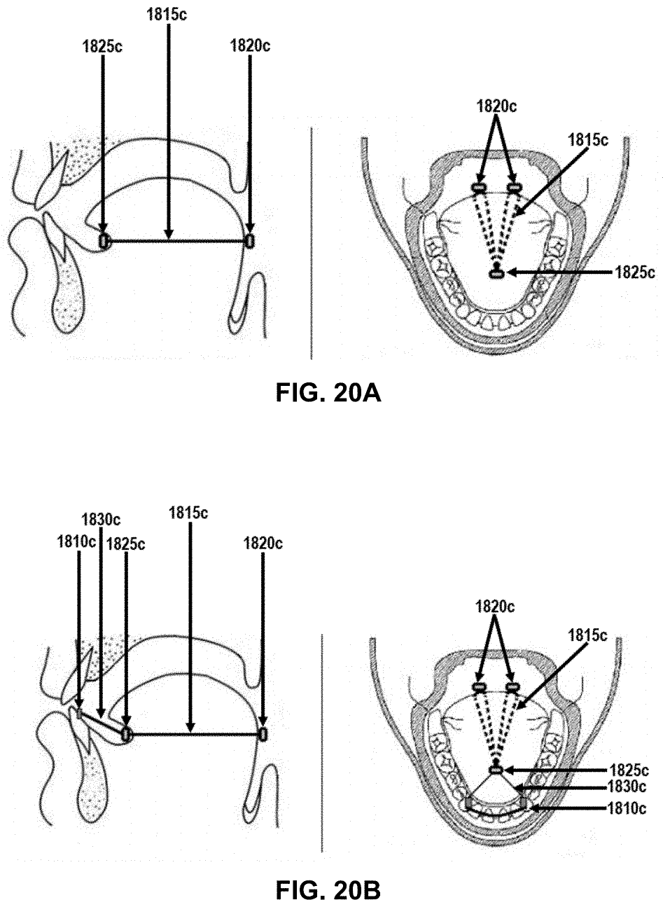

FIGS. 20A and 20B illustrate embodiments in which the multi-component device includes first and second retractor members 1820c, one anchor member 1825c, and first and second elongate members 1815c, each extending between the first and second retractor members 1820c and the anchor member 1825c. In FIG. 20A, the first elongate member 1815c may extend from the anchor member 1825c to the first retractor member 1820c through the tissue of tongue 112, exit the tissue of the tongue 112 in the region of the pharynx, connect to the first retractor member 1820c, and then extend from the first retractor member 1820c to the anchor member 1825c through the tissue of tongue 112. The second elongate member 1815c may extend from the anchor member 1825c to the second retractor member 1820c through the tissue of tongue 112, the tissue of the tongue 112, connect to the second retractor member 1820c, and then extend from the second exit retractor member 1820c to the anchor member 1825c through the tissue of tongue 112. The first and second elongate members 1815c may be connected, respectively, to the first and second retractor members 1820c by a loop. For example, when the first and second retractor members 1820c are each comprised of stud head 600h, the first and second elongate members 1815c may extend through the hole of a flange F and loop around the button B of each stud head 600h of the first and second retractor members 1820c, respectively. As another example, when the first and second retractor members 1820c are each comprised of stud head 600i, the first and second elongate members 1815c may loop through the holes of the flange F of each stud head 600i of the first and second retractor members 1820c, respectively. The ends of the first and second elongate members 1815c may be pulled through the anchor member 1825c to adjust the tension before securement of the ends of the first and second elongate members 1815c to the anchor member 1825c. For example, when the anchor member 1825c is comprised of stud head 600m, the ends of the first and second elongate members 1815c may be slipped through the hole of a flange F of stud head 600m of the anchor member 1825c, adjusted in length, and then forcibly pulled through the hole of an O-ring OR of stud head 600m of the anchor member 1825c that locks the flange F of the anchor member 1825c against the tongue 112. As another example, when the anchor member 1825c is comprised of stud head 600n, the ends of the first and second elongate members 1815c may be slipped through the first and second holes of a flange F of stud head 600n of the anchor member 1825c, respectively, adjusted in length, and knotted off to lock the flange F of the anchor member 1825c against the tongue 112. As shown in FIGS. 20A and 20B, the first and second elongate members 1815a may be inserted through the tongue 112 or tongue base 113 at a midline position of the tongue 112 or tongue base 113, and extend at an angle through the tissue of the tongue 112 or tongue base 113. For example, the first and second elongate members 1815c may be inserted at an anterior, midline location of the tongue 112, avoiding nerves, arteries, and taste buds of the tongue 112. In FIG. 20B, the multi-component device may further include a dental securement 1810c and connector 1830c connecting the dental securement 1810c to the anchor member 1825c. The dental securement 1810c may include one or more attachment points, allowing the connector 1830c to attach to the dental securement 1810c. In such embodiments, the anchor member 1825c may also include an attachment member, allowing the connector 1830c to attach to the anchor member 1825c.

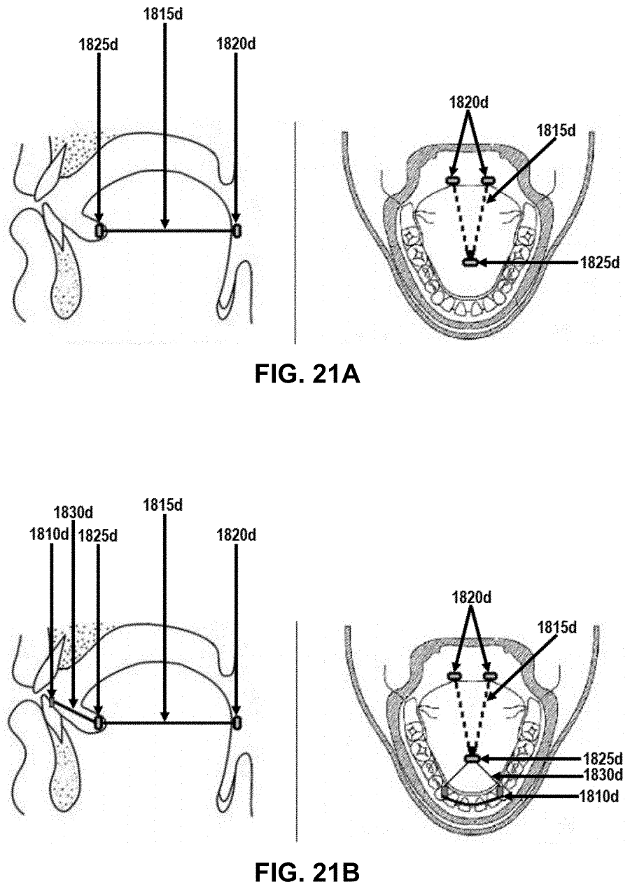

FIGS. 21A and 21B illustrate embodiments in which the multi-component device includes first and second retractor members 1820d, one anchor member 1825d, and either one elongate member 1815d or two elongate members 1815d extending between each retractor member 1820d and the anchor member 1825d. In FIG. 21A, the first elongate member(s) 1815d may extend from the first retractor member 1820d through the tissue of tongue 112 and connect to the anchor member 1825d. The second elongate member(s) 1815d may extend from the second retractor member 1820d through the tissue of tongue 112, and connect to the anchor member 1825d. When the first and second retractor members 1820d is each comprised of stud head 600k, first and second elongate members 1815d extend from the central body of the first and second stud heads 600k of the first and second retractor members 1820d, respectively. The ends of the first and second elongate members 1815d may be pulled through the anchor member 1825d to adjust the tension of the first and second elongate members 1815d before securement of the ends of the first and second elongate members 1815d to the anchor member 1825d. For example, when the anchor member 1825d is comprised of stud head 600l, the ends of the first and second elongate members 1815d may be slipped through the flange hole of stud head 600l of the anchor member 1825d, adjusted in length, and then knotted off to lock the flange F of the anchor member 1825d against the tongue 112. As another example, when the anchor member 1825d is comprised of stud head 600m, the ends of the first and second elongate members 1815d may be slipped through the hole of a flange F of stud head 600m of the anchor member 1825d, adjusted in length, and then forcibly pulled through the hole of an O-ring OR of stud head 600m of the anchor member 1825d that locks the flange F of the anchor member 1825d against the tongue 112. When the first and second retractor members 1820d is each comprised of stud head 600j, a first and second elongate members 1815d extends from the central body of each stud head 600j of the first and second retractor members 1820d, respectively. The ends of the elongate members 1815d may be pulled through the anchor member 1825d to adjust the tension before securement of the ends of the elongate members 1815d to the anchor member 1825d. As another example, when the anchor member 1825d is comprised of stud head 600n, the ends of the first and second elongate members 1815d may be slipped through the first and second holes of a flange F of stud head 600n of the anchor member 1825d, respectively, adjusted in length, and then knotted off to lock the flange F of the anchor member 1825d against the tongue 112. As shown in FIGS. 21A and 21B, the first and second elongate members 1815d may be inserted through the tongue 112 or tongue base 113 at a midline position of the tongue 112 or tongue base 113, and extend at an angle through the tissue of the tongue 112 or tongue base 113. For example, the first and second elongate members 1815d may be inserted at an anterior, midline location of the tongue 112, avoiding nerves, arteries, and taste buds of the tongue 112. In FIG. 21B, the multi-component device may further include a dental securement 1810d and connector 1830d connecting the dental securement 1810d to the anchor member 1825d. The dental securement 1810d may include one or more attachment points, allowing the connector 1830d to attach to the dental securement 1810d. In such embodiments, the anchor member 1825d may also include an attachment member, allowing the connector 1830d to attach to the anchor member 1825d.

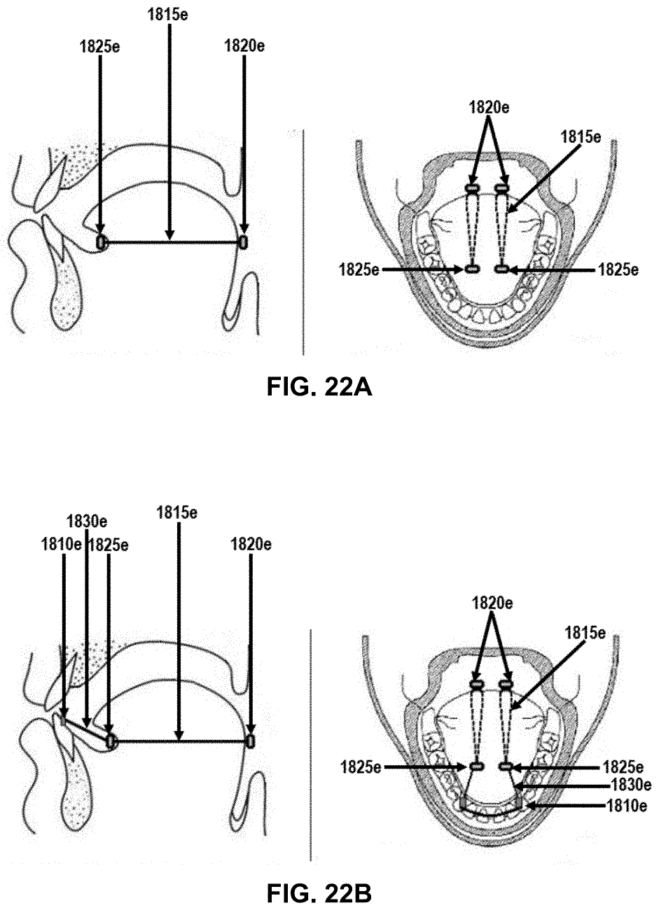

FIGS. 22A and 22B illustrate embodiments in which the multi-component device includes first and second retractor members 1820e, first and second anchor members 1825e, and first and second elongate members 1815e, each extending between the first and second retractor members 1820e and the first and second anchor members 1825e, respectively. In FIG. 22A, the first elongate member 1815e may extend from the first anchor member 1825e to the first retractor member 1820e through the tissue of tongue 112, exit the tissue of the tongue 112, connect to the first retractor member 1820e, and then extend from the first retractor member 1820e to the first anchor member 1825e through the tissue of tongue 112. The second elongate member 1815e may extend from the second anchor member 1825e to the second retractor member 1820e through the tissue of tongue 112, exit the tissue of the tongue 112, connect to the second retractor member 1820e, and then extend from the second retractor member 1820e to the second anchor member 1825e through the tissue of tongue 112. At one end, the first and second elongate members 1815e may be connected, respectively, to the first and second retractor members 1820e by a loop. For example, when the first and second retractor members 1820e are each comprised of stud head 600h, the first and second elongate members 1815e may extend through the hole of a flange F and loop around the button B of each stud head 600h of the first and second retractor members 1820e, respectively. As another example, when the first and second retractor members 1820e are each comprised of stud head 600i, the first and second elongate members 1815e loop through the holes of the flange F of each stud head 600i of the first and second retractor members 1820e, respectively. The ends of the first and second elongate members 1815e may be pulled through the first and second anchor members 1825e, respectively, to adjust the tension of the first and second elongate members 1815e before securement of the ends of the first and second elongate members 1815e to the first and second anchor members 1825e, respectively. For example, when the first and second anchor members 1825e are each comprised of stud head 600m, the ends of the first and second elongate members 1815e may be slipped through the hole of a flange F of stud head 600m of the first and second anchor members 1825e, respectively, and then forcibly pulled through the hole of the O-ring OR of stud head 600m of the first and second anchor members 1825e, respectively, that then locks the flange F of the first and second anchor members 1825e against the tongue 112. As another example, when the first and second anchor members 1825e are each comprised of stud head 600n, the ends of the first and second elongate members 1815e may be slipped through the first and second holes of each flange F of each stud head 600n of the first and second anchor members 1825e, respectively, and the ends of the first and second elongate members 1815e are then knotted off to lock the flange F of the first and second anchor members 1825e against the tongue 112. As shown in FIGS. 22A and 22B, the first and second elongate members 1815e may be inserted through the tongue 112 or tongue base 113 at a location offset from a midline of the tongue 112. In FIG. 22B, the multi-component device may further include a dental securement 1810e and first and second connectors 1830e connecting the dental securement 1810e to the first and second anchor members 1825e. The dental securement 1810e may include one or more attachment points, allowing the first and second connectors 1830e to attach to the dental securement 1810e. In such embodiments, the first and second anchor members 1825e may also include an attachment member, allowing the connector 1830e to attach to the anchor member 1825e.

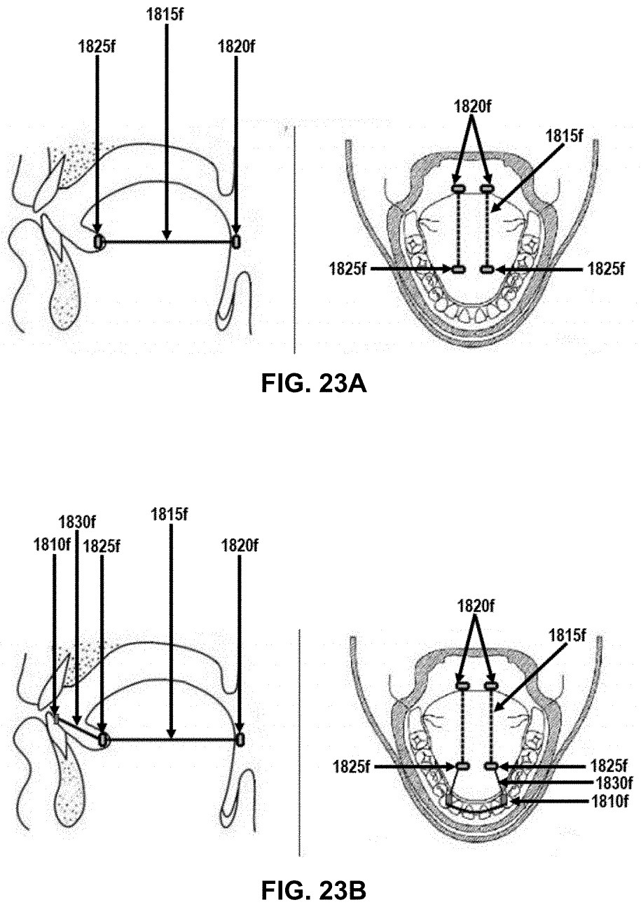

FIGS. 23A and 23B illustrate embodiments in which the multi-component device includes first and second retractor members 1820f, first and second anchor members 1825f, and either one elongate member 1815f, or two elongate members 1815f, extending between the first and second retractor members 1820f and the first and second anchor members 1825f, respectively. In FIG. 23A, the first elongate member(s) 1815f may extend from the first retractor member 1820f through the tissue of tongue 112, and connect the first anchor member 1825f. The second elongate member(s) 1815f may extend from the second retractor member 1820f through the tissue of tongue 112, and connect to the second anchor member 1825f. When the first and second retractor member 1820f is each comprised of stud head 600k, a first and second elongate member 1815f extends from the central body of each stud head 600k of the first and second retractor members 1820f, respectively. The ends of the first and second elongate members 1815f may be pulled through the first and second anchor members 1825f, respectively, to adjust the tension of the first and second elongate members 1815f before securement of the ends of the first and second elongate members 1815f to the first and second anchor members 1825f, respectively. For example, when the first and second anchor members 1825f are each comprised of stud head 600l, the ends of the first and second elongate members 1815f may be slipped through the hole of the flange F of stud head 600l of the first and second anchor members 1825f, respectively, adjusted in length, and then knotted off to lock the flange F of the first and second anchor members 1825f against the tongue 112. As another example, when the first and second anchor members 1825f are each comprised of stud head 600m, the ends of the first and second elongate members 1815f may be slipped through the hole of the flange F of stud head 600m of the first and second anchor members 1825f, respectively, adjusted in length, and then forcibly pulled through the hole an O-ring OR of stud head 600m of the first and second anchor members 1825f, respectively, that locks the flanges F of the first and second anchor members 1825f against the tongue 112. When the first and second retractor members 1820f is each comprised of stud head 600j, a first and second elongate members 1815f extends from the central body of each stud head 600j of the first and second retractor members 1820f, respectively. The ends of the first and second elongate members 1815f of the first and second retractor members 1820f may be pulled through the first and second anchor members 1825f, respectively, to adjust the tension of the first and second elongate members 1815f before securement of the ends of the first and second elongate members 1815f to the first and second anchor members 1825f, respectively. For example, when the first and second anchor members 1825f are each comprised of stud head 600m, the ends of the first and second elongate members 1815f may be slipped through the hole of a flange F of stud head 600m of the first and second anchor members 1825f, respectively, and then forcibly pulled through the hole of the O-ring OR of stud head 600m of the first and second anchor members 1825f, respectively, that then locks the flange F of the first and second anchor members 1825f against the tongue 112. As another example, when the first and second anchor members 1825f are each comprised of stud head 600n, the ends of the first and second elongate members 1815f may be slipped through the first and second holes of a flange F of stud head 600n of the first and second anchor members 1825f, respectively, and the ends of the first and second elongate members 1815f are then knotted off, respectively, to lock the flange F of the first and second anchor members 1825f against the tongue 112. As shown in FIGS. 23A and 23B, the first and second elongate members 1815f may be inserted through the tongue 112 or tongue base 113 at a location offset from a midline of the tongue 112. In FIG. 23B, the multi-component device may further include a dental securement 1810f and first and second connectors 1830f connecting the dental securement 1810f to the first and second anchor members 1825f. The dental securement 1810f may include one or more attachment points, allowing the first and second connectors 1830f to attach to the dental securement 1810f. In such embodiments, the first and second anchor members 1825f may also include an attachment member, allowing the connector 1830f to attach to the anchor member 1825f.

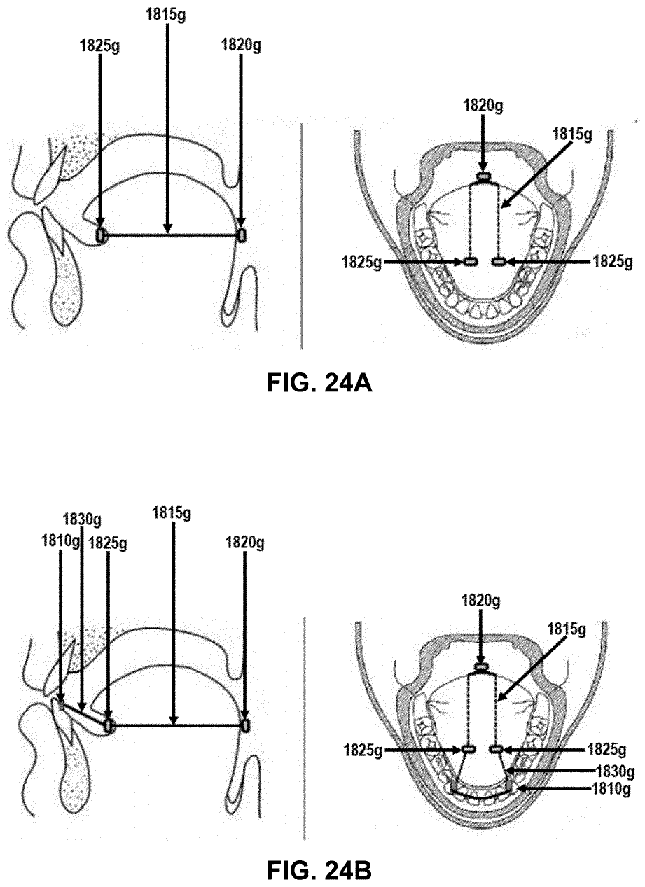

FIGS. 24A and 24B illustrate embodiments in which the multi-component device includes one retractor member 1820g, first and second anchor members 1825g, and either one elongate member 1815g extending between the first and second anchor members 1825g and the retractor member 1820g or, a first and second elongate members 1815g extending between the first and second anchor members 1825g and the retractor member 1820g, respectively. In FIG. 24A, the first and second elongate members 1815g may extend from the retractor member 1820g through the tissue of tongue 112 to connect to the first and second anchor members 1825g, respectively. Alternatively, the elongate member 1815g may extend from the first anchor member 1825g to the retractor member 1820g through the tissue of tongue 112, exit the tissue of the tongue 112, connect to the retractor member 1820g, and then extend from the retractor member 1820g to the second anchor member 1825g through the tissue of tongue 112. The elongate member 1815g may be connected to the retractor member 1820g by a loop. For example, when the retractor member 1820g is comprised of stud head 600h, the elongate member 1815g may extend through the hole of a flange F and loop around a button B of stud head 600h of the retractor member 1820g. As another example, when the retractor member 1820g is comprised of stud head 600i, the elongate member 1815g may be looped through the holes of the flange F of stud head 600i of the retractor member 1820g. The first and second ends of elongate member 1815g may be pulled through the first and second anchor members 1825g, respectively, to adjust the tension of the elongate member 1815g before securement of the first and second ends of elongate member 1815g to the first and second anchor members 1825g, respectively. For example, when the first and second anchor members 1825g are each comprised of stud head 600l, the first and second ends of elongate member 1815g may be slipped through the hole of the flange F of stud head 600l of the first and second anchor members 1825g, respectively, adjusted in length, and the first and second ends of elongate members 1815g knotted off to lock the flange F of the first and second anchor members 1825g against the tongue 112. As another example, when the first and second anchor members 1825g are each comprised of stud head 600m, the first and second ends of elongate members 1815g may be slipped through the hole of the flange F of stud head 600m of the first and second anchor members 1825g, respectively, adjusted in length, and then forcibly pulled through the hole an O-ring of stud head 600m of the first and second anchor members 1825g, respectively, that locks the flanges F of the first and second anchor members 1825g against the tongue 112. When the retractor member 1820g is comprised of stud head 600j, first and second elongate members 1815g extends from the central body of the stud head 600j of the retractor member 1820g. The ends of the first and second elongate members 1815g of the retractor member 1820g may be pulled through the first and second anchor members 1825g, respectively, to adjust the tension of the first and second elongate members 1815g before securement of the ends of the first and second elongate members 1815g to the first and second anchor members 1825g, respectively. For example, when the first and second anchor members 1825g are each comprised of stud head 600l, the ends of the first and second elongate members 1815g may be slipped through the hole of the flange F of stud head 600l of the first and second anchor members 1825g, respectively, adjusted in length, and then knotted off to lock the flange F of the first and second anchor members 1825g against the tongue 112. As another example, when the first and second anchor members 1825g are each comprised of stud head 600m, the ends of the first and second elongate members 1815g may be slipped through the hole of a flange F of stud head 600m of the first and second anchor members, respectively, and then forcibly pulled through the hole of the O-ring OR of stud head 600m of the first and second anchor members, respectively, that then locks the flange F of the first and second anchor members against the tongue 112. As shown in FIGS. 24A and 24B, the elongate member(s) 1815g may be inserted through the tongue 112 or tongue base 113 at a location offset from a midline of the tongue 112. In FIG. 24B, the multi-component device may further include a dental securement 1810g and first and second connectors 1830g connecting the dental securement 1810g to the first and second anchor members 1825g. The dental securement 1810g may include one or more attachment points, allowing the first and second connectors 1830g to attach to the dental securement 1810g. In such embodiments, the first and second anchor members 1825g may also include an attachment member, allowing the connectors 1830g to attach to the anchor members 1825g.

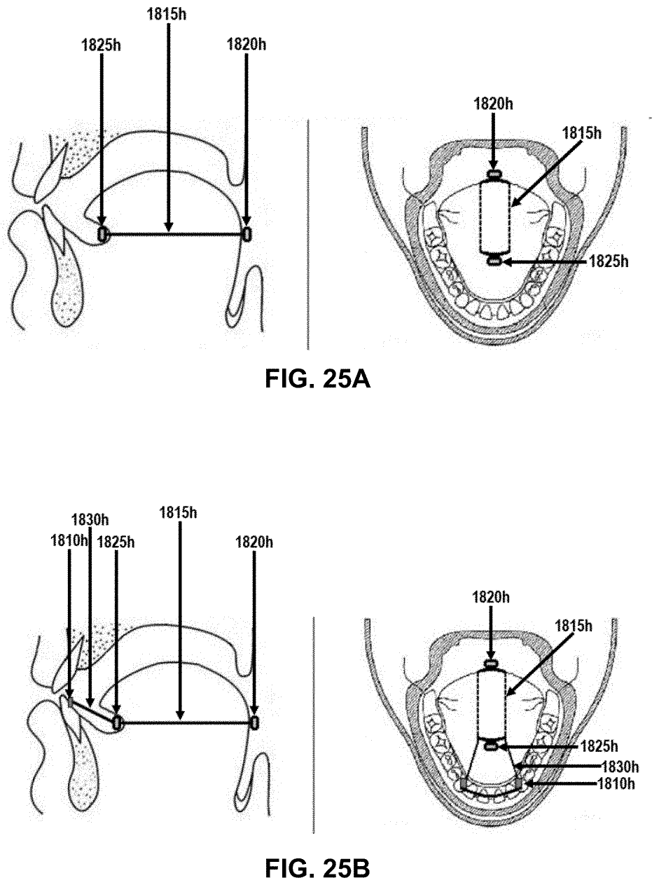

FIGS. 25A and 25B illustrate embodiments in which the multi-component device includes one retractor member 1820h, one anchor member 1825h, and either one elongate member 1815h extending between the anchor member 1825h and the retractor member 1820h or, a first and second elongate members 1815h extending between the anchor member 1825h and the retractor member 1820h. In FIG. 25A, the first and second elongate members 1815h may extend from the retractor member 1820h through the tissue of tongue 112 to connect to the anchor member 1825h. Alternatively, the elongate member 1815h may extend from the anchor member 1825h to the retractor member 1820h through the tissue of tongue 112, exit the tissue of the tongue 112, connect to the retractor member 1820h, and then extend from the retractor member 1820h back to the anchor member 1825h through the tissue of tongue 112. The elongate member 1815h may be connected to the retractor member 1820h by a loop. For example, when the retractor member 1820h is comprised of stud head 600h, the elongate member 1815h may extend through the hole of a flange F and loop around a button B of stud head 600h of the retractor member 1820h. As another example, when the retractor member 1820h is comprised of stud head 600i, the elongate member 1815h may be looped through the holes of the flange F of stud head 600i of the retractor member 1820h. The first and second ends of the elongate member 1815h may be pulled through the anchor member 1825h to adjust the tension of the elongate member 1815h before securement of the ends of the elongate member 1815h to the anchor member 1825h. For example, when the anchor member 1825h is comprised of stud head 600l, the first and second ends of the elongate member 1815h may be slipped through the flange hole of stud head 600l of the anchor member 1825h, adjusted in length, and then knotted off to lock the flange F against the tongue 112. As another example, when the anchor member 1825h is comprised of stud head 600m, the first and second ends of the elongate member 1815h may be slipped through the hole of a flange F of stud head 600m of the anchor member 1825h, adjusted in length, and then forcibly pulled through the hole of an O-ring OR of stud head 600m of the anchor member 1825h that locks the flange F against the tongue 112. When the retractor member 1820h is comprised of stud head 600j, first and second elongate members 1815h extends from the central body of the stud head 600j of the retractor member 1820h. The ends of the first and second elongate members 1815h may be pulled through the anchor member 1825h to adjust the tension before securement of the ends of the first and second elongate members 1815h to the anchor member 1825h. For example, when the anchor member 1825h is comprised of stud head 600m, the ends of the first and second elongate members 1815h may be slipped through the hole of a flange F of stud head 600m of the anchor member 1825h, adjusted in length, and then forcibly pulled through the hole of an O-ring OR of stud head 600m of the anchor member 1825h that locks the flange F of the anchor member 1825h against the tongue 112. As another example, when the anchor member 1825h is comprised of stud head 600n, the ends of the first and second elongate members 1815h may be slipped through the first and second holes of a flange F of stud head 600n of the anchor member 1825h, respectively, adjusted in length, and then knotted off to lock the flange F of the anchor member 1825h against the tongue 112. As shown in FIGS. 25A and 25B, the elongate member(s) 1815h may be inserted through the tongue 112 or tongue base 113 at a location offset from a midline of the tongue 112. In FIG. 25B, the multi-component device may further include a dental securement 1810h and first and second connectors 1830h connecting the dental securement 1810h to the first and second anchor members 1825h. The dental securement 1810h may include one or more attachment points, allowing the first and second connectors 1830h to attach to the dental securement 1810h. In such embodiments, the anchor member 1825h may also include an attachment member, allowing the connectors 1830h to attach to the anchor member 1825h.

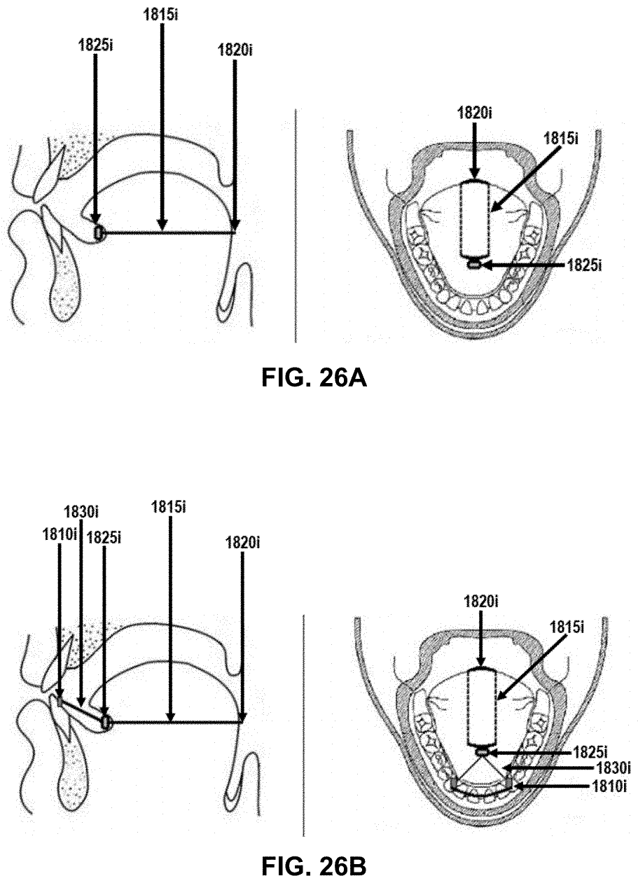

FIGS. 26A and 26B illustrate embodiments in which the multi-component device includes one anchor member 1825i and one elongate member 1815i. In FIG. 26A, the elongate member 1815i may extend from the anchor member 1825i through the tissue of tongue 112, exit the tissue of the tongue 112 and then extend back to the anchor member 1825i through the tissue of the tongue 112. The first and second ends of the elongate member 1815i may be pulled through the anchor member 1825i to adjust the tension of the elongate member 1815i before securement of the first and second ends of the elongate member 1815i to the anchor member 1825i. For example, when the anchor member 1825i is comprised of stud head 600l, first and second ends of the elongate member 1815i may be slipped through the flange hole of stud head 600l of the anchor member 1825i, adjusted in length, and then knotted off to lock the flange F of the anchor member 1825i against the tongue 112. As another example, when the anchor member 1825i is comprised of stud head 600m, the first and second ends of the elongate member 1815i may be slipped through the hole of a flange F of stud head 600m of the anchor member 1825i, adjusted in length, and then forcibly pulled through the hole of an O-ring OR of stud head 600m of the anchor member 1825i that locks the flange F of the anchor member 1825i against the tongue 112. As a further example, when the anchor member 1825i is comprised of stud head 600n, the first and second ends of the elongate member 1815i may be slipped through the first and second holes of a flange F of stud head 600n of the anchor member 1825i, respectively, adjusted in length, and then knotted off to lock the flange F of the anchor member 1825i against the tongue 112. As shown in FIGS. 26A and 26B, the elongate member 1815i may be inserted through the tongue 112 or tongue base 113 at a location offset from a midline of the tongue 112. In FIG. 26B, the multi-component device may further include a dental securement 1810i and first and second connectors 1830i connecting the dental securement 1810i to the first and second anchor members 1825i. The dental securement 1810i may include one or more attachment points, allowing the first and second connectors 1830i to attach to the dental securement 1810i. In such embodiments, the anchor member 1825i may also include an attachment member, allowing the connectors 1830i to attach to the anchor member 1825i.

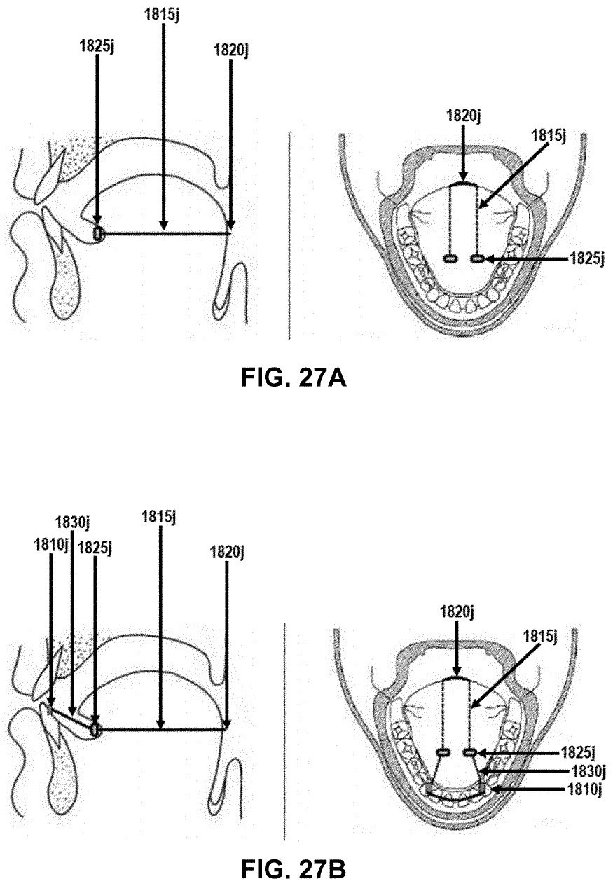

FIGS. 27A and 27B illustrate embodiments in which the multi-component device includes first and second anchor members 1825j and one elongate member 1815j. In FIG. 27A, the elongate member 1815j may extend from the first anchor member 1825j through the tissue of tongue 112, exit the tissue of the tongue 112, re-enter through the tissue of the tongue 112, and extend to the second anchor member 1825j. The first and second ends of the elongate member 1815j may be pulled through the first and second anchor members 1825j, respectively, to adjust the tension of the elongate member 1815j before securement of the first and second ends of the elongate member 1815j to the first and second anchor members 1825j, respectively. For example, when the first and second anchor members 1825j are comprised of stud heads 600l, the first and second ends of the elongate member 1815j may be slipped through the flange hole of the first and second stud heads 600l of the first and second anchor members 1825j, respectively, adjusted in length, and then knotted off to lock the flange F of the stud head 600l of the first and second anchor members 1825j against the tongue 112. As another example, when the first and second anchor members 1825j are each comprised of stud head 600m, the first and second ends of the elongate member 1815j may be slipped through the hole of the flange F of stud head 600m of the first and second anchor members 1825j, respectively, adjusted in length, and then forcibly pulled through the hole an O-ring OR of stud head 600m of the first and second anchor members 1825j, respectively, that locks the flanges F of the first and second anchor members 1825j against the tongue 112. As shown in FIGS. 27A and 27B, the elongate member 1815j may be inserted through the tongue 112 or tongue base 113 at a location offset from a midline of the tongue 112. In FIG. 27B, the multi-component device may further include a dental securement 1810j and first and second connectors 1830j connecting the dental securement 1810j to the first and second anchor members 1825j. The dental securement 1810j may include one or more attachment points, allowing the first and second connectors 1830j to attach to the dental securement 1810j. In such embodiments, the first and second anchor members 1825j may also include an attachment member, allowing the connectors 1830j to attach to the anchor members 1825j.

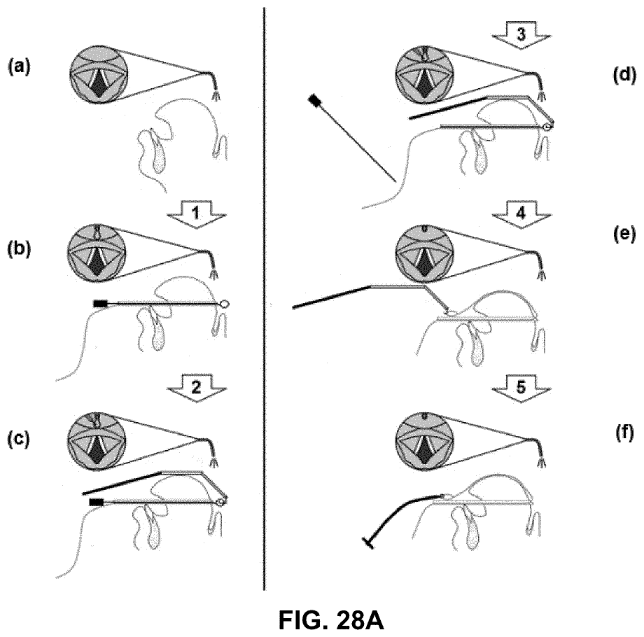

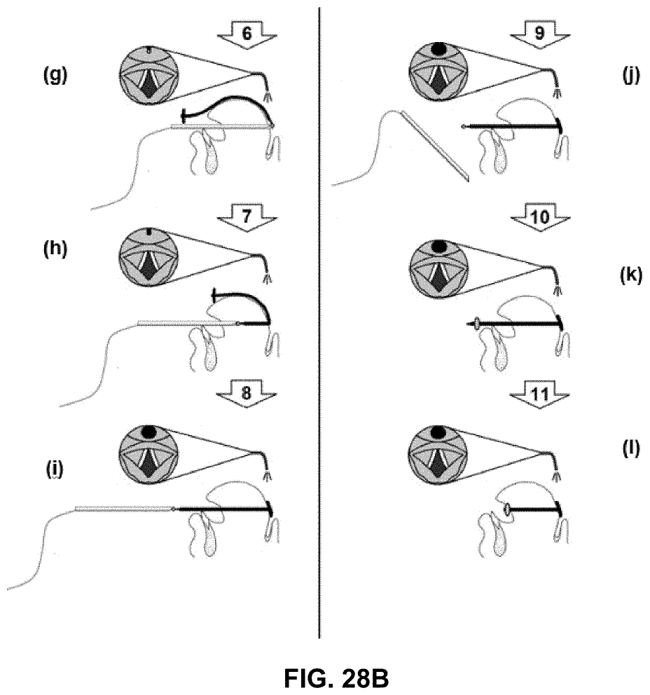

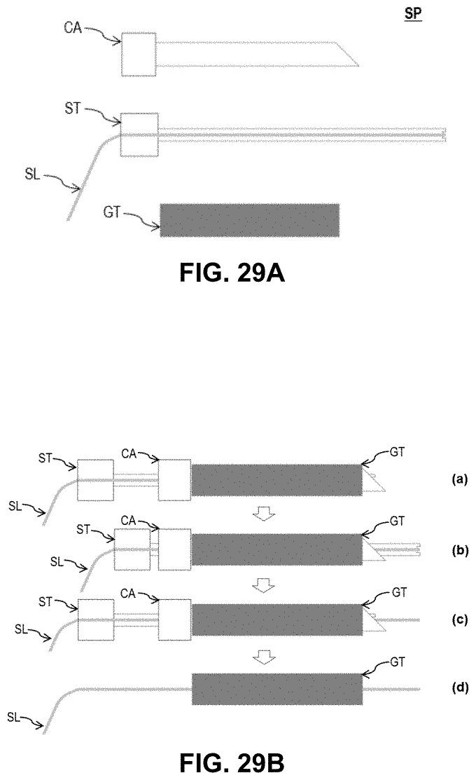

FIGS. 28A-28B and 29A-29B illustrate the method of placement of components of a multi-component device used in glossopexy, consistent with certain exemplary embodiments. Specifically, each figure for FIGS. 28A-28B includes both a cross-sectional and endoscopic perspective of the tongue and surrounding structures to illustrate placement of components of a multi-component device used in glossopexy, consistent with certain exemplary embodiments. For example, FIGS. 28A-28B illustrate the embodiment of FIG. 19A, including one retractor member 1820b, one anchor member 1825b, and one elongate member 1815b extending between the retractor member 1820b and the anchor member 1825b. As illustrated in FIGS. 28A-28B, the retractor member 1820b, the anchor member 1825b, and the elongate member 1815b may operate together to maintain a position of, or bring forward, the tongue 112 in the oral cavity 111, thereby maintaining an open passage through the oropharynx 115.

FIGS. 29A-29B show views of the insertion tool used in connection with the placement of the components of the multi-component device, consistent with certain exemplary embodiments. As discussed more fully below, the insertion tool of FIGS. 29A-29B may be a suture passer SP comprising a guidance tube GT coaxially jacketing a needle canula CA that centrally contains a blunt-ended stylet ST.

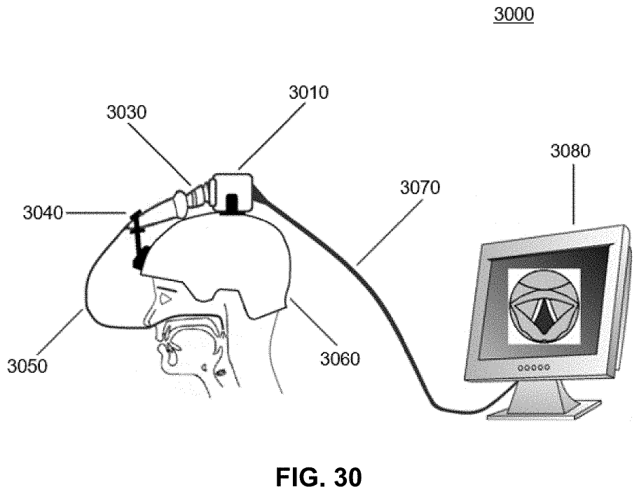

FIG. 30 illustrates an endoscopic helmet camera system for use with the placement of the components of the multi-component device, consistent with certain exemplary embodiments. As shown in FIG. 30, the endoscopic helmet camera system 3000 may comprise a helmet 3060, a flexible fiberoptic endoscope 3050, an endoscope bracket 3040, an endoscope eyepiece 3030, an endoscope camera head 3010, a video cable 3070, and a display device 3080. The helmet 3060 may support the flexible fiberoptic endoscope 3050, the endoscope bracket 3040, the endoscope eyepiece 3030, and the endoscope camera head 3010. The flexible fiberoptic endoscope 3050 may be inserted in a nostril of a patient, and may extend through the nasal cavity 110 into the nasopharynx 114 and to the oropharynx 115. The endoscopic camera head 3010 may be connected to the display device via a video cable 3070, and may allow for images that are captured by the flexible fiberoptic endoscope 3050 to be displayed on the display device 3080. For example, the flexible fiberoptic endoscope 3050 may capture images in the region of the oropharynx 115, and may transmit the captured images to the display device 3080 via the video cable 3070 using known communication standards and protocols.

Although not illustrated, the display device 3080 may be connected to a controller. The controller can include one or more of the following components: at least one central processing unit (CPU) configured to execute computer program instructions (e.g., software) to perform various processes and methods, a graphics processor (GPU), random access memory (RAM) and read only memory (ROM) configured to access and store data and information and computer program instructions, input/output (I/O) devices configured to provide input and/or output to the controller (e.g., keyboard, mouse, display, speakers, printers, modems, network cards, etc.), and storage media or other suitable type of memory (e.g., such as, for example, RAM, ROM, programmable read-only memory (PROM), erasable programmable read-only memory (EPROM), electrically erasable programmable read-only memory (EEPROM), magnetic disks, optical disks, floppy disks, hard disks, removable cartridges, flash drives, any type of tangible and non-transitory storage medium) where data and/or instructions can be stored. In addition, the controller can include antennas, network interfaces that provide wireless and/or wire line digital and/or analog interface to one or more networks over one or more network connections (not shown), a power source that provides an appropriate alternating current (AC) or direct current (DC) to power one or more components of the controller, and a bus that allows communication among the various disclosed components of the controller. As is understood, “software” refers to prescribed rules to operate a computer, such as code or script.

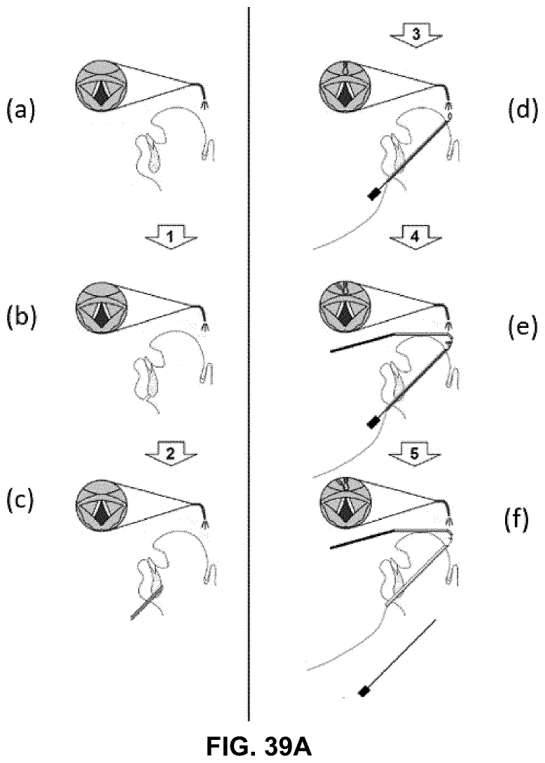

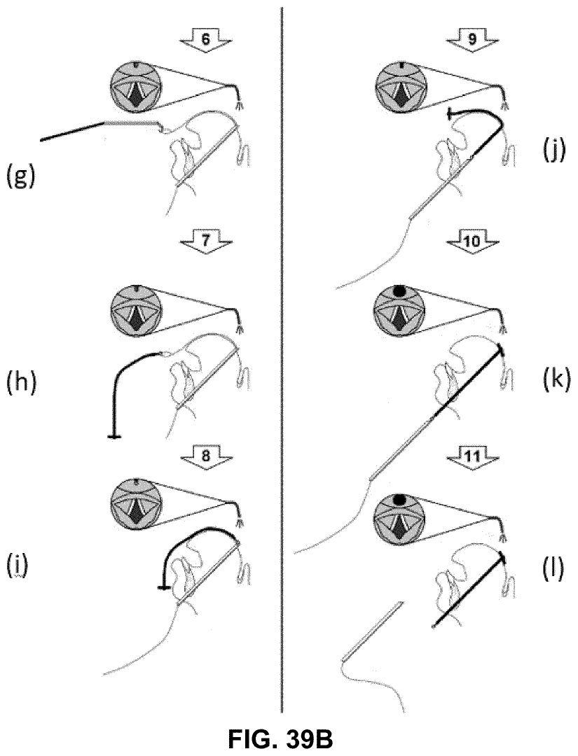

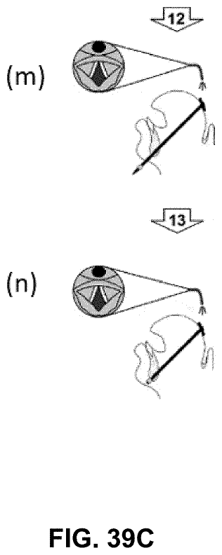

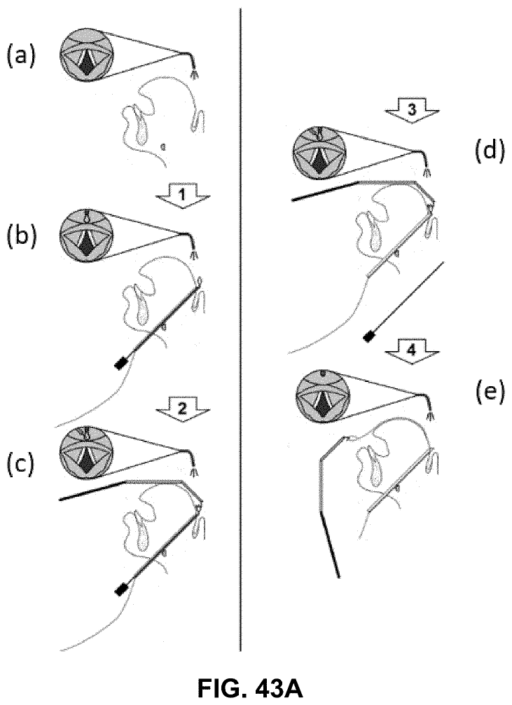

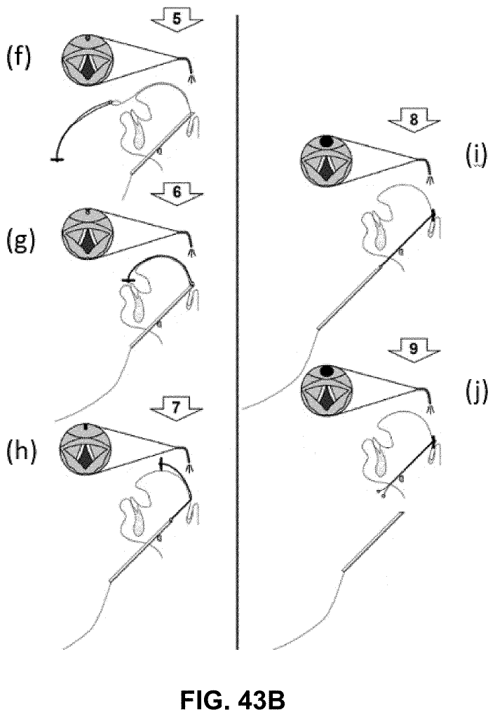

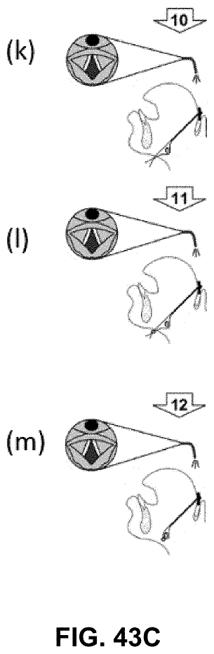

Referring to FIG. 28A-B, and FIG. 30, FIG. 39A-C, FIG. 43A-C, the purpose of the endoscopic helmet camera system 3000 is to allow the continuous “hands-free” visualization of the tongue base 113. The system thereby enables the surgeon to use one hand to retract the tongue while using the other hand to perform surgical maneuvers. These maneuvers may include the advancement of the suture passer SP safely through the tongue base 113 into the oropharynx 115 and the retrieval of the loop of suture SL over the top surface of the tongue 112 and external to the oral cavity 111 using a hooking tool HT. The transnasal positioning of the flexible fiberoptic endoscope 3050 also avoids the possibility of interference that would occur if the flexible fiberoptic endoscope 3050 was positioned transorally.

Referring to (a) of FIG. 28A, FIGS. 29A-29B, and FIG. 30, the flexible fiberoptic endoscope may be positioned within the oropharynx 115 of a patient, and in (b) an insertion tool may be used to pierce the tongue 112, making a hole in the tongue 112. In some examples, the insertion tool may be a suture passer SP inserted through the tissue of tongue 112. The suture passer SP may comprise a guidance tube GT coaxially jacketing a needle canula CA that centrally contains a blunt-ended stylet ST with a loop of suture SL seated inside an open notch located at the tip of the stylet ST (see also FIG. 29A and (a) of FIG. 29B). The retractable stylet ST may be advanced beyond the tip of the canula CA to release a loop of suture SL beyond the tip of the stylet ST into the oropharynx 115 (see also (b) of FIG. 29B). In (c) of FIG. 28A, a hooking tool HT may be extended over the top of the tongue 112 to engage and retain the loop of suture SL that extends beyond the tip of the suture passer SP. In (d) of FIG. 28A, the canula CA and stylet ST may be withdrawn in unison and removed while the guidance tube GT and loop of suture SL remain in position within the tissue of tongue 112 (see also (c) and (d) of FIG. 29B). The tip of the guidance tube GT may be located at the surface of the tongue 112 in the oropharynx 115 and, in (e) of FIG. 28A, the hooking tool HT may draw the loop of suture SL over the top surface of the tongue 112 and external to the oral cavity 111. Once the loop is outside of the mouth, as shown in (f) of FIG. 28A, the loop of suture SL may be connected to an end of the shaft S of the oral stud, and the oral stud may be drawn over the top surface of the tongue 112 to tightly contact the tip of the guidance tube GT in the oropharynx 115, as shown in (g) of FIG. 28B. The oral stud and guidance tube GT may be drawn forward in unison through the tissue of tongue 112 and outside of the mouth, as shown in (h) of FIG. 28B, such that the retractor member of the oral stud is pulled against the surface of the tongue 112, as shown in (i) of FIG. 28B. Then the loop of suture SL and guidance tube GT may be detached from the end of the shaft S, as shown in (j) of FIG. 28B, and an anchor member may be attached to the shaft, as shown in (k) of FIG. 28B. Finally, as shown in (l) of FIG. 28B, the shaft S may be adjusted in length to draw the retaining member and the anchor member against the external surface of the tongue 112. At each of steps (a) through (l) of FIGS. 28A-28B, the endoscopic helmet camera system of FIG. 30 may display images captured from the oropharynx 115 onto display device 3080.

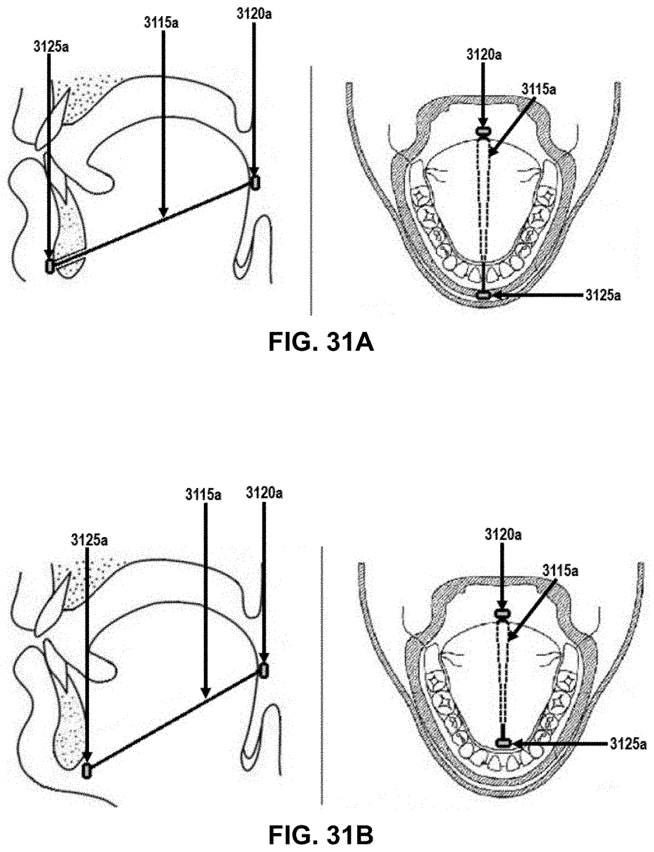

FIGS. 31A-31B, 32A-32B, 33A-33B, 34A-34B, 35A-35B, 36A-36B, 37A-37B, and 38A-38B are front and side views of a human head to illustrate placement of components of a multi-component device used in suspension glossomandibulopexy, consistent with certain exemplary embodiments. In the embodiments illustrated by FIGS. 31A-31B, 32A-32B, 33A-33B, 34A-34B, 35A-35B, 36A-36B, 37A-37B, and 38A-38B, the multi-component device may include at least one retractor member 3120, at least one anchor member 3125, and at least one elongate member 3115 extending between the at least one retractor member 3120 and the at least one anchor member 3125. The multi-component device illustrated by FIGS. 31A-31B, 32A-32B, 33A-33B, 34A-34B, 35A-35B, 36A-36B, 37A-37B, and 38A-38B may be used to alter the position of the tongue 112 and, in particular, move the tongue 112 anteriorly away from the oropharynx 115. For example, the tongue 112 may be shifted slightly forward in the oral cavity 111.

The at least one elongate member 3115 may be inserted into and extend through a patient's oral tissue, such as, for example, a mandible 118, a uvula 104, a tongue 112 or tongue base 113, or lateral pharyngeal walls 117. The at least one elongate member 3115 may be made of a biocompatible material, such as, for example, metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof. The at least one elongate member 3115 may include a shape memory material (SMM), such that the at least one elongate member 3115 is able to maintain and/or recover its original shape from a significant and seemingly plastic deformation when a particular stimulus is applied (e.g., superelasticity, visco-elasticity). For example, in each of the embodiments of FIGS. 31A-31B, 32A-32B, 33A-33B, 34A-34B, 35A-35B, 36A-36B, 37A-37B, and 38A-38B, the at least one elongate member 3115 may be formed of a material having superelasticity, such that the force of the at least one elongate member 3115 returning to its original shape causes a gentle, continuous pressure to be applied to the at least one retractor member 3120 and/or the at least one anchor member 3125 to which the at least one elongate member 3115 is connected. Each of the at least one retractor members 3120 and the at least one anchor members 3125 may be, for example, the stud heads 600 of FIGS. 6H-6N. In example embodiments, one or more of the at least one retractor member 3120 and the at least one anchor members 3125 may be provided in the region of the pharynx, a region of the tongue, a region of the mandible bone, or a region of the hyoid bone, as discussed below.

FIG. 31A illustrates an embodiment in which the multi-component device includes one retractor member 3120a, one anchor member 3125a, and one elongate member 3115a extending between the retractor member 3120a and the anchor member 3125a. In FIG. 31A, an anchor hole may be created. For example, an anchor hole may be drilled through the mandible 118 and the elongate member 3115a may be positioned to extend through the anchor hole. For example, the elongate member 3115a may extend from the anchor member 3125a provided at an anterior wall of the mandible 118 to the retractor member 3120a through the mandible 118 and the tissue of tongue 112, exit the tissue of the tongue 112 at the base of the tongue 112, connect to the retractor member 3120a, and then extend from the retractor member 3120a to the anchor member 3125a through the tissue of tongue 112 and the mandible 118. The anchor member 3125a and first and second ends of the elongate member 3115a may be located at the entrance of the anchor hole and external to the mandible 118. The elongate member 3115a may be connected to the retractor member 3120a by a loop. For example, when the retractor member 3120a is comprised of stud head 600h, the elongate member 3115a may extend through the hole of a flange F and loop around a button B of stud head 600h of the retractor member 3120a. As another example, when the retractor member 3120a is comprised of stud head 600i, the elongate member 3115a may be looped through the holes of the flange F of stud head 600i of the retractor member 3120a. The first and second ends of the elongate member 3115a may be pulled through the anchor member 3125a to adjust the tension before securement of the first and second ends of the elongate member 3115a to the anchor member 3125a. For example, when the anchor member 3125a is comprised of stud head 600m, the first and second ends of the elongate member 3115a may be slipped through the hole of a flange F of stud head 600m of the anchor member 3125a, adjusted in length, and then forcibly pulled through the hole of an O-ring OR of stud head 600m of the anchor member 3125a that locks the flange F against the mandible 118. As another example, when the anchor member 3125a is comprised of stud head 600n, the first and second ends of the elongate member 3125a may be slipped through the first and second holes of a flange F of stud head 600n of the anchor member 3125a, respectively, and knotted off to lock the flange F against the mandible 118. As shown in FIG. 31A, the elongate member 3115a may be inserted through the mandible 118 and tongue 112 or tongue base 113 at a midline position of the mandible 118 and tongue 112 or tongue base 113, respectively.

FIG. 31B illustrates an embodiment in which the multi-component device includes one retractor member 3120a, one anchor member 3125a, and one elongate member 3115a extending between the retractor member 3120a and the anchor member 3125a. In FIG. 31B, the elongate member 3115a may extend from the anchor member 3125a through the tissue of tongue 112, exit the tissue of the tongue 112 at the base of the tongue 112, connect to the retractor member 3120a, and then extend from the retractor member 3120a to the anchor member 3125a through the tissue of tongue 112. The anchor member 3125a and first and second ends of the elongate member 3115a may be located at a position below the mandible 118 in the facial tissue. For example, the anchor member 3125a and the first and second ends of the elongate member 3115a may be located in a midline region subjacent to the mandible 118. Examples of the retractor member 3120a and the anchor member 3125a of FIG. 31B, and their respective connections with elongate member 3115a, may be the same as those discussed above in connection with FIG. 31A.

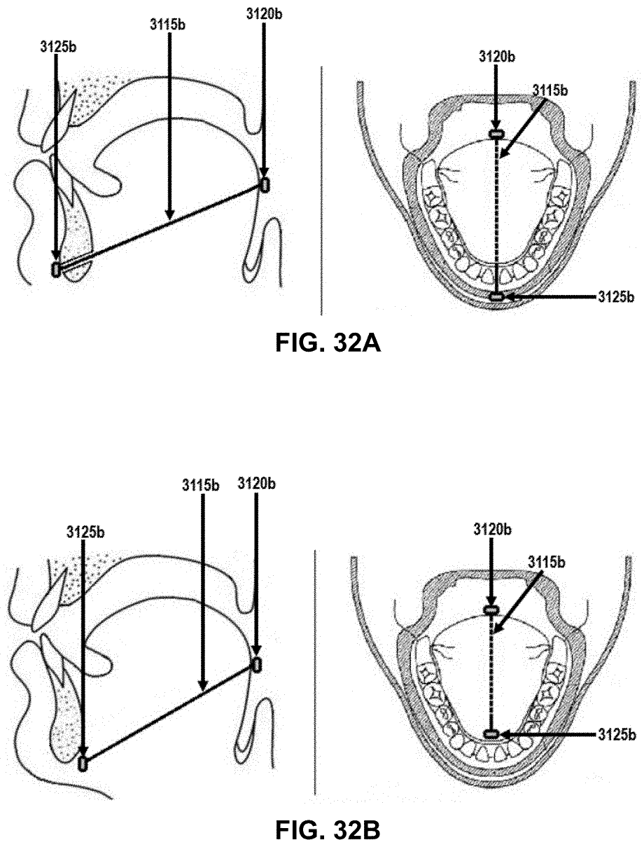

FIG. 32A illustrates an embodiment in which the multi-component device includes a retractor member 3120b, an anchor member 3125b, and an elongate member 3115b extending between the retractor member 3120b and the anchor member 3125b. In FIG. 32A, an anchor hole may be created. For example, an anchor hole may be drilled through the mandible 118 and the elongate member 3115b may be positioned to extend through the anchor hole. For example, the elongate member 3115b may extend from the anchor member 3125b provided at an anterior wall of the mandible 118 through the mandible 118 and the tissue of tongue 112, exit the tissue of the tongue 112, connect to the retractor member 3120b, and then extend from the first retractor member 3120b through the tissue of tongue 112 and the mandible 118 to the anchor member 3125b. The anchor member 3125b and a first end of the elongate member 3125b may be located at the entrance of the anchor hole and external to the mandible 118, and the second end of the elongate member 3125b may be located at the oropharynx 115. A first end of the elongate member 3115b may be connected to the anchor member 3125b, and a second end of the elongate member 3115b may be connected to the retractor member 3120b. For example, when the retractor member 3120b and/or the anchor member 3125b is comprised of stud head 600m, one end of the elongate member 3115b may be slipped through the hole of a flange F of stud head 600m, adjusted in length, and then forcibly pulled through the hole of an O-ring OR of stud head 600m that locks the flange F of the retractor member 3120b against the tongue 112 and/or the anchor member 3125b against the mandible 118, respectively. As another example, when the retractor member 3120b and/or the anchor member 3125b is comprised of stud head 600n, one end of the elongate member 3115b may be slipped through the first and second holes of a flange F of stud head 600n, adjusted in length, and knotted off to lock the flange F of the retractor member 3120b against the tongue 112 and/or the anchor member 3125b against the mandible 118, respectively. As shown in FIG. 32A, the elongate member 3115a may be inserted through the mandible 118 and the tongue 112 or tongue base 113 at a midline position of the mandible 118 and the tongue 112 or tongue base 113, respectively.

FIG. 32B illustrates an embodiment in which the multi-component device includes a retractor member 3120b, an anchor member 3125b, and an elongate member 3115b extending between the retractor member 3120b and the anchor member 3125b. In FIG. 32B, the elongate member 3115b may extend from the anchor member 3125b provided at a position below the mandible 118 through the tissue of tongue 112, exit the tissue of the tongue 112, connect to the retractor member 3120b, and then extend from the first retractor member 3120b through the tissue of tongue 112 to the anchor member 3125b. The anchor member 3125b and a first end of the elongate member 3125b may be located at a position below the mandible 118 in the facial tissue, and the second end of the elongate member 3125b may be located at the oropharynx 115. For example, the anchor member 3125b and the first end of the elongate member 3115b may be located at a midline region of the mandible 118. A first end of the elongate member 3115b may be connected to the anchor member 3125b, and a second end of the elongate member 3115b may be connected to the retractor member 3120b. Examples of the retractor member 3120b and the anchor member 3125b of FIG. 32B, and their respective connections with the elongate member 3115b, may be the same as those discussed above in connection with FIG. 32A.

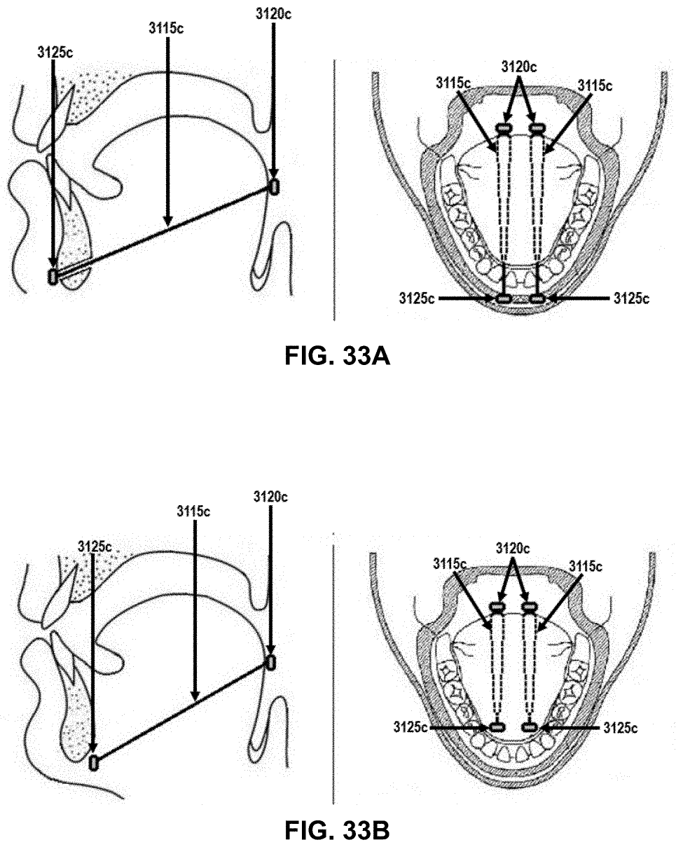

FIG. 33A illustrates an embodiment in which the multi-component device includes two anchor members 3125c, two elongate members 3115c, and two retractor members 3120c. In FIG. 33A, two anchor holes may be created. For example, two anchor holes may be drilled through the mandible 118 and each of the elongate members 3115c may be positioned to extend through a corresponding one of the anchor holes. For example, a first elongate member 3115c may extend from a first anchor member 3125a provided at an anterior wall of the mandible 118 through a first anchor hole in the mandible 118 and the tissue of tongue 112, exit the tissue of the tongue 112 at the base of the tongue 112, connect to a first retractor member 3120c, and then extend from the first retractor member 3120c through the tissue of tongue 112 and the first anchor hole of the mandible 118 to the first anchor member 3125c. The first anchor member 3125c and first and second ends of the first elongate member 3115c may be located at the entrance of the first anchor hole and external to the mandible 118. A second elongate member 3115c may extend from a second anchor member 3125a provided at an anterior wall of the mandible 118 through a second anchor hole in the mandible 118 and the tissue of tongue 112, exit the tissue of the tongue 112 at the base of the tongue 112, connect to a second retractor member 3120c, and then extend from the second retractor member 3120c through the tissue of tongue 112 and the second anchor hole of the mandible 118 to the second anchor member 3125c. The second anchor member 3125c and first and second ends of the second elongate member 3115c may be located at the entrance of the second anchor hole and external to the mandible 118. The first and second elongate members 3115c may be connected to the first and second retractor members 3120c, respectively, by loops. For example, when the retractor member 3120c is comprised of stud head 600h, the elongate member 3115c may extend through the hole of a flange F and loop around a button B of stud head 600h of the retractor member 3120c. As another example, when the retractor member 3120c is comprised of stud head 600i, the elongate member 3115c may be looped through the holes of the flange F of stud head 600i of the retractor member 3120c. The first and second ends of the elongate member 3115c may be pulled through the anchor member 3125c to adjust the tension before securement of the first and second ends of the elongate member 3115c to the anchor member 3125c. For example, when the anchor member 3125c is comprised of stud head 600m, the first and second ends of the elongate member 3115c may be slipped through the hole of a flange F of stud head 600m of the anchor member 3125c, adjusted in length, and then forcibly pulled through the hole of an O-ring OR of stud head 600m of the anchor member 3125c that locks the flange F against the mandible 118. As another example, when the anchor member 3125c is comprised of stud head 600n, the first and second ends of the elongate member 3125c may be slipped through the first and second holes of a flange F of stud head 600n of the anchor member 3125c, respectively, and knotted off to lock the flange F against the mandible 118. As shown in FIG. 33A, the elongate member 3115c may be inserted through the mandible 118 and tongue 112 or tongue base 113 at positions offset from a midline position of the mandible 118 and tongue 112 or tongue base 113, respectively.

FIG. 33B illustrates an embodiment in which the multi-component device includes two anchor members 3125c, two elongate members 3115c, and two retractor members 3120c. In FIG. 33B, a first elongate member 3115c may extend from a first anchor member 3125a through the tissue of tongue 112, exit the tissue of the tongue 112 at the base of the tongue 112, connect to a first retractor member 3120c, and then extend from the first retractor member 3120c through the tissue of tongue 112 to the first anchor member 3125c. The first anchor member 3125c and first and second ends of the first elongate member 3115c may be located at a position below the mandible 118 in the facial tissue. For example, the first anchor member 3125c and the first and second ends of the first elongate member 3115c may be located in a region subjacent to the mandible 118 at a location that is lateral to a midline region of the mandible 118. A second elongate member 3115c may extend from a second anchor member 3125a through the tissue of tongue 112, exit the tissue of the tongue 112 at the base of the tongue 112, connect to a second retractor member 3120c, and then extend from the second retractor member 3120c through the tissue of tongue 112 to the second anchor member 3125c. The second anchor member 3125c and first and second ends of the second elongate member 3115c may be located at a position below the mandible 118 in the facial tissue. For example, the second anchor member 3125c and the first and second ends of the second elongate member 3115c may be located in a region subjacent to the mandible 118 at a location that is contralateral to the midline region of the mandible 118. Examples of the retractor members 3120c and the anchor members 3125c of FIG. 33B, and their respective connections with the elongate members 3115c, may be the same as those discussed above in connection with FIG. 33A.

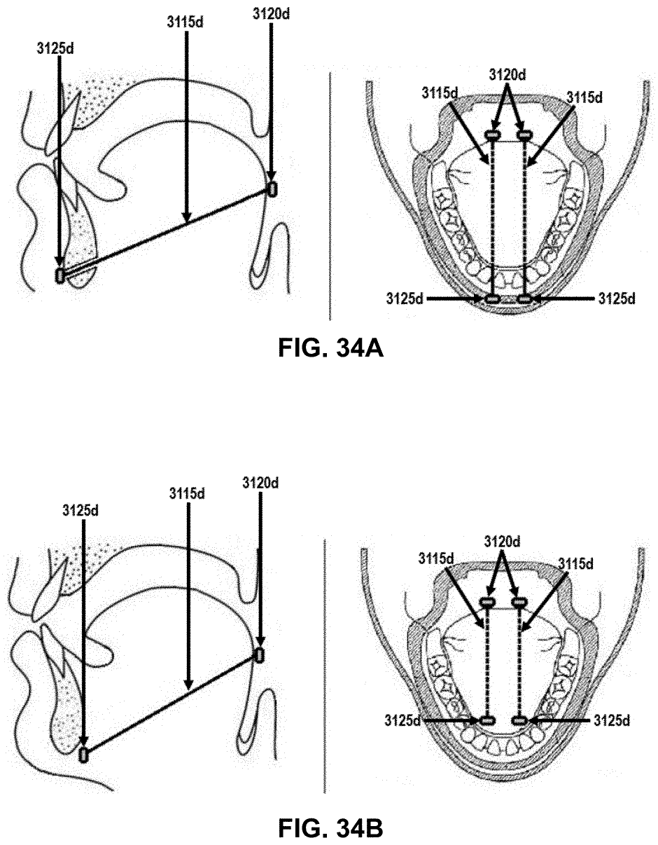

FIG. 34A illustrates an embodiment in which the multi-component device includes two anchor members 3125d, two elongate members 3115d, and two retractor members 3120d. In FIG. 34A, two anchor holes may be created. For example, two anchor holes may be drilled through the mandible 118 and each of the elongate members 3115d may be positioned to extend through a corresponding one of the anchor holes. For example, a first elongate member 3115d may extend from a first anchor member 3125d provided at an anterior wall of the mandible 118 through a first anchor hole in the mandible 118 and the tissue of tongue 112, exit the tissue of the tongue 112 at the base of the tongue 112, and connect to a first retractor member 3120d. The first anchor member 3125d and a first end of the first elongate member 3115d may be located at the entrance of the first anchor hole and external to the mandible 118, and the first retractor member 3120d and a second end of the first elongate member 3115d may be located in the oropharynx 115 and external to the tongue 112. A second elongate member 3115d may extend from a second anchor member 3125d provided at an anterior wall of the mandible 118 through a second anchor hole in the mandible 118 and the tissue of tongue 112, exit the tissue of the tongue 112 at the base of the tongue 112, and connect to a second retractor member 3120d. The second anchor member 3125d and a first end of the second elongate member 3115d may be located at the entrance of the second anchor hole and external to the mandible 118, and the second retractor member 3120d and a second end of the second elongate member 3115d may be located in the oropharynx 115 and external to the tongue 112. A first end of each of the first and second elongate members 3115d may be connected to the first and second anchor members 3125d, respectively, and a second end of each of the first and second elongate members 3115d may be connected to the first and second retractor members 3120d, respectively. For example, when the retractor members 3120d and/or the anchor members 3125d are comprised of stud head 600m, one end of the elongate member 3115d may be slipped through the hole of a flange F of stud head 600m, adjusted in length, and then forcibly pulled through the hole of an O-ring OR of stud head 600m that locks the flange F of the retractor member 3120d against the tongue 112 and/or the anchor member 3125d against the mandible 118, respectively. As another example, when the retractor members 3120d and/or the anchor members 3125d are comprised of stud head 600n, one end of the elongate member 3115d may be slipped through the first and second holes of a flange F of stud head 600n, adjusted in length, and knotted off to lock the flange F of the retractor member 3120d against the tongue 112 and/or the anchor member 3125d against the mandible 118, respectively. As shown in FIG. 34A, the elongate member 3115d may be inserted through the mandible 118 and the tongue 112 or tongue base 113 at a position offset from a midline position of the mandible 118 and the tongue 112 or tongue base 113, respectively.

FIG. 34B illustrates an embodiment in which the multi-component device includes two anchor members 3125d, two elongate members 3115d, and two retractor members 3120d. In FIG. 34B, a first elongate member 3115d may extend from a first anchor member 3125d through the tissue of tongue 112, exit the tissue of the tongue 112 at the base of the tongue 112, and connect to a first retractor member 3120d. The first anchor member 3125d and a first end of the first elongate member 3115d may be located at a position below the mandible 118 within the facial tissue, and the first retractor member 3120d and a second end of the first elongate member 3115d may be located in the oropharynx 115 and external to the tongue 112. For example, the first anchor member 3125d and a first end of the first elongate member 3115d may be located in a region subjacent to the mandible 118 at a location that is lateral to a midline region of the mandible 118, and the second end of the first elongate member 3115d may be located in a region of the pharynx that is lateral to a midline region of the tongue 112. A second elongate member 3115d may extend from a second anchor member 3125d through the tissue of tongue 112, exit the tissue of the tongue 112 at the base of the tongue 112, and connect to a second retractor member 3120d. The second anchor member 3125d and a first end of the second elongate member 3115d may be located at a position below the mandible 118 and in the facial tissue, and the second retractor member 3120d and a second end of the second elongate member 3115d may be located in the oropharynx 115 and external to the tongue 112. For example, the second anchor member 3125d and a first end of the second elongate member 3115d may be located in a region subjacent to the mandible 118 at a location that is contralateral to the midline region of the mandible 118, and the second end of the second elongate member 3115d may be located in a region of the pharynx that is contralateral to a midline region of the tongue 112. A first end of each of the first and second elongate members 3115d may be connected to the first and second anchor members 3125d, respectively, and a second end of each of the first and second elongate members 3115d may be connected to the first and second retractor members 3120d, respectively. Examples of the retractor member 3120d and the anchor member 3125d of FIG. 34B, and their respective connections with elongate members 3115d, may be the same as those discussed above in connection with FIG. 34A.

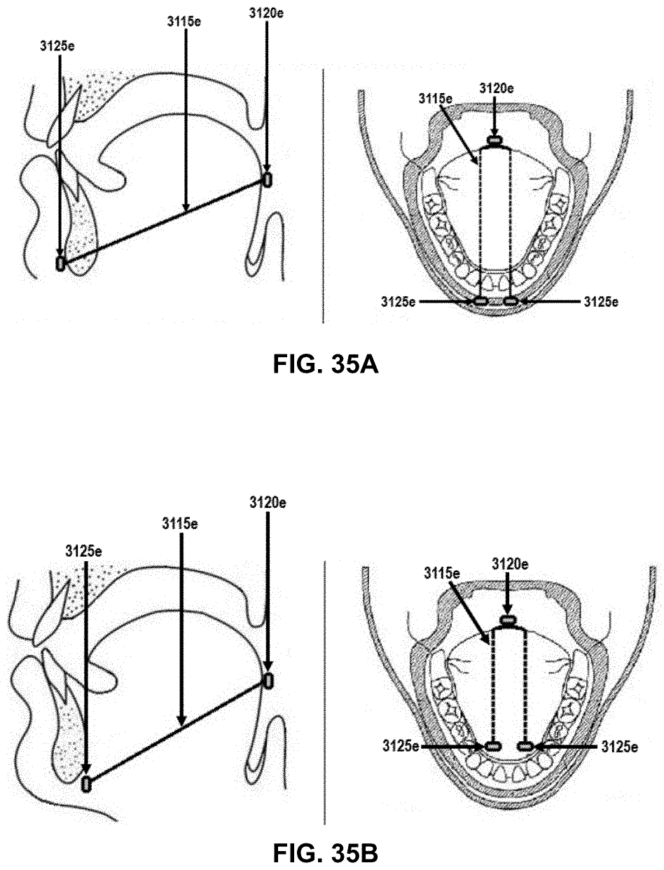

FIG. 35A illustrates an embodiment in which the multi-component device includes two anchor members 3125e, one elongate member 3115e, and one retractor member 3115e. In FIG. 35A, two anchor holes may be created. For example, two anchor holes may be drilled through the mandible 118 and the elongate member 3115e may be provided to extend through each of the two anchor holes. For example, the elongate member 3115e may extend from a first anchor member 3125e provided at an anterior wall of the mandible 118, through a first anchor hole and the tissue of tongue 112, exit the tissue of the tongue 112, connect to retractor member 3115e, and then extend back through the tissue of the tongue 112 and through the second anchor hole to the anterior wall of the mandible 118. The first and second ends of the elongate member 3115e may be pulled through the first and second anchor members 3125e, respectively, to adjust the tension of the elongate member 3115e before securement of the first and second ends of the elongate members 3115e to the first and second anchor members 3125e. The first anchor member 3125e and a first end of the elongate member 3115e may be located at the entrance of the first anchor hole and external to the mandible 118, the second anchor member 3125e and a second end of the elongate member 3115e may be located at the entrance of the second anchor hole and external to the mandible 118, and the first retractor member 3120e may be located in the oropharynx 115 and external to the tongue 112. A portion of the elongate member 3115e may comprise a loop that is connected to the retractor member 3120e. A first end of the elongate member 3115e may be connected to the first anchor member 3125e, and a second end of the elongate member 3115e may be connected to the second anchor member 3125e. For example, when the anchor members 3125e are comprised of stud head 600m, one end of the elongate member 3115e may be slipped through the hole of a flange F of stud head 600m, adjusted in length, and then forcibly pulled through the hole of an O-ring OR of stud head 600m that locks the flange F of the anchor member 3125e against the mandible 118. As another example, when the anchor members 3125e are comprised of stud head 600n, one end of the elongate member 3115e may be slipped through the first and second holes of a flange F of stud head 600n, adjusted in length, and knotted off to lock the flange F of the anchor member 3125e against the mandible 118. As shown in FIG. 35A, the elongate member 3115e may be inserted through the mandible 118 and the tongue 112 or tongue base 113 at a position offset from a midline position of the mandible 118 and the tongue 112 or tongue base 113 respectively.

FIG. 35B illustrates an embodiment in which the multi-component device includes two anchor members 3125e, one elongate member 3115e, and one retractor member 3115e. In FIG. 35B, the elongate member 3115e may extend from a first anchor member 3125e the tissue of tongue 112, exit the tissue of the tongue 112, connect to retractor member 3115e, and then extend back through the tissue of the tongue 112 and to the second anchor member 3125e. The first and second ends of the elongate member 3115e may be pulled through the first and second anchor members 3125e, respectively, to adjust the tension of the elongate member 3115e before securement of the first and second ends of the elongate members 3115e to the first and second anchor members 3125e. The first anchor member 3125e and a first end of the elongate member 3115e may be located at a position below the mandible 118 and within the facial tissue offset to a first side, the second anchor member 3125e and a second end of the elongate member 3115e may be located at a position below the mandible 118 and in the facial tissue offset to a second side, and the first retractor member 3120e may be located in the oropharynx 115 and external to the tongue 112. For example, the first anchor member 3125e and a first end of the elongate member 3115e may be located in a region subjacent to the mandible 118 at a location that is lateral to a midline region of the mandible 118, the second anchor member 3125e and a second end of the elongate member 3115e may be located in a region subjacent to the mandible 118 at a location that is lateral to a midline region of the mandible 118, and the first retractor member 3120e may be located in a region of the pharynx. A portion of the elongate member 3115e may comprise a loop that is connected to the retractor member 3120e. A first end of the elongate member 3115e may be connected to the first anchor member 3125e, and a second end of the elongate member 3115e may be connected to the second anchor member 3125e. Examples of the retractor member 3120a and the anchor members 3125a of FIG. 35B, and their respective connections with the elongate member 3115e, may be the same as those discussed above in connection with FIG. 35A.

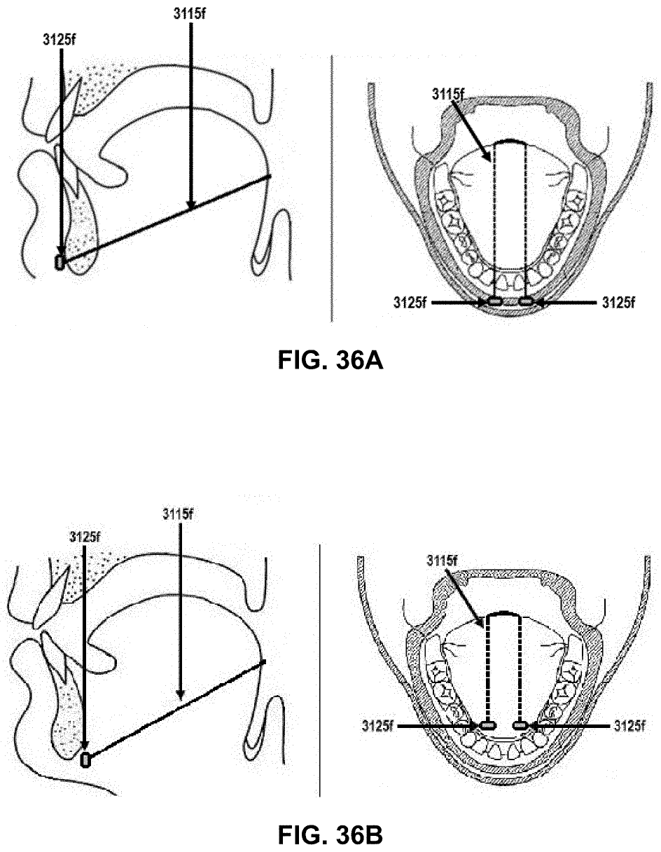

FIG. 36A illustrates an embodiment in which the multi-component device includes two anchor members 3125f and one elongate member 3115f. In FIG. 36A, two anchor holes may be created. For example, two anchor holes may be drilled through the mandible 118 and the elongate member 3115f may be provided to extend through each of the two anchor holes. For example, the elongate member 3115f may extend from a first anchor member 3125f provided at an anterior wall of the mandible 118, through a first anchor hole and the tissue of tongue 112, exit the tissue of the tongue 112, loop over the surface of the tongue 112, and then extend back through the tissue of the tongue 112 and through the second anchor hole to the anterior wall of the mandible 118. The first and second ends of the elongate member 3115f may be pulled through the first and second anchor members 3125f, respectively, to adjust the tension of the elongate member 3115f before securement of the first and second ends of the elongate members 3115f to the first and second anchor members 3125f. The first anchor member 3125f and a first end of the elongate member 3115f may be located at the entrance of the first anchor hole and external to the mandible 118, and the second anchor member 3125f and a second end of the elongate member 3115f may be located at the entrance of the second anchor hole and external to the mandible 118. A first end of the elongate member 3115f may be connected to the first anchor member 3125f, and a second end of the elongate member 3115f may be connected to the second anchor member 3125f. For example, when the anchor members 3125f are comprised of stud head 600m, one end of the elongate member 3115f may be slipped through the hole of a flange F of stud head 600m, adjusted in length, and then forcibly pulled through the hole of an O-ring OR of stud head 600m that locks the flange F of the anchor member 3125f against the mandible 118. As another example, when the anchor members 3125f are comprised of stud head 600n, one end of the elongate member 3115f may be slipped through the first and second holes of a flange F of stud head 600n, adjusted in length, and knotted off to lock the flange F of the anchor member 3125f against the mandible 118. As shown in FIG. 36A, the elongate member 3115f may be inserted through the mandible 118 and the tongue 112 or tongue base 113 at a position offset from a midline position of the mandible 118 and the tongue 112 or tongue base 113, respectively.

FIG. 36B illustrates an embodiment in which the multi-component device includes two anchor members 3125f and one elongate member 3115f. In FIG. 36B, the elongate member 3115f may extend from a first anchor member 3125f through the tissue of tongue 112, exit the tissue of the tongue 112, loop over the surface of the tongue 112, and then extend back through the tissue of the tongue 112 to the second anchor member 3125f. The first and second ends of the elongate member 3115f may be pulled through the first and second anchor members 3125f, respectively, to adjust the tension of the elongate member 3115f before securement of the first and second ends of the elongate members 3115f to the first and second anchor members 3125f. The first anchor member 3125f and a first end of the elongate member 3115f may be located at a position below the mandible 118 and in the facial tissue offset to a first side, and the second anchor member 3125f and a second end of the elongate member 3115f may be located at a position below the mandible 118 and in the facial tissue offset to a second side. For example, the first anchor member 3125f and a first end of the elongate member 3115f may be located in a region subjacent to the mandible 118 at a location that is lateral to a midline region of the mandible 118, and the second anchor member 3125f and a second end of the elongate member 3115f may be located in a region subjacent to the mandible 118 at a location that is lateral to a midline region of the mandible 118. A first end of the elongate member 3115f may be connected to the first anchor member 3125f, and a second end of the elongate member 3115f may be connected to the second anchor member 3125f. Examples of the anchor members 3125f of FIG. 36B, and their respective connections to the elongate member 3115f, may be the same as those discussed above in connection with FIG. 36A.

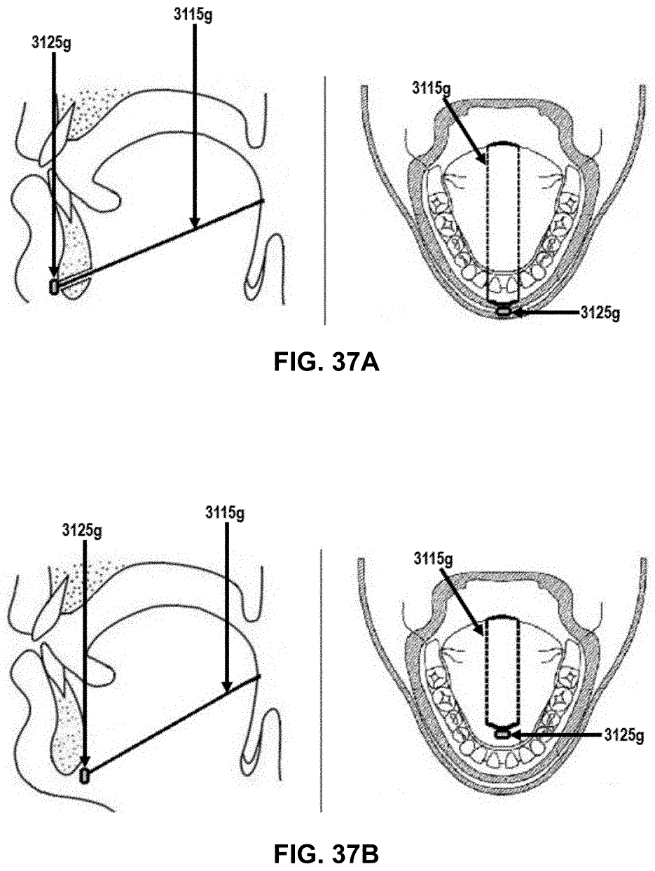

FIG. 37A illustrates an embodiment in which the multi-component device includes one anchor member 3125g and one elongate member 3115g. In FIG. 37A, two anchor holes may be created. For example, two anchor holes may be drilled through the mandible 118 and the elongate member 3115g may be provided to extend through each of the two anchor holes. For example, the elongate member 3115g may extend from the anchor member 3125g provided at an anterior wall of the mandible 118, through a first anchor hole and the tissue of tongue 112, exit the tissue of the tongue 112, loop over the surface of the tongue 112, and then extend back through the tissue of the tongue 112 and through the second anchor hole to the anchor member 3125g provided at the anterior wall of the mandible 118. The first and second ends of the elongate member 3115g may be pulled through the anchor members 3125g to adjust the tension of the elongate member 3115g before securement of the first and second ends of the elongate members 3115g to the anchor member 3125g. The anchor member 3125g and first and second ends of the elongate member 3115g may be located at a position between the entrances of the first and second anchor holes and external to the mandible 118. The first and second ends of the elongate member 3115g may be connected to the anchor member 3125g. For example, when the anchor member 3125g is comprised of stud head 600m, the first and second ends of the elongate member 3115g may be slipped through the hole of a flange F of stud head 600m, adjusted in length, and then forcibly pulled through the hole of an O-ring OR of stud head 600m that locks the flange F of the anchor member 3125g against the mandible 118. As another example, when the anchor member 3125g is comprised of stud head 600n, the first and second ends of the elongate member 3115g may be slipped through the first and second holes of a flange F of stud head 600n, adjusted in length, and knotted off to lock the flange F of the anchor member 3125g against the mandible 118. As shown in FIG. 37A, the elongate member 3115g may be inserted through the mandible 118 and the tongue 112 or tongue base 113 at positions offset from a midline position of the mandible 118 and the tongue 112 or tongue base 113, respectively.

FIG. 37B illustrates an embodiment in which the multi-component device includes one anchor member 3125g and one elongate member 3115g. In FIG. 37B, the elongate member 3115g may extend from the anchor member 3125g through the tissue of tongue 112, exit the tissue of the tongue 112, loop over the surface of the tongue 112, and then extend back through the tissue of the tongue 112 to the anchor member 3125g. The first and second ends of the elongate member 3115g may be pulled through the anchor members 3125g to adjust the tension of the elongate member 3115g before securement of the first and second ends of the elongate members 3115g to the anchor member 3125g. The anchor member 3125g and first and second ends of the elongate member 3115g may be located at a position below the mandible 118 and in the facial tissue. For example, the anchor member 3125g and first and second ends of the elongate member 3115g may be located at a midline region subjacent to the mandible 118. The first and second ends of the elongate member 3115g may be connected to the anchor member 3125g. Examples of the anchor member 3125g of FIG. 37B, and its connections to the elongate member 3115g, may be the same as those discussed above in connection with FIG. 37A.

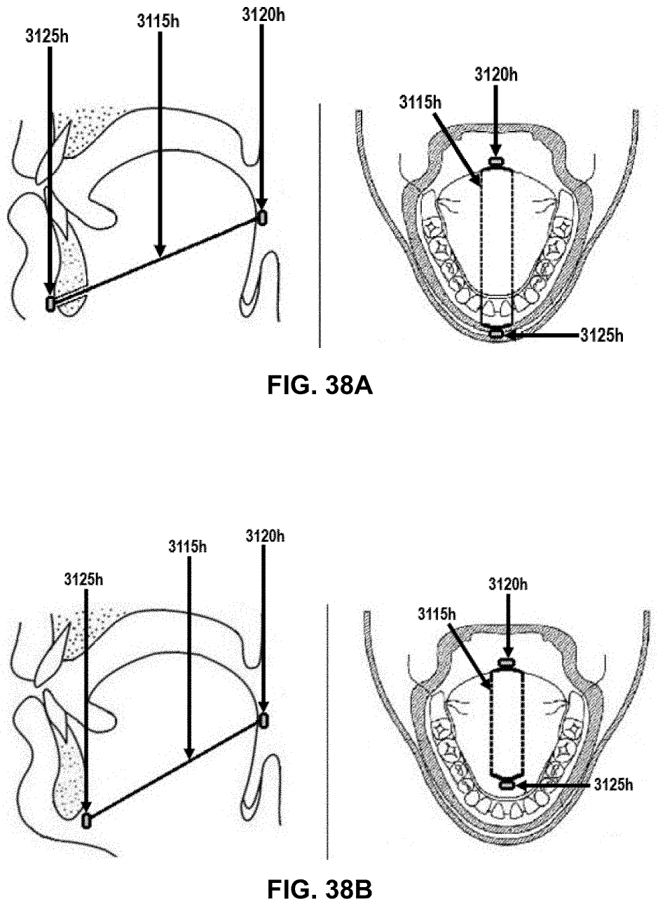

FIG. 38A illustrates an embodiment in which the multi-component device includes one anchor member 3125h, one elongate member 3115h, and one retractor member 3115h. In FIG. 38A, two anchor holes may be created. For example, two anchor holes may be drilled through the mandible 118 and the elongate member 3115h may be provided to extend through each of the two anchor holes. For example, the elongate member 3115h may extend from the anchor member 3125h provided at an anterior wall of the mandible 118, through a first anchor hole and the tissue of tongue 112, exit the tissue of the tongue 112, connect to retractor member 3115h, and then extend back through the tissue of the tongue 112 and through the second anchor hole to the anterior wall of the mandible 118. The first and second ends of the elongate member 3115h may be pulled through the anchor member 3125h to adjust the tension of the elongate member 3115h before securement of the first and second ends of the elongate members 3115h to the anchor member 3125h. The anchor member 3125h and the first and second ends of the elongate member 3115h may be located at a position between the first and second anchor holes and external to the mandible 118, and the first retractor member 3120h may be located in the oropharynx 115 and external to the tongue 112. A portion of the elongate member 3115h may comprise a loop that is connected to the retractor member 3120h. The first and second ends of the elongate member 3115h may be connected to the anchor member 3125h. For example, when the anchor member 3125h is comprised of stud head 600m, the first and second ends of the elongate member 3115h may be slipped through the hole of a flange F of stud head 600m, adjusted in length, and then forcibly pulled through the hole of an O-ring OR of stud head 600m that locks the flange F of the anchor member 3125h against the mandible 118. As another example, when the anchor member 3125h is comprised of stud head 600n, first and second ends of the elongate member 3115h may be slipped through the first and second holes of a flange F of stud head 600n, adjusted in length, and knotted off to lock the flange F of the anchor member 3125h against the mandible 118. As shown in FIG. 38A, the elongate member 3115h may be inserted through the mandible 118 and the tongue 112 or tongue base 113 at a position offset from a midline position of the mandible 118 and the tongue 112 or tongue base 113, respectively.

FIG. 38B illustrates an embodiment in which the multi-component device includes one anchor member 3125h, one elongate member 3115h, and one retractor member 3120h. In FIG. 38A, the elongate member 3115h may extend from the anchor member 3125h, through a first anchor hole and the tissue of tongue 112, exit the tissue of the tongue 112, connect to retractor member 3115h, and then extend back through the tissue of the tongue 112 and to the anchor member 3125h. The first and second ends of the elongate member 3115h may be pulled through the anchor member 3125h to adjust the tension of the elongate member 3115h before securement of the first and second ends of the elongate members 3115h to the anchor member 3125h. The anchor member 3125h and the first and second ends of the elongate member 3115h may be located at a position below the mandible 118 and in the facial tissue, and the first retractor member 3120h may be located in the oropharynx 115 and external to the tongue 112. For example, anchor member 3125h and the first and second ends of the elongate member 3115h may be located at a midline region subjacent to the mandible 118, and the first retractor member 3120h may be located at a midline region in the oropharynx 115. A portion of the elongate member 3115h may comprise a loop that is connected to the retractor member 3120h. The first and second ends of the elongate member 3115h may be connected to the anchor member 3125h. Examples of the anchor member 3125h of FIG. 38B, and its connections to the elongate member 3115h, may be the same as those discussed above in connection with FIG. 38A.

FIGS. 39A-39C, taken in conjunction with FIGS. 29A-29B and FIG. 30, illustrate the method of placement of components of a multi-component device used in glossomandibulopexy, consistent with certain exemplary embodiments. Specifically, each of FIGS. 39A-39C includes both a cross-sectional and endoscopic perspective of the tongue and surrounding structures to illustrate placement of components of a multi-component device used in glossomandibulopexy, consistent with certain exemplary embodiments. For example, FIGS. 39A-39C illustrate the embodiment of FIG. 32A, including one retractor member 3120b, one anchor member 3125b, and one elongate member 3115b extending between the retractor member 3120b and the anchor member 3125b.

Referring to (a) of FIG. 39A, FIGS. 29A-29B, and FIG. 30, the flexible fiberoptic endoscope may be positioned within the oropharynx 115 of a patient, and in (b) & (c), an anchor hole may be drilled in the mandible 118. Then, in (d), an insertion tool may be used to extend through the anchor hole and pierce the tongue 112, making a hole in the tongue 112. In some examples, the insertion tool may be a suture passer SP inserted through the tissue of tongue 112. In (d), the retractable stylet ST may be advanced beyond the tip of the canula CA to release a loop of suture SL beyond the tip of the stylet ST into the oropharynx 115 (see also (b) of FIG. 29B). In (e) of FIG. 39A, a hooking tool HT may be extended over the top of the tongue 112 to engage and retain the loop of suture SL that extends beyond the tip of the suture passer SP. In (f) of FIG. 39A, the canula CA and stylet ST may be withdrawn in unison and removed while the guidance tube GT and loop of suture SL remain in position within the tissue of tongue 112 (see also (c) and (d) of FIG. 29B). The tip of the guidance tube GT may be located at the surface of the tongue 112 in the oropharynx 115 and, in (f) of FIG. 39A, the hooking tool HT may draw the loop of suture over the top surface of the tongue 112 and external to the oral cavity 111. Once the loop is outside of the mouth, as shown in (g) of FIG. 39B, the loop of suture SL may be connected to an end of the shaft S of the oral stud, as shown in (h), and the oral stud may be drawn over the top surface of the tongue 112 to tightly contact the tip of the guidance tube GT in the oropharynx 115, as shown in (i) of FIG. 39A. The oral stud and guidance tube GT may be drawn forward in unison through the tissue of tongue 112 and outside of the mouth, as shown in (j) of FIG. 39B, such that the retractor member of the oral stud is pulled against the surface of the tongue, as shown in (k) of FIG. 39B. Then the loop of the suture SL and the guidance tube GT may be detached from the end of the shaft S, as shown in (l) of FIG. 39B, and an anchor member may be attached to the shaft, as shown in (m) of FIG. 39C. Finally, as shown in (n) of FIG. 39C, the shaft S may be adjusted in length to draw the retaining member and the anchor member against the external surface of the tongue 112. At each of steps (a) through (n) of FIGS. 39A-39C, the endoscopic helmet camera system of FIG. 30 may display images captured from the oropharynx 115 onto a monitor.

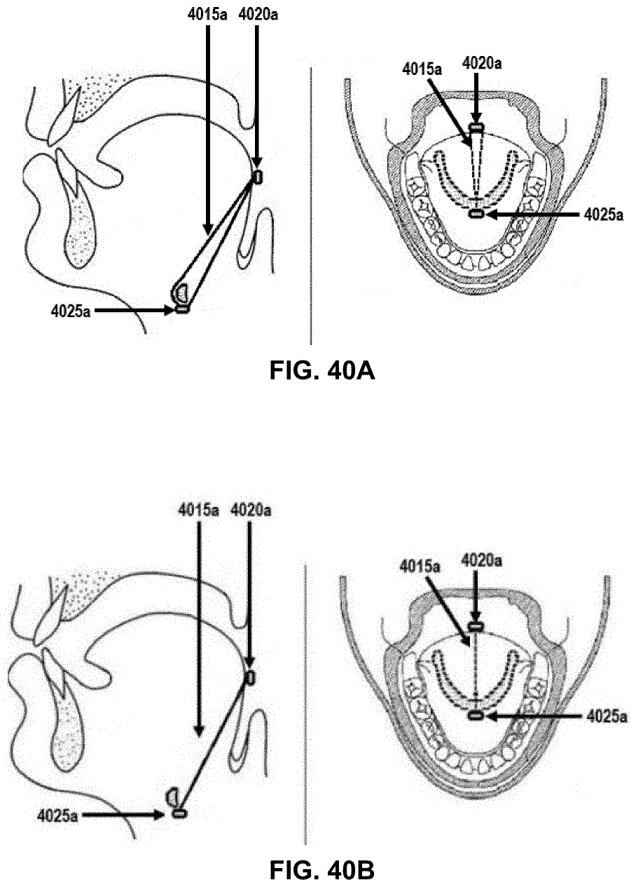

FIGS. 40A-40B, 41, and 42A-42B are front and side views of a human head to illustrate placement of components of a multi-component device used in suspension glossohyoidopexy, consistent with certain exemplary embodiments. In the embodiments illustrated by FIGS. 40A-40B, 41, and 42A-42B, the multi-component device may include at least one retractor member 4020, at least one anchor member 4025, and at least one elongate member 4015 extending between the at least one retractor member 4020 and the at least one anchor member 4025. The multi-component device illustrated by FIGS. 40A-40B, 41, and 42A-42B may be used to alter the position of the tongue 112 and, in particular, move the tongue 112 anteriorly away from the oropharynx 115. For example, the tongue 112 may be shifted slightly forward in the oral cavity 111.

The at least one elongate member 4015 may be inserted into and extend through a patient's oral tissue, such as, for example, a uvula 104, a tongue 112 or tongue base 113, or lateral pharyngeal walls 117. The at least one elongate member 4015 may be made of a biocompatible material, such as, for example, metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof. The at least one elongate member 4015 may include a shape memory material (SMM), such that the at least one elongate member 4015 is able to maintain and/or recover its original shape from a significant and seemingly plastic deformation when a particular stimulus is applied (e.g., superelasticity, visco-elasticity). For example, in each of the embodiments of FIGS. 40A-40B, 41, and 42A-42B, the at least one elongate member 4015 may be formed of a material having superelasticity, such that the force of the at least one elongate member 4015 returning to its original shape causes a gentle, continuous pressure to be applied to the at least one retractor member 4020 and the at least one anchor member 4025 to which the at least one elongate member 4015 is connected. Each of the at least one retractor members 4020 and the at least one anchor members 4025 may be, for example, the stud heads 600 of FIGS. 6H-6N. In example embodiments, one or more of the at least one retractor member 4020 and the at least one anchor members 4025 may be provided in the region of the pharynx, a region of the tongue, a region of the mandible bone, or a region of the hyoid bone, as discussed below.

FIG. 40A illustrates an embodiment in which the multi-component device includes one retractor member 4020a, one anchor member 4025a, and one elongate member 4015a extending between the retractor member 4020a and the anchor member 4025a. In FIG. 40A, the elongate member 4015a may extend from the anchor member 4025a provided at the hyoid bone through the tissue of tongue 112, exit the tissue of the tongue 112 at the base of the tongue 112, connect to the retractor member 4020a, and then extend from the retractor member 4020a through the tissue of tongue 112 to the anchor member 4025a. The anchor member 4025a and first and second ends of the elongate member 4015a may be located adjacent to the hyoid bone. For example, the anchor member 4025a may be subjacent to and in contact with a surface of the hyoid bone. The elongate member 4015a may be connected to the retractor member 4020a by a loop. For example, when the retractor member 4020a is comprised of stud head 600h, the elongate member 4015a may extend through the hole of a flange F and loop around a button B of stud head 600h of the retractor member 4020a. As another example, when the retractor member 4020a is comprised of stud head 600i, the elongate member 4015a may be looped through the holes of the flange F of stud head 600i of the retractor member 4020a. The first and second ends of the elongate member 4015a may be pulled through the anchor member 4025a to adjust the tension before securement of the first and second ends of the elongate member 4015a to the anchor member 4025a. For example, when the anchor member 4025a is comprised of stud head 600m, the first and second ends of the elongate member 4015a may be slipped through the hole of a flange F of stud head 600m of the anchor member 4025a, adjusted in length, and then forcibly pulled through the hole of an O-ring OR of stud head 600m of the anchor member 4025a that locks the flange F against the hyoid bone. As another example, when the anchor member 4025a is comprised of stud head 600n, the first and second ends of the elongate member 4025a may be slipped through the first and second holes of a flange F of stud head 600n of the anchor member 4025a, respectively, and knotted off to lock the flange F against the hyoid bone. As shown in FIG. 40A, the elongate member 4015a may be inserted through the tongue 112 or tongue base 113 at a midline position of the tongue 112 or tongue base 113, respectively.

FIG. 40B illustrates an embodiment in which the multi-component device includes one retractor member 4020a, one anchor member 4025a, and one elongate member 4015a extending between the retractor member 4020a and the anchor member 4025a. In FIG. 40B, the elongate member 4015a may extend from the anchor member 4025a provided at the hyoid bone through the tissue of tongue 112, exit the tissue of the tongue 112 at the base of the tongue 112, and connect to the retractor member 4020a. The anchor member 4025a and the elongate member 4015a may be located adjacent to the hyoid bone. For example, the anchor member 4025a may be subjacent to and in contact with a surface of the hyoid bone. The elongate member 4015a may be connected to the retractor member 4020a. For example, when the anchor member 4025a is comprised of stud head 600m, the end of the elongate member 4015a may be slipped through the hole of a flange F of stud head 600m of the anchor member 4025a, adjusted in length, and then forcibly pulled through the hole of an O-ring OR of stud head 600m of the anchor member 4025a that locks the flange F against the hyoid bone. As another example, when the anchor member 4025a is comprised of stud head 600n, the first and second ends of the elongate member 4025a may be slipped through the first and second holes of a flange F of stud head 600n of the anchor member 4025a, respectively, and knotted off to lock the flange F against the hyoid bone. As shown in FIG. 40B, the elongate member 4015a may be inserted through the tongue 112 or tongue base 113 at a midline position of the tongue 112 or tongue base 113, respectively.

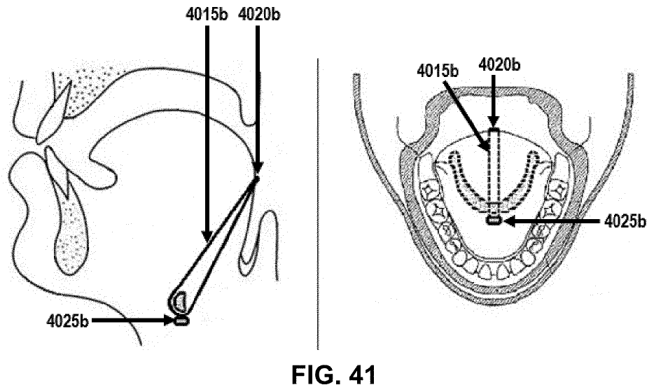

FIG. 41 illustrates an embodiment in which the multi-component device includes one anchor member 4025b and one elongate member 4015b. In FIG. 41, the elongate member 4015b may extend from the anchor member 4025b provided at the hyoid bone through the tissue of tongue 112, exit the tissue of the tongue 112 at the base of the tongue 112, extend across an external surface of the tongue 112, and then extend through the tissue of tongue 112 to the anchor member 4025b. The anchor member 4025b and first and second ends of the elongate member 4015b may be located adjacent to the hyoid bone. For example, the anchor member 4025b may be subjacent to and in contact with a surface of the hyoid bone. The first and second ends of the elongate member 4015b may be pulled through the anchor member 4025b to adjust the tension before securement of the first and second ends of the elongate member 4015b to the anchor member 4025b. For example, when the anchor member 4025b is comprised of stud head 600m, the first and second ends of the elongate member 4015b may be slipped through the hole of a flange F of stud head 600m of the anchor member 4025b, adjusted in length, and then forcibly pulled through the hole of an O-ring OR of stud head 600m of the anchor member 4025b that locks the flange F against the hyoid bone. As another example, when the anchor member 4025b is comprised of stud head 600n, the first and second ends of the elongate member 4025b may be slipped through the first and second holes of a flange F of stud head 600n of the anchor member 4025b, respectively, and knotted off to lock the flange F against the hyoid bone. As shown in FIG. 41, the elongate member 4015b may be inserted through the tongue 112 or tongue base 113 at a midline position of the tongue 112 or tongue base 113, respectively.

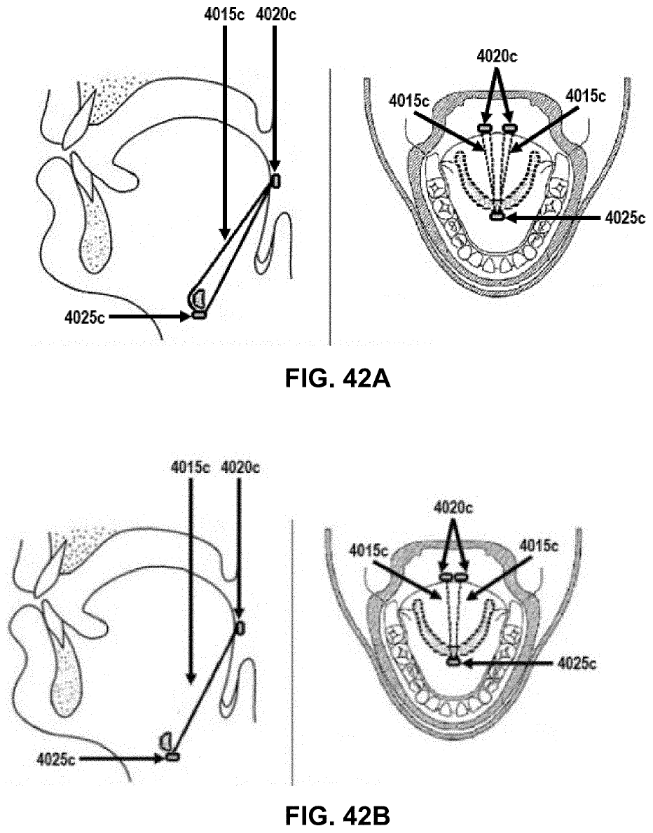

FIG. 42A illustrates an embodiment in which the multi-component device includes one anchor member 4025c, two retractor members 4020c, and two elongate members 4015c extending between the anchor member 4025c and the two retractor members 4020c. In FIG. 42A, a first elongate member 4015c may extend from the anchor member 4025c provided at the hyoid bone through the tissue of tongue 112, exit the tissue of the tongue 112 at the base of the tongue 112, connect to a first retractor member 4020c, and then extend through the tissue of tongue 112 to the anchor member 4025c. A second elongate member 4015c may extend from the anchor member 4025c provided at the hyoid bone through the tissue of tongue 112, exit the tissue of the tongue 112 at the base of the tongue 112, connect to a second retractor member 4020c, and then extend through the tissue of tongue 112 to the anchor member 4025c. The anchor member 4025c, first and second ends of the first elongate member 4015c, and first and second ends of the second elongate member 4015c may be located adjacent to the hyoid bone. For example, the anchor member 4025c may be subjacent to and in contact with a surface of the hyoid bone. The first and second ends of the elongate members 4015c may be pulled through the anchor member 4025c to adjust the tension before securement of the first and second ends of the elongate members 4015c to the anchor member 4025c. For example, when the anchor member 4025c is comprised of stud head 600m, the first and second ends of the first and second elongate members 4015c may be slipped through the hole of a flange F of stud head 600m of the anchor member 4025c, adjusted in length, and then forcibly pulled through the hole of an O-ring OR of stud head 600m of the anchor member 4025c that locks the flange F against the hyoid bone. As another example, when the anchor member 4025c is comprised of stud head 600n, the first and second ends of the first and second elongate members 4025c may be slipped through the first and second holes of a flange F of stud head 600n of the anchor member 4025c, respectively, and knotted off to lock the flange F against the hyoid bone. As shown in FIG. 42A, the elongate member 4015c may be inserted through the tongue 112 or tongue base 113 at a midline position of the tongue 112 or tongue base 113, respectively.

FIG. 42B illustrates an embodiment in which the multi-component device includes one anchor member 4025c, two retractor members 4020c, and two elongate members 4015c extending between the anchor member 4025c and the two retractor members 4020c. In FIG. 42B, a first elongate member 4015c may extend from the anchor member 4025c provided at the hyoid bone through the tissue of tongue 112, exit the tissue of the tongue 112 at the base of the tongue 112, and connect to a first retractor member 4020c. A second elongate member 4015c may extend from the anchor member 4025c provided at the hyoid bone through the tissue of tongue 112, exit the tissue of the tongue 112 at the base of the tongue 112, and connect to a second retractor member 4020c. The anchor member 4025c, first elongate member 4015c, and second elongate member 4015c may be located adjacent to the hyoid bone. For example, the anchor member 4025c may be subjacent to and in contact with a surface of the hyoid bone. The first ends of the first and second elongate members 4015c may be pulled through the anchor member 4025c to adjust the tension before securement of the elongate members 4015c to the anchor member 4025c. For example, when the anchor member 4025c is comprised of stud head 600m, the first ends of the first and second elongate members may be slipped through the hole of a flange F of stud head 600m of the anchor member 4025c, adjusted in length, and then forcibly pulled through the hole of an O-ring OR of stud head 600m of the anchor member 4025c that locks the flange F against the hyoid bone. As another example, when the anchor member 4025c is comprised of stud head 600n, the first ends of the first and second elongate members 4025c may be slipped through the first and second holes of a flange F of stud head 600n of the anchor member 4025c, respectively, and knotted off to lock the flange F against the hyoid bone. As shown in FIG. 42B, the elongate member 4015c may be inserted through the tongue 112 or tongue base 113 at a midline position of the tongue 112 or tongue base 113, respectively.

FIGS. 43A-43C, taken in conjunction with FIGS. 29A-29B and FIG. 30, illustrate the method of placement of components of a multi-component device used in glossohyoidopexy, consistent with certain exemplary embodiments. Specifically, each of FIGS. 43A-43C includes both a cross-sectional and endoscopic perspective of the tongue and surrounding structures to illustrate placement of components of a multi-component device used in glossohyoidopexy, consistent with certain exemplary embodiments. For example, FIGS. 43A-43C illustrate the embodiment of FIG. 40A, including one retractor member 4020a, one anchor member 4025a, and one elongate member 4015a extending between the retractor member 4020a and the anchor member 4025a.

Referring to (a) of FIG. 43A, FIGS. 29A-29B, and FIG. 40A, the flexible fiberoptic endoscope may be positioned within the oropharynx 115 of a patient, and in (b) an insertion tool may be used to pierce the skin overlying a hyoid bone and extending through the tongue 112, making a hole in the tongue 112. In some examples, the insertion tool may be a suture passer SP inserted through the tissue of tongue 112. The retractable stylet ST may be advanced beyond the tip of the canula CA to release a loop of suture SL beyond the tip of the stylet ST into the oropharynx 115 (see also (b) of FIG. 29B). In (c) of FIG. 43A, a hooking tool HT may be extended over the top of the tongue 112 to engage and retain the loop of suture SL that extends beyond the tip of the suture passer SP. In (d) of FIG. 43A, the canula CA and stylet ST may be withdrawn in unison and removed while the guidance tube GT and loop of suture SL remain in position within the tissue of tongue 112 (see also (c) and (d) of FIG. 29B). The tip of the guidance tube GT may be located at the surface of the tongue 112 in the oropharynx 115 and, in (e) of FIG. 43A, the hooking tool HT may draw the loop of suture over the top surface of the tongue 112 and external to the oral cavity 111. Once the loop is outside of the mouth, as shown in (f) of FIG. 43B, the loop of suture SL may be connected to an end of the shaft S of the oral stud, and the oral stud may be drawn over the top surface of the tongue 112 to tightly contact the tip of the guidance tube GT in the oropharynx 115, as shown in (g) of FIG. 43B. The oral stud and guidance tube GT may be drawn forward in unison through the tissue of tongue 112 and outside of the mouth, as shown in (h) of FIG. 43B, such that the retractor member of the oral stud is pulled against the surface of the tongue, as shown in (i) of FIG. 43B. Then the loop of the suture SL and the guidance tube GT may be detached from the end of the shaft S, as shown in (j) of FIG. 43B, and the suture SL may be looped around the hyoid bone, as shown in (k) of FIG. 43C. An anchor member may be attached to the shaft, as shown in (l) of FIG. 43C. Finally, as shown in (m) of FIG. 43C, the shaft S may be adjusted in length to draw the retaining member and the anchor member against the external surface of the tongue 112.

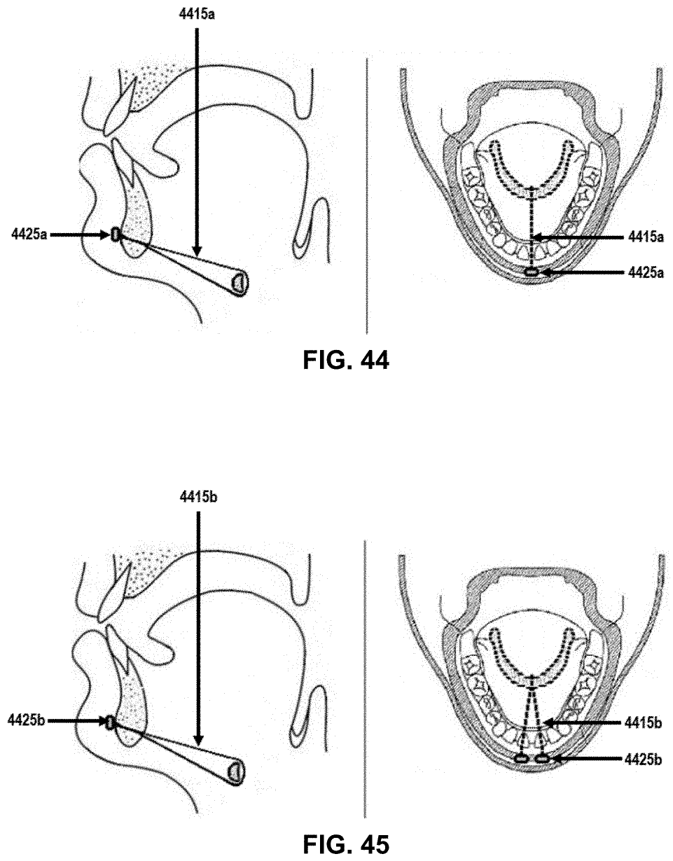

FIGS. 44-45 are front and side views of a human head to illustrate placement of components of a multi-component device used in suspension hyoidomandibulopexy, consistent with certain exemplary embodiments. In the embodiments illustrated by FIGS. 44-45, the multi-component device may include at least one anchor member 4425 and at least one elongate member 4415. The multi-component device illustrated by FIGS. 44-45 may be used to alter the position of the hyoid bone and, in particular, move the hyoid bone anteriorly away from the oropharynx 115. For example, the hyoid bone may be shifted slightly forward.

The at least one elongate member 4415 may be inserted into and extend through a patient's oral tissue, such as, for example, a mandible 118. The at least one elongate member 4415 may be made of a biocompatible material, such as, for example, metal (e.g., stainless steel, cobalt alloys, titanium alloys, etc.), a ceramic (e.g., aluminum oxide, zirconia, calcium phosphates, etc.), synthetic polymers (e.g., nylon, silicones, poly (ethylene), poly (vinyl chloride), polyurethanes, polylactides, etc.), natural polymers (e.g., collagen, gelatin, elastin, silk, polysaccharide, etc.), or any combination thereof. The at least one elongate member 4415 may include a shape memory material (SMM), such that the at least one elongate member 4415 is able to maintain and/or recover its original shape from a significant and seemingly plastic deformation when a particular stimulus is applied (e.g., superelasticity, visco-elasticity). For example, in each of the embodiments of FIGS. 44-45, the at least one elongate member 4415 may be formed of a material having superelasticity, such that the force of the at least one elongate member 4415 returning to its original shape causes a gentle, continuous pressure to be applied to the at least one retractor member 4420 and the at least one anchor member 3125 to which the at least one elongate member 4415 is connected. Each of the at least one retractor members 4420 and the at least one anchor members 4425 may be, for example, the stud heads 600 of FIGS. 6H-6N. In example embodiments, one or more of the at least one retractor member 4420 and the at least one anchor members 4425 may be provided in the region of the pharynx, a region of the tongue, a region of the mandible bone, or a region of the hyoid bone, as discussed below.

FIG. 44 illustrates an embodiment in which the multi-component device includes one anchor member 4425a and one elongate member 4415a. In FIG. 44, an anchor hole may be created. For example, an anchor hole may be drilled through the mandible 118 and the elongate member 4415a may be positioned to extend through the anchor hole. For example, the elongate member 4415a may extend from the anchor member 4425a provided at an anterior wall of the mandible 118 through the mandible 118, extend around the hyoid bone, and return to the anchor member 4425a through the mandible 118. The anchor member 4425a and first and second ends of the elongate member 4415a may be located at the entrance of the anchor hole and external to the mandible 118. The first and second ends of the elongate member 4415a may be pulled through the anchor member 4425a to adjust the tension before securement of the first and second ends of the elongate member 4415a to the anchor member 4425a. For example, when the anchor member 4425a is comprised of stud head 600m, the first and second ends of the elongate member 4415a may be slipped through the hole of a flange F of stud head 600m of the anchor member 4425a, adjusted in length, and then forcibly pulled through the hole of an O-ring OR of stud head 600m of the anchor member 4425a that locks the flange F against the mandible 118. As another example, when the anchor member 4425a is comprised of stud head 600n, the first and second ends of the elongate member 4425a may be slipped through the first and second holes of a flange F of stud head 600n of the anchor member 4425a, respectively, and knotted off to lock the flange F against the mandible 118. As shown in FIG. 44, the elongate member 4415a may be inserted through the mandible at a midline position of the mandible 118.

FIG. 45 illustrates an embodiment in which the multi-component device includes two anchor members 4425b and at least one elongate member 4415b. In FIG. 45, first and second anchor holes may be created. For example, first and second anchor holes may be drilled through the mandible 118, and first and second elongate members 4415b may be positioned to extend through the first and second anchor holes, respectively. For example, the first elongate member 4415b may extend from the first anchor member 4425b provided at an anterior wall of the mandible 118 through the mandible 118, extend around the hyoid bone, and return to the first anchor member 4425a through the first anchor hole of the mandible 118. The second elongate member 4415b may extend from the second anchor member 4425b provided at an anterior wall of the mandible 118 through the mandible 118 and around the hyoid bone, and return to the second anchor member 4425b through the second anchor hole of the mandible 118. The first anchor member 4425b and first and second ends of the first elongate member 4415b may be located at the entrance of the first anchor hole and external to the mandible 118, and the second anchor member 4425b and first and second ends of the second elongate member 4415b may be located at the entrance of the second anchor hole and external to the mandible 118. The first and second ends of the first and second elongate members 4415b may be pulled through the first and second anchor members 4425b, respectively, to adjust the tension before securement of the first and second ends of the elongate members 4415a to the first and second anchor members 4425b. For example, when the anchor members 4425b are comprised of stud head 600m, the first and second ends of the elongate member 4415b may be slipped through the hole of a flange F of stud head 600m of the anchor member 4425b, adjusted in length, and then forcibly pulled through the hole of an O-ring OR of stud head 600m of the anchor member 4425b that locks the flange F against the mandible 118. As another example, when the anchor member 4425b is comprised of stud head 600n, the first and second ends of the elongate member 4425b may be slipped through the first and second holes of a flange F of stud head 600n of the anchor member 4425b, respectively, and knotted off to lock the flange F against the mandible 118. As shown in FIG. 45, the elongate member 4415b may be inserted through the mandible at a midline position of the mandible 118.

The disclosed embodiments may minimize the amount of implanted material thus decreasing the risk for interference with the functional integrity of the structure, as well as, minimizing the chance for foreign body complications such as scarring and “foreign body” inflammatory reactions/extrusion.

The present disclosure is not to be limited in terms of the particular embodiments described in this application, which are intended as illustrations of various aspects. Many modifications and variations can be made without departing from its spirit and scope, as will be apparent. Functionally equivalent methods and apparatuses within the scope of the disclosure, in addition to those enumerated herein, will be apparent from the foregoing descriptions. Such modifications and variations are intended to fall within the scope of the appended claims. The present disclosure is to be limited only by the terms of the appended claims, along with the full scope of equivalents to which such claims are entitled. It is to be understood that this disclosure is not limited to particular methods, reagents, compounds compositions or biological systems, which can, of course, vary. It is also to be understood that the terminology used herein is for the purpose of describing particular embodiments only, and is not intended to be limiting.

The foregoing description, along with its associated embodiments, has been presented for purposes of illustration only. It is not exhaustive and does not limit the invention to the precise form disclosed. Those skilled in the art will appreciate from the foregoing description that modifications and variations are possible in light of the above teachings or may be acquired from practicing the disclosed embodiments. For example, the steps described need not be performed in the same sequence discussed or with the same degree of separation. Likewise various steps may be omitted, repeated, or combined, as necessary, to achieve the same or similar objectives. Accordingly, the invention is not limited to the above-described embodiments, but instead is defined by the appended claims in light of their full scope of equivalents.

1.-23. (canceled)

24. A method for treating a condition of an airway of a patient, comprising:
creating a first anchor hole at a first predetermined location within or subjacent to a mandible bone of the patient;
positioning a first elastic elongate member through the first anchor hole, the first elongate member having first and second ends at an entrance of the first anchor hole and a loop in a region of a pharynx of the patient;
connecting a first retractor member at or near an end of the loop of the first elastic elongate member in a region of a tongue of the patient; and
connecting a first anchor member at or near the first and second ends of the first elastic elongate member at the entrance of the first anchor hole,
wherein at least one of the first elastic elongate member, the first retractor member, and the first anchor member interact to distribute a force on the tongue and the force prevents obstruction of an airway of the patient.

25. The method of claim 24, wherein the first predetermined location is at a midline region of the mandible bone or at a midline region subjacent to the mandible bone.

26. The method of claim 24, wherein the first elastic elongate member, the first retractor member, and the first anchor member are each formed of a bio-compatible material.

27. The method of claim 24,
wherein the first elastic elongate member is positioned within soft tissue of the tongue, and
wherein the first retractor member is provided external to the soft tissue of the tongue.

28. The method of claim 24, further comprising:
creating a second anchor hole at a second predetermined location within or subjacent to the mandible bone;
positioning a second elastic elongate member through the second anchor hole, the second elongate member having first and second ends at an entrance of the second anchor hole and a loop in the region of the pharynx;
connecting a second retractor member at or near an end of the loop of the second elastic elongate member in the region of the tongue; and
connecting a second anchor member at or near the first and second ends of the second elastic elongate member at the entrance of the second anchor hole.

29. The method of claim 28,
wherein the first predetermined location is lateral to a midline region of the mandible bone or lateral to a midline region subjacent to the mandible bone, and
wherein the second predetermined location is contralateral to the midline region of the mandible bone or contralateral to the midline region subjacent to the mandible bone.

30. The method of claim 28,
wherein the first and second elastic elongate members are positioned within soft tissue of the tongue, and
wherein the first and second retractor members are provided external to the soft tissue of the tongue.

31. A method for treating a condition of an airway of a patient, comprising:
creating a first anchor hole at a first predetermined location within or subjacent to a mandible bone of the patient;
positioning a first elastic elongate member through the first anchor hole, the first elongate member having a first end at an entrance of the first anchor hole and a second end in a region of a pharynx of the patient;
connecting a first anchor member at or near the first end of the first elastic elongate member at the entrance of the first anchor hole; and
connecting a first retractor member at or near the second end of the first elastic elongate member in a region of a tongue of the patient,
wherein at least one of the first elastic elongate member, the first retractor member, and the first anchor member interact to distribute a force on the tongue and the force prevents obstruction of an airway of the patient.

32. The method of claim 31, wherein the first predetermined location is at a midline region of the mandible bone or at a midline region subjacent to the mandible bone.

33. The method of claim 31, wherein the first elastic elongate member, the first retractor member, and the first anchor member are each formed of a bio-compatible material.

34. The method of claim 31,
wherein the first elastic elongate member is positioned within soft tissue of the tongue, and
wherein the first retractor member is provided external to the soft tissue of the tongue.

35. The method of claim 31, further comprising:
creating a second anchor hole at a second predetermined location within or subjacent to the mandible bone;
positioning a second elastic elongate member through the second anchor hole, the second elongate member having a first end at an entrance of the second anchor hole and a second end in the region of the pharynx;
connecting a second anchor member at or near the first end of the second elastic elongate member at the entrance of the second anchor hole; and
connecting a second retractor member at or near the second end of the second elastic elongate member in the region of the tongue.

36. The method of claim 35,
wherein the first predetermined location is lateral to a midline region of the mandible bone or lateral to a midline region subjacent to the mandible bone, and
wherein the second predetermined location is contralateral to the midline region of the mandible bone or contralateral to the midline region subjacent to the mandible bone.

37. The method of claim 35,
wherein the first and second elastic elongate members are positioned within soft tissue of the tongue, and
wherein the first and second retractor members are provided external to the soft tissue of the tongue.

38. A method for treating a condition of an airway of a patient, comprising:
creating a first anchor hole at a first predetermined location within or subjacent to a mandible bone of the patient;
creating a second anchor hole at a second predetermined location within or subjacent to the mandible bone of the patient;
positioning an elastic elongate member through the first and second anchor holes, the first elongate member having a first end at an entrance of the first anchor hole, a second end at an entrance of the second anchor hole, and a loop in a region of a pharynx of the patient; and
connecting a first anchor member at or near the first end the elastic elongate member,
wherein at least one of the elastic elongate member and the first anchor member interact to distribute a force on a tongue of the patient and the force prevents obstruction of an airway of the patient.

39. The method of claim 38, further comprising:
connecting a second anchor member at or near the second end of the elastic elongate member.

40. The method of claim 39, further comprising:
connecting a retractor member at or near the loop of the elastic elongate member in a region of the tongue.

41. The method of claim 39, wherein the loop extends across a surface of the tongue.

42. The method of claim 38,
wherein the first predetermined location is lateral to a midline region of the mandible bone or lateral to a midline region subjacent to the mandible bone, and
wherein the second predetermined location is contralateral to the midline region of the mandible bone or contralateral to the midline region subjacent to the mandible bone.

43. The method of claim 38,
wherein the elastic elongate member is positioned within soft tissue of the tongue, and
wherein the loop is provided external to the soft tissue of the tongue.

44. A method for treating a condition of an airway of a patient, comprising:
positioning a first elastic elongate member in a soft tissue of a tongue of the patient, the first elastic elongate member having first and second ends in a midline region of a hyoid bone of the patient and a loop in a region of a pharynx of the patient; and
connecting an anchor member at or near the first and second ends of the first elastic elongate member in the midline region of the hyoid bone,
wherein at least one of the first elastic elongate member and the anchor member interact to distribute a force on the tongue of the patient and the force prevents obstruction of the airway of the patient.

45. The method of claim 44, further comprising:
connecting a first retractor member at or near the loop of the first elastic elongate member in a region of the tongue of the patient.

46. The method of claim 45, further comprising:
positioning a second elastic elongate member in the soft tissue of the tongue, the second elastic elongate member having first and second ends in the midline region of the hyoid bone and a loop in region of the pharynx; and
connecting the anchor member at or near the first and second ends of the second elastic elongate member in the midline region of the hyoid bone.

47. The method of claim 44, wherein the loop extends across a surface of the tongue.

48. A method for treating a condition of an airway of a patient, comprising:
positioning a first elastic elongate member in a soft tissue of a tongue of the patient, the first elastic elongate member having a first end in a midline region of a hyoid bone of the patient and a second end in a region of a pharynx of the patient;
connecting an anchor member at or near the first end of the first elastic elongate member in the midline region of the hyoid bone; and
connecting a first retractor member at or near the second end of the first elastic elongate member in a region of a tongue of the patient,
wherein at least one of the first elastic elongate member and the anchor member interact to distribute a force on the tongue of the patient and the force prevents obstruction of the airway of the patient.

49. The method of claim 48, further comprising:
positioning a second elastic elongate member in the soft tissue of the tongue, the second elastic elongate member having a first end in the midline region of the hyoid bone and a second end in the region of the pharynx; and
connecting the anchor member at or near the first and second ends of the second elastic elongate member in the midline region of the hyoid bone.

50. A method for treating a condition of an airway of a patient, comprising:
creating a first anchor hole at a first predetermined location of a mandible bone of the patient;
positioning a first elastic elongate member through the first anchor hole, the first elongate member having first and second ends at an entrance of the first anchor hole and a loop in a midline region of a hyoid bone of the patient; and
connecting a first anchor member at or near the first and second ends of the first elastic elongate member at the entrance of the first anchor hole,
wherein at least one of the first elastic elongate member and the first anchor member interact to distribute a force on the hyoid bone and the force prevents obstruction of an airway of the patient.

51. The method of claim 50, wherein the first predetermined location is at a midline region of the mandible bone.

52. The method of claim 50, further comprising:
creating a second anchor hole at a second predetermined location of the mandible bone;
positioning a second elastic elongate member through the second anchor hole, the second elongate member having first and second ends at an entrance of the second anchor hole and a loop at the midline region of the hyoid bone; and
connecting a second anchor member at or near the first and second ends of the second elastic elongate member at the entrance of the second anchor hole.

53. The method of claim 52,
wherein the first predetermined location is lateral to a midline region of the mandible bone, and
wherein the second predetermined location is contralateral to the midline region of the mandible bone.
